(19) United States
(12) Patent Application Publication
(10) Pub. No.: US 2025/0129137 A1
(43) Pub. Date: Apr. 24, 2025

(54) CD71 BINDING FIBRONECTIN TYPE III DOMAINS

(71) Applicant: Aro Biotherapeutics, Philadelphia, PA (US)

(72) Inventors: Russell C. Addis, Philadelphia, PA (US); Zhanna Druzina, Philadelphia, PA (US); Robert V. Kolakowski, Philadelphia, PA (US); Steven G. Nadler, Philadelphia, PA (US); Karyn T. O'Neil, Philadelphia, PA (US); Yao Xin, Philadelphia, PA (US)

(21) Appl. No.: 18/675,958

(22) Filed: May 28, 2024

Related U.S. Application Data
(62) Division of application No. 17/720,422, filed on Apr. 14, 2022, now Pat. No. 12,037,379.
(60) Provisional application No. 63/174,752, filed on Apr. 14, 2021.
(60) Provisional application No. 63/324,431, filed on Mar. 28, 2022.

(51) Int. Cl. C07K 14/78; A61K 31/7125; A61K 47/64; C12N 15/113

(52) U.S. Cl. CPC C07K 14/78; A61K 31/7125; A61K 47/6435; C12N 15/113; C12N 2310/11; C12N 2310/14; C12N 2310/3513; C12N 2320/32

(57) ABSTRACT

The present disclosure relates to polypeptides, such as fibronectin type III (FN3) domains that can bind CD71, their conjugates, isolated nucleotides encoding the molecules, vectors, host-cells, as well as methods of making and using the same.

### RELATED APPLICATIONS

The present application is a continuation of U.S. Non-Provisional application Ser. No. 17/720,422, filed Apr. 14, 2022, which claims priority to U.S. Provisional Patent Application No. 63/174,752, filed Apr. 14, 2021, and U.S. Provisional Application No. 63/324,431, filed Mar. 28, 2022, which are hereby incorporated by reference in their entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on May 27, 2024, is named “ROO-020USC1.xml” and is 300,680 bytes in size.

### FIELD

The present embodiments relate to fibronectin type III domains (FN3) that specifically bind cluster of differentiation 71 (CD71) and methods of making and using the molecules.

### BACKGROUND

CD71, also known as transferrin receptor 1, is transmembrane that is essential for iron transport into cells. It is highly expressed on many tumor types and at the blood brain barrier, and has thus become an important target for drug delivery. Following binding to iron loaded transferrin, CD71 is rapidly endocytosed and efficiently recycled back to the cell surface. Studies with CD71 antibody drug conjugates suggest that targeting CD71 can improve specificity and selectivity of drug delivery and widen the therapeutic index. In addition, studies using anti-CD71 monoclonal antibodies indicate that binding affinity can play an important role in enabling tissue specific delivery including smooth or skeletal muscle delivery and blood brain barrier transcytosis. Antibodies with high affinity for CD71 are rapidly internalized and alter normal receptor trafficking so that instead of recycling, the receptor is targeted to the lysosome for degradation. In contrast, antibodies with low affinity for CD71 allow for receptor recycling and higher brain exposure.

While antibodies or antibody fragments are the most widely used class of therapeutic proteins when high affinity and specificity for a target molecule are desired, non-antibody proteins can be engineered to also bind such targets. These “alternative scaffold” proteins have advantages over traditional antibodies due to their small size, lack of disulphide bonds, high stability, ability to be expressed in prokaryotic hosts, easy purification, and they are easily conjugated to drugs/toxins, penetrate efficiently into tissues and are readily formatted into multispecific binders.

One such alternative scaffold is the immunoglobulin (Ig) fold. This fold is found in the variable regions of antibodies, as well as thousands of non-antibody proteins. It has been shown that one such Ig protein, the tenth fibronectin type III (FN3) repeat from human fibronectin, can tolerate a number of mutations in surface exposed loops while retaining the overall Ig-fold structure. Thus, what is needed is a FN3 domain that can specifically bind to CD71, and methods of using such molecules for novel therapeutics that enable intracellular access via receptor mediated internalization of CD71.

### SUMMARY

In some embodiments, FN3 domains (e.g. polypeptides) that specifically bind CD71 protein are provided. In some embodiments, the FN3 domains are isolated. In some embodiments, the FN3 domains are recombinant. In some embodiments, the FN3 domains are non-naturally occurring.

In some embodiments, FN3 domains are provided that comprise the amino acid sequence of SEQ ID NOs: 1-7, 10, 12-219, 221-272, 292-299, or 304-306, 292-299, or 304-306, or 304-306. In some embodiments, the FN3 domains bind to CD71. In some embodiments, the FN3 domain binds to human CD71 at a site on CD71 that does not compete with transferrin binding to CD71. In some embodiments, the FN3 domains specifically bind to CD71. In some embodiments, the polypeptide is provided that comprises more than one FN3 domain connected by a linker, such as a flexible linker. In some embodiments, the polypeptide comprises 2, 3, or 4 FN3 domains that are connected to one another by one or more linkers between the domains.

In some embodiments, isolated polynucleotides encoding the FN3 domains described herein are provided.

In some embodiments, a vector comprising the polynucleotides described herein are provided.

In some embodiments, a host cell comprising the vectors described herein are provided.

In some embodiments, methods of producing the FN3 domains are provided. In some embodiments, the method comprises culturing a host cell comprising a vector encoding or expressing the FN3 domain. In some embodiments, the method further comprises purifying the FN3 domain. In some embodiments, the FN3 domain specifically binds CD71.

In some embodiments, pharmaceutical compositions comprising a FN3 domain that binds to CD71 and a pharmaceutically acceptable carrier are provided.

In some embodiments, anti-idiotypic antibodies that binds a FN3 domain that binds to CD71 are provided.

In some embodiments, kits comprising one or more of the FN3 domains are provided.

In some embodiments, methods of detecting CD71-expressing cancer cells in a tumor tissue are provided. In some embodiments, the method comprises obtaining a sample of the tumor tissue from a subject and detecting whether CD71 protein is expressed in the tumor tissue by contacting the sample of the tumor tissue with the FN3 domain that binds CD71 protein comprising the amino acid sequence of one of SEQ ID NOs: 1-7, 10, 12-219, 221-272, 292-299, or 304-306, or 304-306 and detecting the binding between CD71 protein and the FN3 domain.

In some embodiments, methods of isolating CD71 expressing cells are provided. In some embodiments, the method comprises obtaining a sample from a subject; contacting the sample with the FN3 domain that binds CD71 protein comprising the amino acid sequence of one of SEQ ID NOs: 1-7, 10, 12-219, 221-272, 292-299, or 304-306, or 304-306 and isolating the cells bound to the FN3 domains.

In some embodiments, methods of detecting CD71-expressing cancer cells in a tumor tissue are provided. In some embodiments, the method comprises conjugating the FN3 domain that binds CD71 protein comprising the amino acid sequence of one of SEQ ID NOs: 1-7, 10, 12-219, 221-272, 292-299, or 304-306, or 304-306 to a detectable label to form a conjugate; administering the conjugate to a subject; and visualizing the CD71 expressing cancer cells to which the conjugate is bound.

In some embodiments, methods of treating cancer in a subject in need thereof are provided. In some embodiments, the method comprises administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis.

In some embodiments, the polypeptide that binds to CD71 is directed to a muscle cells. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, methods of treating Pompe disease (GSD2, acid alpha-glucosidase (GAA) deficiency) in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

In some embodiments, the polypeptide that binds to CD71 is directed to immune cells. In some embodiments, the polypeptide that binds to CD71 is directed to dendritic cells, T-cells, NK cells, or B-cells. In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, and immune thrombocytopeniaurpura.

In some embodiments, methods of delivering an agent of interest to a CD71 positive cell are provided. In some embodiments, the methods comprise contacting a cell with the agent of interest coupled to a FN3 domain that binds to CD71, such as a polypeptide as provided herein. In some embodiments, the agent of interest is a chemotherapeutic agent, a drug, a growth inhibitory agent, a toxin, a radioactive isotope, an anti-tubulin agent, a polynucleotide, a siRNA molecule, an antisense molecule, a RNA molecule, a DNA molecule, DNA minor groove binders, DNA replication inhibitors, alkylating agents, antibiotics, antifolates, antimetabolites, chemotherapy sensitizers, topoisomerase inhibitors, or a vinca alkaloid.

In some embodiments, the FN3 domains provided for herein are conjugated to a polynucleotide, a siRNA molecule, an antisense molecule, a RNA molecule, or a DNA molecule.

In some embodiments, the polypeptide is a FN3 protein that binds to CD71 at a site that does not compete or inhibit transferrin binding to CD71.

In some embodiments, methods of identifying a FN3 protein that binds to CD71 at a site that does not compete or inhibit transferrin binding to CD71 are provided.

### BRIEF DESCRIPTION OF THE DRAWINGS

FIG. 1 illustrates quantification of AHA1 mRNA in various tissues of CD-1 mice following dosing of FN3 polypeptide (ABX1007) or FN3 polypeptide-siRNA conjugate (ABX1005).

FIG. 2 illustrates dose dependent quantification of AHA1 mRNA in various tissues of C57BL6 mice following dosing of FN3-siRNA conjugate (ABX1005).

FIG. 3 provides the results of a target binding assay using over 6,000 receptors in the proteome array, wherein the data demonstrates that CD71 is the exclusive binding target of the FN3 domain.

FIG. 4 provides data to demonstrate CD71 Centyrin conjugate drives sustained gene knockdown compared with mAb conjugate at 2 weeks, 4 weeks and 8 weeks post dose.

FIG. 5 provides ELISA data demonstrating that Centyrin and Centyrin conjugate actively bind human and cynomolgus monkey CD71.

FIG. 6 provides data demonstrating the ability of Centryin-siRNA AHA1 conjugate to effectively knockdown mRNA levels in vivo in cynomolgus monkey muscles and heart.

### DETAILED DESCRIPTION OF THE DISCLOSURE

As used in this specification and the appended claims, the singular forms “a,” “an,” and “the” include plural referents unless the content clearly dictates otherwise. Thus, for example, reference to “a cell” includes a combination of two or more cells, and the like.

“Fibronectin type III (FN3) domain” (FN3 domain) refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10).

The term “capture agent” refers to substances that bind to a particular type of cells and enable the isolation of that cell from other cells. Exemplary capture agents are magnetic beads, ferrofluids, encapsulating reagents, molecules that bind the particular cell type and the like.

“Sample” refers to a collection of similar fluids, cells, or tissues isolated from a subject, as well as fluids, cells, or tissues present within a subject. Exemplary samples are tissue biopsies, fine needle aspirations, surgically resected tissue, organ cultures, cell cultures and biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium and lavage fluids and the like.

“Substituting” or “substituted” or ‘mutating” or “mutated” refers to altering, deleting of inserting one or more amino acids or nucleotides in a polypeptide or polynucleotide sequence to generate a variant of that sequence.

“Variant” refers to a polypeptide or a polynucleotide that differs from a reference polypeptide or a reference polynucleotide by one or more modifications for example, substitutions, insertions or deletions.

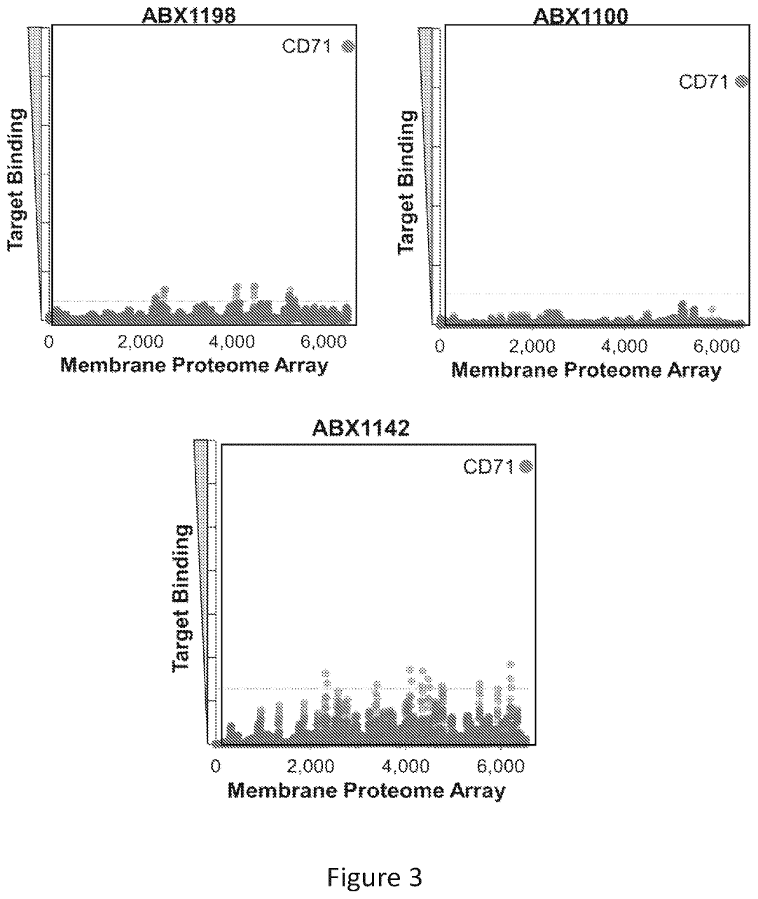

“Specifically binds” or “specific binding” refers to the ability of a FN3 domain to bind to its target, such as CD71, with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Alternatively, “specific binding” refers to the ability of a FN3 domain to bind to its target (e.g. CD71) at least 5-fold above a negative control in standard solution ELISA assay. Specific binding can also be demonstrated using the proteome array as described herein and shown in FIG. 3. In some embodiments, a negative control is an FN3 domain that does not bind CD71. In some embodiment, an FN3 domain that specifically binds CD71 may have cross-reactivity to other related antigens, for example to the same predetermined antigen from other species (homologs), such as Macaca Fascicularis (cynomolgous monkey, cyno) or Pan troglodytes (chimpanzee).

“Library” refers to a collection of variants. The library may be composed of polypeptide or polynucleotide variants.

“Stability” refers to the ability of a molecule to maintain a folded state under physiological conditions such that it retains at least one of its normal functional activities, for example, binding to a predetermined antigen such as CD71.

“CD71” refers to human CD71 protein having the amino acid sequence of SEQ ID NOs: 274 or 275. In some embodiments, SEQ ID NO: 274 is full length human CD71 protein. In some embodiments, SEQ ID NO: 275 is the extracellular domain of human CD71.

SEQ ID NO: 274 = human mature CD71
MTKEYQDLQHLDNEESDHHQLRKGPPPPQPLLQRLCSGPRLLLLSLGLSL
LLLVVVCVIGSQNSQLQEELRGLRETFSNFTASTEAQVKGLSTQGGNVGR
KMKSLESQLEKQQKDLSEDHSSLLLHVKQFVSDLRSLSCQMAALQGNGSE
RTCCPVNWVEHERSCYWFSRSGKAWADADNYCRLEDAHLVVVTSWEEQKF
VQHHIGPVNTWMGLHDQNGPWKWVDGTDYETGFKNWRPEQPDDWYGHGLG
GGEDCAHFTDDGRWNDDVCQRPYRWVCETELDKASQEPPLL
SEQ ID NO: 275 = human mature CD71 extracellular
domain
QNSQLQEELRGLRETFSNFTASTEAQVKGLSTQGGNVGRKMKSLESQLEK
QQKDLSEDHSSLLLHVKQFVSDLRSLSCQMAALQGNGSERTCCPVNWVEH
ERSCYWFSRSGKAWADADNYCRLEDAHLVVVTSWEEQKFVQHHIGPVNTW
MGLHDQNGPWKWVDGTDYETGFKNWRPEQPDDWYGHGLGGGEDCAHFTD
DGRWNDDVCQRPYRWVCETELDKASQEPPLL

“Tencon” refers to the synthetic fibronectin type III (FN3) domain having the sequence shown in SEQ ID NO: 276 and described in U.S. Pat. Publ. No. 2010/0216708.

SEQ ID NO: 276 = Original Tencon Sequence
LPAPKNLVVSEVTEDSLRLSWTAPDAAFDSFLIQYQESEKVGEAINLTVP
GSERSYDLTGLKPGTEYTVSIYGVKGGHRSNPLSAEFTT

A “cancer cell” or a “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

A “dendritic cell” refers to a type of antigen-presenting cell (APC) that form an important role in the adaptive immune system. The main function of dendritic cells is to present antigens. Dendritic cells have the capacity to induce a primary immune response in the inactive or resting naïve T lymphocytes.

An “immune cell” refers to the cells of the immune system categorized as lymphocytes (T-cells, B-cells and NK cells), neutrophils, or monocytes/macrophages. These are all types of white blood cells.

“Vector” refers to a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than about 50 amino acids may be referred to as “peptides”.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Subject” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably.

“Isolated” refers to a homogenous population of molecules (such as synthetic polynucleotides or a polypeptide such as FN3 domains) which have been substantially separated and/or purified away from other components of the system the molecules are produced in, such as a recombinant cell, as well as a protein that has been subjected to at least one purification or isolation step. “Isolated FN3 domain” refers to an FN3 domain that is substantially free of other cellular material and/or chemicals and encompasses FN3 domains that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

### Compositions of Matter

In some embodiments, proteins comprising a polypeptide comprising an amino acid sequence of SEQ ID NOs: 1-7, 10, 12-219, 221-272, 292-299, or 304-306 are provided.

In some embodiments, proteins comprising a polypeptide comprising an amino acid sequence of SEQ ID NO: 273. SEQ ID NO: 273 is a consensus sequence based on the sequences of SEQ ID NO: 288, SEQ ID NO: 289, SEQ ID NO: 290, and SEQ ID NO: 291.

The sequence of SEQ ID NO: 273 is:

MLPAPKNLVVSRVTEDSARLSWTAPDAAFDSFX₁IX₂YX₃EX₄X₅X₆X₇
GEAIX₈LX₉VPGSERSYDLTGLKPGTEYX₁₀VX₁₁IX₁₂X₁₃VKGGX₁₄
X₁₅SX₁₆PLX₁₇AX₁₈FTT

wherein X₈, X₉, X₁₇, and X₁₈ are each, independently, any amino acid other than methionine or proline, and
X₁ is selected from D, F, Y, or H,
X₂ is selected from Y, G, A, or V,
X₃ is selected from I, T, L, A, or H,
X₄ is selected from S, Y or P,
X₅ is selected from Y, G, Q, or R,
X₆ is selected from G or P,
X₇ is selected from A, Y, P, D, or S,
X₁₀ is selected from W, N, S, or E,
X₁₁ is selected from L, Y, or G,
X₁₂ is selected from D, Q, H, or V,
X₁₃ is selected from G or S,
X₁₄ is selected from R, G, F, L, or D,
X₁₅ is selected from W, S, P, or L, and
X₁₆ is selected from T, V, M, or S.

In some embodiments:
X₁ is selected from D, F, Y, or H,
X₂ is selected from G, A, or V,
X₃ is selected from T, L, A, or H,
X₄ is selected from Y or P,
X₅ is selected from G, Q, or R,
X₆ is selected from G or P,
X₇ is selected from Y, P, D, or S,
X₁₀ is selected from W, N, S, or E,
X₁₁ is selected from L, Y, or G,
X₁₂ is selected from Q, H, or V,
X₁₃ is selected from G or S,
X₁₄ is selected from G, F, L, or D,
X₁₅ is selected from S, P, or L, and
X₁₆ is selected from V, M, or S.

In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 288. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 289. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 290. In some embodiments, X₁, X₂, X₃, X₄, X₅, X₆, X₇, X₁₀, X₁₁, X₁₂, X₁₃, X₁₄, X₁₅, and X₁₆ are as shown in the sequence of SEQ ID NO: 291.

In some embodiments, X₈, X₉, X₁₇, and X₁₈ is, independently, alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, not alanine, arginine, asparagine, aspartic acid, cysteine, glutamine, glutamic acid, glycine, histidine, isoleucine, leucine, lysine, phenylalanine, serine, threonine, tryptophan, tyrosine, or valine. In some embodiments, X₈, X₉, X₁₇, and X₁₈ is, independently, alanine. In some embodiments, X₈, X₉, X₁₇, and X₁₈ is, independently, arginine. In some embodiments, X₈, X₉, X₁₇, and X₁₈ is, independently asparagine. In some embodiments, X₈, X₉, X₁₇, and X₁₈ is, independently, aspartic acid. In some embodiments, X₈, X₉, X₁₇, and X₁₈ is, independently, cysteine. In some embodiments, X₈, X₉, X₁₇, and X₁₈ is, independently, glutamine. In some embodiments, X₈, X₉, X₁₇, and X₁₈ is, independently, glutamic acid. In some embodiments, X₈, X₉, X₁₇, and X₁₈ is, independently, glycine. In some embodiments, X₈, X₉, X₁₇, and X₁₈ is, independently, histidine. In some embodiments, X₈, X₉, X₁₇, and X₁₈ is, independently, isoleucine. In some embodiments, X₈, X₉, X₁₇, and X₁₈ is, independently, leucine. In some embodiments, X₈, X₉, X₁₇, and X₁₈ is, independently, lysine. In some embodiments, X₈, X₉, X₁₇, and X₁₈ is, independently, phenylalanine. In some embodiments, X₈, X₉, X₁₇, and X₁₈ is, independently serine. In some embodiments, X₈, X₉, X₁₇, and X₁₈ is, independently, threonine. In some embodiments, X₈, X₉, X₁₇, and X₁₈ is, independently, tryptophan. In some embodiments, X₈, X₉, X₁₇, and X₁₈ is, independently, tyrosine. In some embodiments, X₈, X₉, X₁₇, and X₁₈ is, independently valine.

In some embodiments, the sequence is set forth as shown in in the sequence of SEQ ID NO: 288, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 289, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 290, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, the sequence is set forth as shown in in the sequence of SEQ ID NO: 291, except that the positions that correspond to the positions of X₈, X₉, X₁₇, and X₁₈ can be any other amino acid residue as set forth above, except that in some embodiments, X₈ is not V, X₉ is not T, X₁₇ is not S, and X₁₈ is not I.

In some embodiments, proteins comprising a polypeptide comprising an amino acid sequence that is at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to a sequence of SEQ ID NO: 273. In some embodiments, the protein is at least 85%, 86%, 87%, 88%, 89%, 90%, 91%, 92%, 93%, 94%, 95%, 96%, 97%, 98% or 99% identical to a sequence of SEQ ID NO: 273. In some embodiments, the protein is at least 90%, 91%, 92%, 93%, 94%, 95%, 96%, 97%, 98% or 99% identical to a sequence of SEQ ID NO: 273. In some embodiments, the protein is at least 95%, 96%, 97%, 98% or 99% identical to a sequence of SEQ ID NO: 273. In some embodiments, the protein or polypeptide is at least 70%, 75%, 80%, 85%, or 90% identical to SEQ ID NO: 273.

Percent identity can be determined using the default parameters to align two sequences using BlastP available through the NCBI website.

The polypeptides provided herein can be part of a larger polypeptide and can be referred to as a domain. The homology or identity between two domains in different polypeptides is based on the domains that are similar as opposed to the overall polypeptide. For example, if a polypeptide comprises a polypeptide comprising a FN3 domain comprising SEQ ID NO: 1 and said domain is conjugated to a scFV antibody, another protein that has a domain that is similar but not identical to SEQ ID NO: 1 can be at least 90% identical even if the scFV shares no homology. Thus, the % identity can be based on the domain or on the entire length of the polypeptide. Methods of determining % identity are provided for herein or are known to one of skill in the art.

In some embodiments, fibronectin type III (FN3) domains that bind or specifically bind human CD71 protein (SEQ ID Nos: 274 or 275) are provided. As provided herein, the FN3 domains can bind to the CD71 protein. Also provided, even if not explicitly stated is that the domains can also specifically bind to the CD71 protein. Thus, for example, a FN3 domain that binds to CD71 would also encompass a FN3 domain protein that specifically binds to CD71. These molecules can be used, for example, in therapeutic and diagnostic applications and in imaging. In some embodiments, polynucleotides encoding the FN3 domains disclosed herein or complementary nucleic acids thereof, vectors, host cells, and methods of making and using them are provided.

In some embodiments, an isolated FN3 domain that binds or specifically binds CD71 is provided.

In some embodiments, the FN3 domain comprises two FN3 domains connected by a linker. The linker can be a flexible linker. The linker can be a short peptide sequence, such as those described herein. For example, the linker can be a G/S linker and the like.

In some embodiments, the FN3 domain comprising two FN3 domains connected by a linker, such as those provided for herein. Exemplary linker include, but are not limited to, (GS)2, (SEQ ID NO: 278), (GGGS)2 (SEQ ID NO: 279), (GGGGS)1-5 (SEQ ID NO: 280), (AP)1-20 (SEQ ID NO: 311); (AP)2 (SEQ ID NO: 281), (AP)5 (SEQ ID NO: 282), (AP)₁₀ (SEQ ID NO: 283), (AP)₂₀ (SEQ ID NO: 284), A(EAAAK)₅AAA (SEQ ID NO: 285), or (EAAAK)₁₋₅ (SEQ ID NO: 307). In some embodiments, the linker comprises or is an amino acid sequence of: EAAAKEAAAKEAAAKEAAAK (SEQ ID NO: 300); GGGGSGGGGSGGGGSGGGGS (SEQ ID NO: 301); APAPAPAPAP (SEQ ID NO: 302); or EAAAK (SEQ ID NO: 303).

In some embodiments, the polypeptide comprises an amino acid sequence of SEQ ID NO: 292. In some embodiments, the polypeptide comprises an amino acid sequence of SEQ ID NO: 293. In some embodiments, the polypeptide comprises an amino acid sequence of SEQ ID NO: 294. In some embodiments, the polypeptide comprises an amino acid sequence of SEQ ID NO: 295. In some embodiments, the polypeptide comprises an amino acid sequence of SEQ ID NO: 296. In some embodiments, the polypeptide comprises an amino acid sequence of SEQ ID NO: 297. In some embodiments, the polypeptide comprises an amino acid sequence of SEQ ID NO: 298. In some embodiments, the polypeptide comprises an amino acid sequence of SEQ ID NO: 299.

In some embodiments, the FN3 domain may bind CD71 with a dissociation constant (K_{D}) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., KD, Kon, Koff) are made with standardized solutions of protein scaffold and antigen, and a standardized buffer, such as the buffers described herein.

In some embodiments, the FN3 domain may bind CD71 at least 5-fold above the signal obtained for a negative control in a standard solution ELISA assay.

In some embodiments, the FN3 domain that binds or specifically binds CD71 comprises an initiator methionine (Met) linked to the N-terminus of the molecule. In some embodiments, the FN3 domain that binds or specifically binds CD71 comprises a cysteine (Cys) linked to a C-terminus of the FN3 domain. The addition of the N-terminal Met and/or the C-terminal Cys may facilitate expression and/or conjugation of half-life extending molecules.

The FN3 domain can also contain cysteine substitutions, such as those that are described in U.S. Pat. No. 10,196,446, which is hereby incorporated by reference in its entirety. Briefly, in some embodiments, the polypeptides provided herein can comprise at least one cysteine substitution at a position selected from the group consisting of residues 6, 8, 10, 11, 14, 15, 16, 20, 30, 34, 38, 40, 41, 45, 47, 48, 53, 54, 59, 60, 62, 64, 70, 88, 89, 90, 91, and 93 of the FN3 domain based on SEQ ID NO: 6 or SEQ ID NO: 1 of U.S. Pat. No. 10,196,446, and the equivalent positions in related FN3 domains. In some embodiments, the substitution is at residue 6. In some embodiments, the substitution is at residue 8. In some embodiments, the substitution is at residue 10. In some embodiments, the substitution is at residue 11. In some embodiments, the substitution is at residue 14. In some embodiments, the substitution is at residue 15. In some embodiments, the substitution is at residue 16. In some embodiments, the substitution is at residue 20. In some embodiments, the substitution is at residue 30. In some embodiments, the substitution is at residue 34. In some embodiments, the substitution is at residue 38. In some embodiments, the substitution is at residue 40. In some embodiments, the substitution is at residue 41. In some embodiments, the substitution is at residue 45. In some embodiments, the substitution is at residue 47. In some embodiments, the substitution is at residue 48. In some embodiments, the substitution is at residue 53. In some embodiments, the substitution is at residue 54. In some embodiments, the substitution is at residue 59. In some embodiments, the substitution is at residue 60. In some embodiments, the substitution is at residue 62. In some embodiments, the substitution is at residue 64. In some embodiments, the substitution is at residue 70. In some embodiments, the substitution is at residue 88. In some embodiments, the substitution is at residue 89. In some embodiments, the substitution is at residue 90. In some embodiments, the substitution is at residue 91. In some embodiments, the substitution is at residue 93.

A cysteine substitution at a position in the domain or protein comprises a replacement of the existing amino acid residue with a cysteine residue. Other examples of cysteine modifications can be found in, for example, U.S. Patent Application Publication No. 20170362301, which is hereby incorporated by reference in its entirety. The alignment of the sequences can be performed using BlastP using the default parameters at, for example, the NCBI website.

In some embodiments, the FN3 domain that binds CD71 is internalized into a cell. In some embodiments, internalization of the FN3 domain may facilitate delivery of a detectable label or therapeutic into a cell. In some embodiments, internalization of the FN3 domain may facilitate delivery of a cytotoxic agent into a cell. The cytotoxic agent can act as a therapeutic agent. In some embodiments, internalization of the FN3 domain may facilitate the delivery of any detectable label, therapeutic, and/or cytotoxic agent disclosed herein into a cell. In some embodiments, the cell is a tumor cell. In some embodiments, the cell is a liver cell. In some embodiments, the cell is a muscle cell. In some embodiments, the cell is an immune cell. In some embodiments, the cell is a dendritic cell. In some embodiments, the cell is a T-cell. In some embodiments, the cell is a NK cells. In some embodiments, the cell is a B-cell. In some embodiments, the cell is a cell of the central nervous system.

In some embodiments, the FN3 domain that binds CD71 is based on Tencon sequence of SEQ ID NO: 276 or Tencon 27 sequence of SEQ ID NO: 277, optionally having substitutions at residues positions 11, 14, 17, 37, 46, 73, or 86 (residue numbering corresponding to SEQ ID NO: 277).

SEQ ID NO: 276 = Original Tencon Sequence
LPAPKNLVVSEVTEDSLRLSWTAPDAAFDSFLIQYQESEKVGEAINLTVP
GSERSYDLTGLKPGTEYTVSIYGVKGGHRSNPLSAEFTT
SEQ ID NO: 277 = Stabilized Tencon (Tencon27)
LPAPKNLVVSRVTEDSARLSWTAPDAAFDSFLIQYQESEKVGEAIVLTVP
GSERSYDLTGLKPGTEYTVSIYGVKGGHRSNPLSAIFTT

In some embodiments, an isolated FN3 domain that binds CD71 comprises the amino acid sequence of SEQ ID Nos: 1-7, 10, 12-219, 221-272, 292-299, or 304-306.

In some embodiments, an isolated FN3 domain that binds CD71 comprises the amino acid sequence of SEQ ID NO: 1. In some embodiments, an isolated FN3 domain that binds CD71 comprises the amino acid sequence of SEQ ID NO: 2. In some embodiments, an isolated FN3 domain that binds CD71 comprises the amino acid sequence of SEQ ID NO: 3. In some embodiments, an isolated FN3 domain that binds CD71 comprises the amino acid sequence of SEQ ID NO: 4. In some embodiments, an isolated FN3 domain that binds CD71 comprises the amino acid sequence of SEQ ID NO: 5. In some embodiments, an isolated FN3 domain that binds CD71 comprises the amino acid sequence of SEQ ID NO: 6. In some embodiments, an isolated FN3 domain that binds CD71 comprises the amino acid sequence of SEQ ID NO: 7. In some embodiments, an isolated FN3 domain that binds CD71 comprises the amino acid sequence of SEQ ID NO: 10. In some embodiments, an isolated FN3 domain that binds CD71 comprises the amino acid sequence of SEQ ID NO: 12. In some embodiments, an isolated FN3 domain that binds CD71 comprises the amino acid sequence of SEQ ID NO: 13. In some embodiments, an isolated FN3 domain that binds CD71 comprises the amino acid sequence of SEQ ID NO: 14. In some embodiments, an isolated FN3 domain that binds CD71 comprises the amino acid sequence of SEQ ID NO: 15. In some embodiments, an isolated FN3 domain that binds CD71 comprises the amino acid sequence of SEQ ID NO: 16. In some embodiments, an isolated FN3 domain that binds CD71 comprises the amino acid sequence of SEQ ID NO: 17. In some embodiments, an isolated FN3 domain that binds CD71 comprises the amino acid sequence of SEQ ID NO: 18. In some embodiments, an isolated FN3 domain that binds CD71 comprises the amino acid sequence of SEQ ID NO: 19. In some embodiments, an isolated FN3 domain that binds CD71 comprises the amino acid sequence of SEQ ID NO: 20. In some embodiments, an isolated FN3 domain that binds CD71 comprises the amino acid sequence of SEQ ID NO: 21. In some embodiments, an isolated FN3 domain that binds CD71 comprises the amino acid sequence of SEQ ID NO: 22. In some embodiments, an isolated FN3 domain that binds CD71 comprises the amino acid sequence of SEQ ID NO: 23. In some embodiments, an isolated FN3 domain that binds CD71 comprises the amino acid sequence of SEQ ID NO: 24. In some embodiments, an isolated FN3 domain that binds CD71 comprises the amino acid sequence of SEQ ID NO: 25. In some embodiments, an isolated FN3 domain that binds CD71 comprises the amino acid sequence of SEQ ID NO: 26. In some embodiments, an isolated FN3 domain that binds CD71 comprises the amino acid sequence of SEQ ID NO: 27. In some embodiments, an isolated FN3 domain that binds CD71 comprises the amino acid sequence of SEQ ID NO: 28. In some embodiments, an isolated FN3 domain that binds CD71 comprises the amino acid sequence of SEQ ID NO: 29. In some embodiments, an isolated FN3 domain that binds CD71 comprises the amino acid sequence of SEQ ID NO: 30. In some embodiments, an isolated FN3 domain that binds CD71 comprises the amino acid sequence of SEQ ID NO: 31. In some embodiments, an isolated FN3 domain that binds CD71 comprises the amino acid sequence of SEQ ID NO: 32. In some embodiments, an isolated FN3 domain that binds CD71 comprises the amino acid sequence of SEQ ID NO: 33. In some embodiments, an isolated FN3 domain that binds CD71 comprises the amino acid sequence of SEQ ID NO: 34. In some embodiments, an isolated FN3 domain that binds CD71 comprises the amino acid sequence of SEQ ID NO: 35. In some embodiments, an isolated FN3 domain that binds CD71 comprises the amino acid sequence of SEQ ID NO: 36. In some embodiments, an isolated FN3 domain that binds CD71 comprises the amino acid sequence of SEQ ID NO: 37. In some embodiments, an isolated FN3 domain that binds CD71 comprises the amino acid sequence of SEQ ID NO: 38. In some embodiments, an isolated FN3 domain that binds CD71 comprises the amino acid sequence of SEQ ID NO: 39. In some embodiments, an isolated FN3 domain that binds CD71 comprises the amino acid sequence of SEQ ID NO: 40. In some embodiments, an isolated FN3 domain that binds CD71 comprises the amino acid sequence of SEQ ID NO: 41. In some embodiments, an isolated FN3 domain that binds CD71 comprises the amino acid sequence of SEQ ID NO: 42. In some embodiments, an isolated FN3 domain that binds CD71 comprises the amino acid sequence of SEQ ID NO: 43. In some embodiments, an isolated FN3 domain that binds CD71 comprises the amino acid sequence of SEQ ID NO: 44. In some embodiments, an isolated FN3 domain that binds CD71 comprises the amino acid sequence of SEQ ID NO: 45. In some embodiments, an isolated FN3 domain that binds CD71 comprises the amino acid sequence of SEQ ID NO: 46. In some embodiments, an isolated FN3 domain that binds CD71 comprises the amino acid sequence of SEQ ID NO: 47. In some embodiments, an isolated FN3 domain that binds CD71 comprises the amino acid sequence of SEQ ID NO: 48. In some embodiments, an isolated FN3 domain that binds CD71 comprises the amino acid sequence of SEQ ID NO: 49. In some embodiments, an isolated FN3 domain that binds CD71 comprises the amino acid sequence of SEQ ID NO: 50. In some embodiments, an isolated FN3 domain that binds CD71 comprises the amino acid sequence of SEQ ID NO: 51. In some embodiments, an isolated FN3 domain that binds CD71 comprises the amino acid sequence of SEQ ID NO: 52. In some embodiments, an isolated FN3 domain that binds CD71 comprises the amino acid sequence of SEQ ID NO: 53. In some embodiments, an isolated FN3 domain that binds CD71 comprises the amino acid sequence of SEQ ID NO: 54. In some embodiments, an isolated FN3 domain that binds CD71 comprises the amino acid sequence of SEQ ID NO: 55. In some embodiments, an isolated FN3 domain that binds CD71 comprises the amino acid sequence of SEQ ID NO: 56. In some embodiments, an isolated FN3 domain that binds CD71 comprises the amino acid sequence of SEQ ID NO: 57. In some embodiments, an isolated FN3 domain that binds CD71 comprises the amino acid sequence of SEQ ID NO: 58. In some embodiments, an isolated FN3 domain that binds CD71 comprises the amino acid sequence of SEQ ID NO: 59. In some embodiments, an isolated FN3 domain that binds CD71 comprises the amino acid sequence of SEQ ID NO: 60. In some embodiments, an isolated FN3 domain that binds CD71 comprises the amino acid sequence of SEQ ID NO: 61. In some embodiments, an isolated FN3 domain that binds CD71 comprises the amino acid sequence of SEQ ID NO: 62. In some embodiments, an isolated FN3 domain that binds CD71 comprises the amino acid sequence of SEQ ID NO: 63. In some embodiments, an isolated FN3 domain that binds CD71 comprises the amino acid sequence of SEQ ID NO: 64. In some embodiments, an isolated FN3 domain that binds CD71 comprises the amino acid sequence of SEQ ID NO: 65. In some embodiments, an isolated FN3 domain that binds CD71 comprises the amino acid sequence of SEQ ID NO: 66. In some embodiments, an isolated FN3 domain that binds CD71 comprises the amino acid sequence of SEQ ID NO: 67. In some embodiments, an isolated FN3 domain that binds CD71 comprises the amino acid sequence of SEQ ID NO: 68. In some embodiments, an isolated FN3 domain that binds CD71 comprises the amino acid sequence of SEQ ID NO: 69. In some embodiments, an isolated FN3 domain that binds CD71 comprises the amino acid sequence of SEQ ID NO: 70. In some embodiments, an isolated FN3 domain that binds CD71 comprises the amino acid sequence of SEQ ID NO: 71. In some embodiments, an isolated FN3 domain that binds CD71 comprises the amino acid sequence of SEQ ID NO: 72. In some embodiments, an isolated FN3 domain that binds CD71 comprises the amino acid sequence of SEQ ID NO: 73. In some embodiments, an isolated FN3 domain that binds CD71 comprises the amino acid sequence of SEQ ID NO: 74. In some embodiments, an isolated FN3 domain that binds CD71 comprises the amino acid sequence of SEQ ID NO: 75. In some embodiments, an isolated FN3 domain that binds CD71 comprises the amino acid sequence of SEQ ID NO: 76. In some embodiments, an isolated FN3 domain that binds CD71 comprises the amino acid sequence of SEQ ID NO: 77. In some embodiments, an isolated FN3 domain that binds CD71 comprises the amino acid sequence of SEQ ID NO: 78. In some embodiments, an isolated FN3 domain that binds CD71 comprises the amino acid sequence of SEQ ID NO: 79. In some embodiments, an isolated FN3 domain that binds CD71 comprises the amino acid sequence of SEQ ID NO: 80. In some embodiments, an isolated FN3 domain that binds CD71 comprises the amino acid sequence of SEQ ID NO: 81. In some embodiments, an isolated FN3 domain that binds CD71 comprises the amino acid sequence of SEQ ID NO: 82. In some embodiments, an isolated FN3 domain that binds CD71 comprises the amino acid sequence of SEQ ID NO: 83. In some embodiments, an isolated FN3 domain that binds CD71 comprises the amino acid sequence of SEQ ID NO: 84. In some embodiments, an isolated FN3 domain that binds CD71 comprises the amino acid sequence of SEQ ID NO: 85. In some embodiments, an isolated FN3 domain that binds CD71 comprises the amino acid sequence of SEQ ID NO: 86. In some embodiments, an isolated FN3 domain that binds CD71 comprises the amino acid sequence of SEQ ID NO: 87. In some embodiments, an isolated FN3 domain that binds CD71 comprises the amino acid sequence of SEQ ID NO: 88. In some embodiments, an isolated FN3 domain that binds CD71 comprises the amino acid sequence of SEQ ID NO: 89. In some embodiments, an isolated FN3 domain that binds CD71 comprises the amino acid sequence of SEQ ID NO: 90. In some embodiments, an isolated FN3 domain that binds CD71 comprises the amino acid sequence of SEQ ID NO: 91. In some embodiments, an isolated FN3 domain that binds CD71 comprises the amino acid sequence of SEQ ID NO: 92. In some embodiments, an isolated FN3 domain that binds CD71 comprises the amino acid sequence of SEQ ID NO: 93. In some embodiments, an isolated FN3 domain that binds CD71 comprises the amino acid sequence of SEQ ID NO: 94. In some embodiments, an isolated FN3 domain that binds CD71 comprises the amino acid sequence of SEQ ID NO: 95. In some embodiments, an isolated FN3 domain that binds CD71 comprises the amino acid sequence of SEQ ID NO: 96. In some embodiments, an isolated FN3 domain that binds CD71 comprises the amino acid sequence of SEQ ID NO: 97. In some embodiments, an isolated FN3 domain that binds CD71 comprises the amino acid sequence of SEQ ID NO: 98. In some embodiments, an isolated FN3 domain that binds CD71 comprises the amino acid sequence of SEQ ID NO: 99. In some embodiments, an isolated FN3 domain that binds CD71 comprises the amino acid sequence of SEQ ID NO: 100. In some embodiments, an isolated FN3 domain that binds CD71 comprises the amino acid sequence of SEQ ID NO: 101. In some embodiments, an isolated FN3 domain that binds CD71 comprises the amino acid sequence of SEQ ID NO: 102. In some embodiments, an isolated FN3 domain that binds CD71 comprises the amino acid sequence of SEQ ID NO: 103. In some embodiments, an isolated FN3 domain that binds CD71 comprises the amino acid sequence of SEQ ID NO: 104. In some embodiments, an isolated FN3 domain that binds CD71 comprises the amino acid sequence of SEQ ID NO: 105. In some embodiments, an isolated FN3 domain that binds CD71 comprises the amino acid sequence of SEQ ID NO: 106. In some embodiments, an isolated FN3 domain that binds CD71 comprises the amino acid sequence of SEQ ID NO: 107. In some embodiments, an isolated FN3 domain that binds CD71 comprises the amino acid sequence of SEQ ID NO: 108. In some embodiments, an isolated FN3 domain that binds CD71 comprises the amino acid sequence of SEQ ID NO: 109. In some embodiments, an isolated FN3 domain that binds CD71 comprises the amino acid sequence of SEQ ID NO: 110. In some embodiments, an isolated FN3 domain that binds CD71 comprises the amino acid sequence of SEQ ID NO: 111. In some embodiments, an isolated FN3 domain that binds CD71 comprises the amino acid sequence of SEQ ID NO: 112. In some embodiments, an isolated FN3 domain that binds CD71 comprises the amino acid sequence of SEQ ID NO: 113. In some embodiments, an isolated FN3 domain that binds CD71 comprises the amino acid sequence of SEQ ID NO: 114. In some embodiments, an isolated FN3 domain that binds CD71 comprises the amino acid sequence of SEQ ID NO: 115. In some embodiments, an isolated FN3 domain that binds CD71 comprises the amino acid sequence of SEQ ID NO: 116. In some embodiments, an isolated FN3 domain that binds CD71 comprises the amino acid sequence of SEQ ID NO: 117. In some embodiments, an isolated FN3 domain that binds CD71 comprises the amino acid sequence of SEQ ID NO: 118. In some embodiments, an isolated FN3 domain that binds CD71 comprises the amino acid sequence of SEQ ID NO: 119. In some embodiments, an isolated FN3 domain that binds CD71 comprises the amino acid sequence of SEQ ID NO: 120. In some embodiments, an isolated FN3 domain that binds CD71 comprises the amino acid sequence of SEQ ID NO: 121. In some embodiments, an isolated FN3 domain that binds CD71 comprises the amino acid sequence of SEQ ID NO: 122. In some embodiments, an isolated FN3 domain that binds CD71 comprises the amino acid sequence of SEQ ID NO: 123. In some embodiments, an isolated FN3 domain that binds CD71 comprises the amino acid sequence of SEQ ID NO: 124. In some embodiments, an isolated FN3 domain that binds CD71 comprises the amino acid sequence of SEQ ID NO: 125. In some embodiments, an isolated FN3 domain that binds CD71 comprises the amino acid sequence of SEQ ID NO: 126. In some embodiments, an isolated FN3 domain that binds CD71 comprises the amino acid sequence of SEQ ID NO: 127. In some embodiments, an isolated FN3 domain that binds CD71 comprises the amino acid sequence of SEQ ID NO: 128. In some embodiments, an isolated FN3 domain that binds CD71 comprises the amino acid sequence of SEQ ID NO: 129. In some embodiments, an isolated FN3 domain that binds CD71 comprises the amino acid sequence of SEQ ID NO: 130. In some embodiments, an isolated FN3 domain that binds CD71 comprises the amino acid sequence of SEQ ID NO: 131. In some embodiments, an isolated FN3 domain that binds CD71 comprises the amino acid sequence of SEQ ID NO: 132. In some embodiments, an isolated FN3 domain that binds CD71 comprises the amino acid sequence of SEQ ID NO: 133. In some embodiments, an isolated FN3 domain that binds CD71 comprises the amino acid sequence of SEQ ID NO: 134. In some embodiments, an isolated FN3 domain that binds CD71 comprises the amino acid sequence of SEQ ID NO: 135. In some embodiments, an isolated FN3 domain that binds CD71 comprises the amino acid sequence of SEQ ID NO: 136. In some embodiments, an isolated FN3 domain that binds CD71 comprises the amino acid sequence of SEQ ID NO: 137. In some embodiments, an isolated FN3 domain that binds CD71 comprises the amino acid sequence of SEQ ID NO: 138. In some embodiments, an isolated FN3 domain that binds CD71 comprises the amino acid sequence of SEQ ID NO: 139. In some embodiments, an isolated FN3 domain that binds CD71 comprises the amino acid sequence of SEQ ID NO: 140. In some embodiments, an isolated FN3 domain that binds CD71 comprises the amino acid sequence of SEQ ID NO: 141. In some embodiments, an isolated FN3 domain that binds CD71 comprises the amino acid sequence of SEQ ID NO: 142. In some embodiments, an isolated FN3 domain that binds CD71 comprises the amino acid sequence of SEQ ID NO: 143. In some embodiments, an isolated FN3 domain that binds CD71 comprises the amino acid sequence of SEQ ID NO: 144. In some embodiments, an isolated FN3 domain that binds CD71 comprises the amino acid sequence of SEQ ID NO: 145. In some embodiments, an isolated FN3 domain that binds CD71 comprises the amino acid sequence of SEQ ID NO: 146. In some embodiments, an isolated FN3 domain that binds CD71 comprises the amino acid sequence of SEQ ID NO: 147. In some embodiments, an isolated FN3 domain that binds CD71 comprises the amino acid sequence of SEQ ID NO: 148. In some embodiments, an isolated FN3 domain that binds CD71 comprises the amino acid sequence of SEQ ID NO: 149. In some embodiments, an isolated FN3 domain that binds CD71 comprises the amino acid sequence of SEQ ID NO: 150. In some embodiments, an isolated FN3 domain that binds CD71 comprises the amino acid sequence of SEQ ID NO: 151. In some embodiments, an isolated FN3 domain that binds CD71 comprises the amino acid sequence of SEQ ID NO: 152. In some embodiments, an isolated FN3 domain that binds CD71 comprises the amino acid sequence of SEQ ID NO: 153. In some embodiments, an isolated FN3 domain that binds CD71 comprises the amino acid sequence of SEQ ID NO: 154. In some embodiments, an isolated FN3 domain that binds CD71 comprises the amino acid sequence of SEQ ID NO: 155. In some embodiments, an isolated FN3 domain that binds CD71 comprises the amino acid sequence of SEQ ID NO: 156. In some embodiments, an isolated FN3 domain that binds CD71 comprises the amino acid sequence of SEQ ID NO: 157. In some embodiments, an isolated FN3 domain that binds CD71 comprises the amino acid sequence of SEQ ID NO: 158. In some embodiments, an isolated FN3 domain that binds CD71 comprises the amino acid sequence of SEQ ID NO: 159. In some embodiments, an isolated FN3 domain that binds CD71 comprises the amino acid sequence of SEQ ID NO: 160. In some embodiments, an isolated FN3 domain that binds CD71 comprises the amino acid sequence of SEQ ID NO: 161. In some embodiments, an isolated FN3 domain that binds CD71 comprises the amino acid sequence of SEQ ID NO: 162. In some embodiments, an isolated FN3 domain that binds CD71 comprises the amino acid sequence of SEQ ID NO: 163. In some embodiments, an isolated FN3 domain that binds CD71 comprises the amino acid sequence of SEQ ID NO: 164. In some embodiments, an isolated FN3 domain that binds CD71 comprises the amino acid sequence of SEQ ID NO: 165. In some embodiments, an isolated FN3 domain that binds CD71 comprises the amino acid sequence of SEQ ID NO: 166. In some embodiments, an isolated FN3 domain that binds CD71 comprises the amino acid sequence of SEQ ID NO: 167. In some embodiments, an isolated FN3 domain that binds CD71 comprises the amino acid sequence of SEQ ID NO: 168. In some embodiments, an isolated FN3 domain that binds CD71 comprises the amino acid sequence of SEQ ID NO: 169. In some embodiments, an isolated FN3 domain that binds CD71 comprises the amino acid sequence of SEQ ID NO: 170. In some embodiments, an isolated FN3 domain that binds CD71 comprises the amino acid sequence of SEQ ID NO: 171. In some embodiments, an isolated FN3 domain that binds CD71 comprises the amino acid sequence of SEQ ID NO: 172. In some embodiments, an isolated FN3 domain that binds CD71 comprises the amino acid sequence of SEQ ID NO: 173. In some embodiments, an isolated FN3 domain that binds CD71 comprises the amino acid sequence of SEQ ID NO: 174. In some embodiments, an isolated FN3 domain that binds CD71 comprises the amino acid sequence of SEQ ID NO: 175. In some embodiments, an isolated FN3 domain that binds CD71 comprises the amino acid sequence of SEQ ID NO: 176. In some embodiments, an isolated FN3 domain that binds CD71 comprises the amino acid sequence of SEQ ID NO: 177. In some embodiments, an isolated FN3 domain that binds CD71 comprises the amino acid sequence of SEQ ID NO: 178. In some embodiments, an isolated FN3 domain that binds CD71 comprises the amino acid sequence of SEQ ID NO: 179. In some embodiments, an isolated FN3 domain that binds CD71 comprises the amino acid sequence of SEQ ID NO: 180. In some embodiments, an isolated FN3 domain that binds CD71 comprises the amino acid sequence of SEQ ID NO: 181. In some embodiments, an isolated FN3 domain that binds CD71 comprises the amino acid sequence of SEQ ID NO: 182. In some embodiments, an isolated FN3 domain that binds CD71 comprises the amino acid sequence of SEQ ID NO: 183. In some embodiments, an isolated FN3 domain that binds CD71 comprises the amino acid sequence of SEQ ID NO: 184. In some embodiments, an isolated FN3 domain that binds CD71 comprises the amino acid sequence of SEQ ID NO: 185. In some embodiments, an isolated FN3 domain that binds CD71 comprises the amino acid sequence of SEQ ID NO: 186. In some embodiments, an isolated FN3 domain that binds CD71 comprises the amino acid sequence of SEQ ID NO: 187. In some embodiments, an isolated FN3 domain that binds CD71 comprises the amino acid sequence of SEQ ID NO: 188. In some embodiments, an isolated FN3 domain that binds CD71 comprises the amino acid sequence of SEQ ID NO: 189. In some embodiments, an isolated FN3 domain that binds CD71 comprises the amino acid sequence of SEQ ID NO: 190. In some embodiments, an isolated FN3 domain that binds CD71 comprises the amino acid sequence of SEQ ID NO: 191. In some embodiments, an isolated FN3 domain that binds CD71 comprises the amino acid sequence of SEQ ID NO: 192. In some embodiments, an isolated FN3 domain that binds CD71 comprises the amino acid sequence of SEQ ID NO: 193. In some embodiments, an isolated FN3 domain that binds CD71 comprises the amino acid sequence of SEQ ID NO: 194. In some embodiments, an isolated FN3 domain that binds CD71 comprises the amino acid sequence of SEQ ID NO: 195. In some embodiments, an isolated FN3 domain that binds CD71 comprises the amino acid sequence of SEQ ID NO: 196. In some embodiments, an isolated FN3 domain that binds CD71 comprises the amino acid sequence of SEQ ID NO: 197. In some embodiments, an isolated FN3 domain that binds CD71 comprises the amino acid sequence of SEQ ID NO: 198. In some embodiments, an isolated FN3 domain that binds CD71 comprises the amino acid sequence of SEQ ID NO: 199. In some embodiments, an isolated FN3 domain that binds CD71 comprises the amino acid sequence of SEQ ID NO: 200. In some embodiments, an isolated FN3 domain that binds CD71 comprises the amino acid sequence of SEQ ID NO: 201. In some embodiments, an isolated FN3 domain that binds CD71 comprises the amino acid sequence of SEQ ID NO: 202. In some embodiments, an isolated FN3 domain that binds CD71 comprises the amino acid sequence of SEQ ID NO: 203. In some embodiments, an isolated FN3 domain that binds CD71 comprises the amino acid sequence of SEQ ID NO: 204. In some embodiments, an isolated FN3 domain that binds CD71 comprises the amino acid sequence of SEQ ID NO: 205. In some embodiments, an isolated FN3 domain that binds CD71 comprises the amino acid sequence of SEQ ID NO: 206. In some embodiments, an isolated FN3 domain that binds CD71 comprises the amino acid sequence of SEQ ID NO: 207. In some embodiments, an isolated FN3 domain that binds CD71 comprises the amino acid sequence of SEQ ID NO: 208. In some embodiments, an isolated FN3 domain that binds CD71 comprises the amino acid sequence of SEQ ID NO: 209. In some embodiments, an isolated FN3 domain that binds CD71 comprises the amino acid sequence of SEQ ID NO: 210. In some embodiments, an isolated FN3 domain that binds CD71 comprises the amino acid sequence of SEQ ID NO: 211. In some embodiments, an isolated FN3 domain that binds CD71 comprises the amino acid sequence of SEQ ID NO: 212. In some embodiments, an isolated FN3 domain that binds CD71 comprises the amino acid sequence of SEQ ID NO: 213. In some embodiments, an isolated FN3 domain that binds CD71 comprises the amino acid sequence of SEQ ID NO: 214. In some embodiments, an isolated FN3 domain that binds CD71 comprises the amino acid sequence of SEQ ID NO: 215. In some embodiments, an isolated FN3 domain that binds CD71 comprises the amino acid sequence of SEQ ID NO: 216. In some embodiments, an isolated FN3 domain that binds CD71 comprises the amino acid sequence of SEQ ID NO: 217. In some embodiments, an isolated FN3 domain that binds CD71 comprises the amino acid sequence of SEQ ID NO: 218. In some embodiments, an isolated FN3 domain that binds CD71 comprises the amino acid sequence of SEQ ID NO: 219. In some embodiments, an isolated FN3 domain that binds CD71 comprises the amino acid sequence of SEQ ID NO: 221. In some embodiments, an isolated FN3 domain that binds CD71 comprises the amino acid sequence of SEQ ID NO: 222. In some embodiments, an isolated FN3 domain that binds CD71 comprises the amino acid sequence of SEQ ID NO: 223. In some embodiments, an isolated FN3 domain that binds CD71 comprises the amino acid sequence of SEQ ID NO: 224. In some embodiments, an isolated FN3 domain that binds CD71 comprises the amino acid sequence of SEQ ID NO: 225. In some embodiments, an isolated FN3 domain that binds CD71 comprises the amino acid sequence of SEQ ID NO: 226. In some embodiments, an isolated FN3 domain that binds CD71 comprises the amino acid sequence of SEQ ID NO: 227. In some embodiments, an isolated FN3 domain that binds CD71 comprises the amino acid sequence of SEQ ID NO: 228. In some embodiments, an isolated FN3 domain that binds CD71 comprises the amino acid sequence of SEQ ID NO: 229. In some embodiments, an isolated FN3 domain that binds CD71 comprises the amino acid sequence of SEQ ID NO: 230. In some embodiments, an isolated FN3 domain that binds CD71 comprises the amino acid sequence of SEQ ID NO: 231. In some embodiments, an isolated FN3 domain that binds CD71 comprises the amino acid sequence of SEQ ID NO: 232. In some embodiments, an isolated FN3 domain that binds CD71 comprises the amino acid sequence of SEQ ID NO: 233. In some embodiments, an isolated FN3 domain that binds CD71 comprises the amino acid sequence of SEQ ID NO: 234. In some embodiments, an isolated FN3 domain that binds CD71 comprises the amino acid sequence of SEQ ID NO: 235. In some embodiments, an isolated FN3 domain that binds CD71 comprises the amino acid sequence of SEQ ID NO: 236. In some embodiments, an isolated FN3 domain that binds CD71 comprises the amino acid sequence of SEQ ID NO: 237. In some embodiments, an isolated FN3 domain that binds CD71 comprises the amino acid sequence of SEQ ID NO: 238. In some embodiments, an isolated FN3 domain that binds CD71 comprises the amino acid sequence of SEQ ID NO: 239. In some embodiments, an isolated FN3 domain that binds CD71 comprises the amino acid sequence of SEQ ID NO: 240. In some embodiments, an isolated FN3 domain that binds CD71 comprises the amino acid sequence of SEQ ID NO: 241. In some embodiments, an isolated FN3 domain that binds CD71 comprises the amino acid sequence of SEQ ID NO: 242. In some embodiments, an isolated FN3 domain that binds CD71 comprises the amino acid sequence of SEQ ID NO: 243. In some embodiments, an isolated FN3 domain that binds CD71 comprises the amino acid sequence of SEQ ID NO: 244. In some embodiments, an isolated FN3 domain that binds CD71 comprises the amino acid sequence of SEQ ID NO: 245. In some embodiments, an isolated FN3 domain that binds CD71 comprises the amino acid sequence of SEQ ID NO: 246. In some embodiments, an isolated FN3 domain that binds CD71 comprises the amino acid sequence of SEQ ID NO: 247. In some embodiments, an isolated FN3 domain that binds CD71 comprises the amino acid sequence of SEQ ID NO: 248. In some embodiments, an isolated FN3 domain that binds CD71 comprises the amino acid sequence of SEQ ID NO: 249. In some embodiments, an isolated FN3 domain that binds CD71 comprises the amino acid sequence of SEQ ID NO: 250. In some embodiments, an isolated FN3 domain that binds CD71 comprises the amino acid sequence of SEQ ID NO: 251. In some embodiments, an isolated FN3 domain that binds CD71 comprises the amino acid sequence of SEQ ID NO: 252. In some embodiments, an isolated FN3 domain that binds CD71 comprises the amino acid sequence of SEQ ID NO: 253. In some embodiments, an isolated FN3 domain that binds CD71 comprises the amino acid sequence of SEQ ID NO: 254. In some embodiments, an isolated FN3 domain that binds CD71 comprises the amino acid sequence of SEQ ID NO: 255. In some embodiments, an isolated FN3 domain that binds CD71 comprises the amino acid sequence of SEQ ID NO: 256. In some embodiments, an isolated FN3 domain that binds CD71 comprises the amino acid sequence of SEQ ID NO: 257. In some embodiments, an isolated FN3 domain that binds CD71 comprises the amino acid sequence of SEQ ID NO: 258. In some embodiments, an isolated FN3 domain that binds CD71 comprises the amino acid sequence of SEQ ID NO: 259. In some embodiments, an isolated FN3 domain that binds CD71 comprises the amino acid sequence of SEQ ID NO: 260. In some embodiments, an isolated FN3 domain that binds CD71 comprises the amino acid sequence of SEQ ID NO: 261. In some embodiments, an isolated FN3 domain that binds CD71 comprises the amino acid sequence of SEQ ID NO: 262. In some embodiments, an isolated FN3 domain that binds CD71 comprises the amino acid sequence of SEQ ID NO: 263. In some embodiments, an isolated FN3 domain that binds CD71 comprises the amino acid sequence of SEQ ID NO: 264. In some embodiments, an isolated FN3 domain that binds CD71 comprises the amino acid sequence of SEQ ID NO: 265. In some embodiments, an isolated FN3 domain that binds CD71 comprises the amino acid sequence of SEQ ID NO: 266. In some embodiments, an isolated FN3 domain that binds CD71 comprises the amino acid sequence of SEQ ID NO: 267. In some embodiments, an isolated FN3 domain that binds CD71 comprises the amino acid sequence of SEQ ID NO: 268. In some embodiments, an isolated FN3 domain that binds CD71 comprises the amino acid sequence of SEQ ID NO: 269. In some embodiments, an isolated FN3 domain that binds CD71 comprises the amino acid sequence of SEQ ID NO: 270. In some embodiments, an isolated FN3 domain that binds CD71 comprises the amino acid sequence of SEQ ID NO: 271. In some embodiments, an isolated FN3 domain that binds CD71 comprises the amino acid sequence of SEQ ID NO: 272. In some embodiments, an isolated FN3 domain that binds CD71 comprises the amino acid sequence of SEQ ID NO: 304. In some embodiments, an isolated FN3 domain that binds CD71 comprises the amino acid sequence of SEQ ID NO: 305. In some embodiments, an isolated FN3 domain that binds CD71 comprises the amino acid sequence of SEQ ID NO: 306.

In some embodiments, the FN3 domain binds to human CD71 at site on CD71 that does not compete with transferrin binding to CD71. In some embodiments, the FN3 domain comprises a sequence of SEQ ID NO: 1-7, 10, 12-219, 221-272, 292-299, or 304-306.

In some embodiments, the isolated FN3 domain that binds CD71 comprises an initiator methionine (Met) linked to the N-terminus of the molecule.

Conjugates of the FN3 Domains that Bind CD71 of the Disclosure

In some embodiments, an isolated FN3 domain that binds CD71 conjugated to a heterologous molecule(s) is provided.

In some embodiments, the FN3 domain is conjugated to an oligonucleotide. For example, the oligonucleotide can be used for inhibiting the expression of a gene or mRNA transcript. The oligonucleotide can be a siRNA, miRNA, antisense oligonucleotide, and the like. Accordingly, in some embodiments, the FN3 domain can be conjugated to a polynucleotide, such as, but not limited to, a siRNA molecule, an antisense molecule, a RNA molecule, or a DNA molecule. In some embodiments, FN3 domain that binds CD71 is conjugated to an siRNA molecule using a linker as described herein. In some embodiments, the linker is a chemical linker.

In some embodiments, a composition comprising a polypeptide, such as a polypeptide comprising a FN3 domain, linked to a nucleic acid molecule are provided. The nucleic acid molecule can be, for example, a siRNA molecule.

Accordingly, in some embodiments, the siRNA is a double-stranded RNAi (dsRNA) agent capable of inhibiting the expression of a target gene. The dsRNA agent comprises a sense strand and an antisense strand. In some embodiments, each strand of the dsRNA agent can range from 12-40 nucleotides in length. For example, each strand can be from 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

In some embodiments, the sense strand and antisense strand typically form a duplex dsRNA. The duplex region of a dsRNA agent may be from 12-40 nucleotide pairs in length. For example, the duplex region can be from 14-40 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length.

In some embodiments, the dsRNA comprises one or more overhang regions and/or capping groups of dsRNA agent at the 3′-end, or 5′-end or both ends of a strand. The overhang can be 1-10 nucleotides in length, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be other sequence. The first and second strands can also be joined, e.g., by additional bases to form a hairpin, or by other non-base linkers.

In some embodiments, the nucleotides in the overhang region of the dsRNA agent can each independently be a modified or unmodified nucleotide including, but not limited to 2′-sugar modified, such as, 2-F 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA agent may be phosphorylated. In some embodiments, the overhang region contains two nucleotides having a phosphorothioate between the two nucleotides, where the two nucleotides can be the same or different. In one embodiment, the overhang is present at the 3′-end of the sense strand, antisense strand or both strands. In one embodiment, this 3′-overhang is present in the antisense strand. In one embodiment, this 3′-overhang is present in the sense strand.

The dsRNA agent may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA may also have a blunt end, located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example the single overhang comprises at least two, three, four, five, six, seven, eight, nine, or ten nucleotides in length. In some embodiments, the dsRNA agent may also have two blunt ends, at both ends of the dsRNA duplex.

In some embodiments, every nucleotide in the sense strand and antisense strand of the dsRNA agent may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2 hydroxyl on the ribose sugar; wholesale replacement of the phosphate moiety with “dephospho” linkers; modification or replacement of a naturally occurring base; and replacement or modification of the ribose-phosphate backbone.

In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In one embodiment, each residue of the sense strand and antisense strand is independently modified with 2′-O-methyl or 2′-fluoro.

In some embodiments, at least two different modifications are typically present on the sense strand and antisense strand. Those two modifications may be the 2′-deoxy, 2′-O-methyl or 2′-fluoro modifications, acyclic nucleotides or others.

In one embodiment, the sense strand and antisense strand each comprises two differently modified nucleotides selected from 2′-fluoro, 2′-O-methyl or 2′-deoxy.

The dsRNA agent may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA agent comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. In some embodiments, these terminal three nucleotides may be at the 3′-end of the antisense strand.

In some embodiments, the dsRNA composition is linked by a modified base or nucleoside analogue as described in U.S. Pat. No. 7,427,672, which is incorporated herein by reference.

In some embodiments, the linker can be used to link the FN3 domain as described herein to the sense strand has a formula of I:

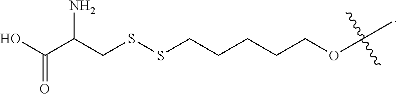 [SMILES: *OCCCCCSSCC(N)C(=O)O]

In some embodiments, the linker can be used to link the FN3 domain as described herein to the antisense strand has a formula of II:

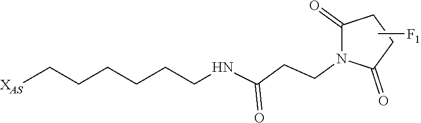

wherein X_{AS} represents the antisense strand and F1 represents a FN3 domain as described herein.

In some embodiments, the linker is covalently attached to F1 through a cysteine residue present on F1, which can be illustrated as follows:

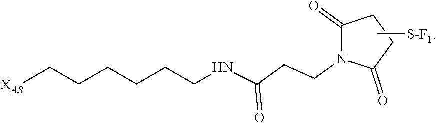

In some embodiments, the linked ds RNA and FN3 domain as described herein has a formula of III:

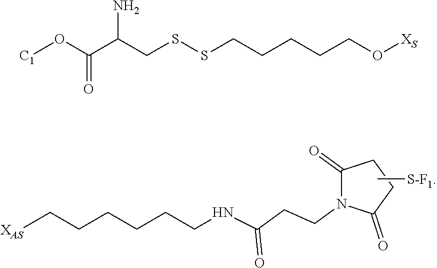

wherein C1 represents the same or different FN3 domain as described herein.

In some embodiments, A1-B1 has a formula of:

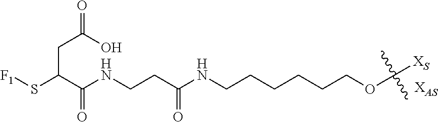

wherein F1 is a polypeptide comprising at least one FN3 domain and is conjugated to L1, L1 is linked to X₅, wherein X₅ is a 5′ to 3′ oligonucleotide sense strand of a double stranded siRNA molecule and X_{AS} is a 3′ to 5′ oligonucleotide antisense strand of a double stranded siRNA molecule; and wherein X₅ and X_{AS} form a double stranded siRNA molecule.

Structures of additional linkers are as follows:

mal-C₂H₄C(O)(NH)—(CH₂)₆— is

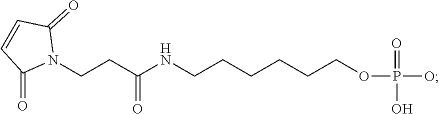

(Mal-(PEG)₁₂)(NH)CH₂)₆) is

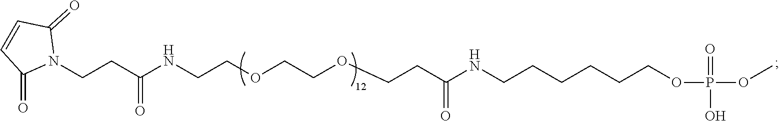

Mal-NH—(CH₂)₆—, which can also be referred to as aminohexyl linker-(CH₂)₆—, is

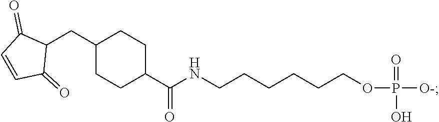

and
Val-Cit Paba, which has the structure:

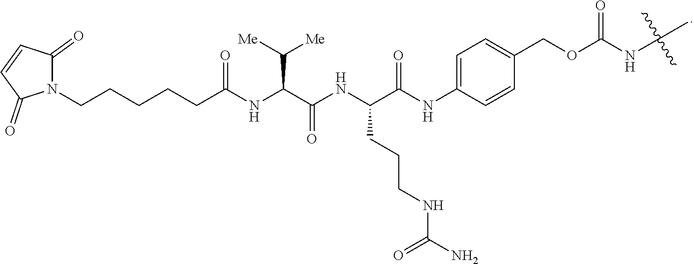 [SMILES: *NC(=O)OCc1ccc(NC(=O)[C@H](CCCNC(N)=O)NC(=O)[C@@H](NC(=O)CCCCCN2C(=O)C=CC2=O)C(C)C)cc1]

As described herein, in some embodiments, the nucleic acid molecules can be modified to include a linker at the 5′ end of the of the sense strand of the dsRNA. In some embodiments, the nucleic acid molecules can be modified to include a vinyl phosphonate at the 5′ end of the of the anti-sense strand of the dsRNA. In some embodiments, the nucleic acid molecules can be modified to include a linker at the 3′ end of the of the sense strand of the dsRNA. In some embodiments, the nucleic acid molecules can be modified to include a vinyl phosphonate at the 3′ end of the of the anti-sense strand of the dsRNA. The linker can be used to link the dsRNA to the FN3 domain. The linker can covalently attach, for example, to a cysteine residue on the FN3 domain that is there naturally or that has been substituted as described herein, and for example, in U.S. Pat. No. 10,196,446, which is hereby incorporated by reference in its entirety.

In some embodiments, the peptide is conjugated to a lipid nanoparticle, which can be used, for example, for cell-specific targeting.

In some embodiments, the protein is conjugated to a binding moiety that targets CD71 or another protein for protein degradation. For example, the protein can be conjugated to a PROTACS (binding moieties for an E3 ubiquitin ligase) and thus deliver the protein to the E3 ligase. These can linked through a linker, such as a glycine-serine linker and the like.

The FN3 domain that binds to CD71 can also be conjugated or linked to another FN3 domain that binds to a different target, other than CD71. This would enable the peptide to be multi-specific (e.g. bi-specific, tri-specific, etc. . . . ), such that it binds to CD71 and another, for example, protein. In some embodiments, the CD71 FN3 binding domain is linked to another FN3 domain that binds to an antigen expressed by a tumor cell (tumor antigen).

In some embodiments, FN3 domains can be linked together by a linker to form a bivalent FN3 domain. The linker can be a flexible linker. In some embodiments, the linker is a G/S linker. In some embodiments the linker has 1, 2, 3, or 4 G/S repeats. A G/S repeat unit is four glycines followed by a serine, e.g. GGGGS (SEQ ID NO: 308). In some embodiments, the FN3 domain comprising two FN3 domains connected by a linker, such as those provided for herein. Exemplary linker include, but are not limited to, (GS)2, (SEQ ID NO: 278), (GGGS)2 (SEQ ID NO: 279), (GGGGS)1-5 (SEQ ID NO: 280), (AP)1-20 (SEQ ID NO: 311); (AP)2 (SEQ ID NO: 281), (AP)5 (SEQ ID NO: 282), (AP)10 (SEQ ID NO: 283), (AP)20 (SEQ ID NO: 284), A(EAAAK)5AAA (SEQ ID NO: 285), or (EAAAK)1-5 (SEQ ID NO: 307). In some embodiments, the linker comprises or is an amino acid sequence of: EAAAKEAAAKEAAAKEAAAK (SEQ ID NO: 300); GGGGSGGGGSGGGGSGGGGS (SEQ ID NO: 301); APAPAPAPAP (SEQ ID NO: 302); or EAAAK (SEQ ID NO: 303).

In some embodiments, the heterologous molecule is a detectable label or a therapeutic agent, such as, but not limited to a cytotoxic agent.

In some embodiments, an FN3 domain that binds CD71 conjugated to a detectable label is provided. Non-limiting examples of detectable labels are provided for herein.

In some embodiments, an FN3 domain that binds CD71 conjugated to a therapeutic agent is provided. Non-limiting examples of therapeutic agents, such as, but not limited to, cytotoxic agents, are provided for herein.

The FN3 domains that bind CD71 conjugated to a detectable label can be used to evaluate expression of CD71 on samples such as tumor tissue in vivo or in vitro. The FN3 domains that bind CD71 conjugated to a detectable label can be used to evaluate expression of CD71 on samples blood, immune cells, or dendritic cells in vivo or in vitro.

Detectable labels include compositions that when conjugated to the FN3 domains that bind CD71 renders CD71 detectable, via spectroscopic, photochemical, biochemical, immunochemical, or other chemical methods.

Exemplary detectable labels include, but are not limited to, radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, cintillants, avidin, streptavidin, protein A, protein G, antibodies or fragments thereof, polyhistidine, Ni2+, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In some embodiments, the detectable label emits a signal as a result of being stimulated by an external stimulus, such as a magnetic or electric, or electromagnetic field.

Exemplary radioactive isotopes may be y-emitting, Auger-emitting, β-emitting, an alpha-emitting or positron-emitting radioactive isotope. Exemplary radioactive isotopes include 3H, 11C, 13C, 15N, 18F, 19F, 55Co, 57Co, 60Co, 61Cu, 62Cu, 64Cu, 67Cu, 68Ga, 72As, 75Br, 86Y, 89Zr, 90Sr, 94mTc, 99mTc, 115 In, 123I, 124I, 125I, 131I, 211At, 212Bi, 213Bi, 223Ra, 226Ra, 225Ac and 227Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e., iodine) to 83 (i.e., bismuth).

In some embodiments, the metal atoms may be atoms suitable for magnetic resonance imaging.

The metal atoms may be metal ions in the form of +1, +2, or +3 oxidation states, such as Ba2+, Bi3+, Cs+, Ca2+, Cr2+, Cr3+, Cr6+, Co2+, Co3+, Cu+, Cu2+, Cu3+, Ga3+, Gd3+, Au+, Au3+, Fe2+, Fe3+, F3+, Pb2+, Mn2+, Mn3+, Mn4+, Mn7+, Hg2+, Ni2+, Ni3+, Ag+, Sr2+, Sn2+, Sn4+, and Zn2+. The metal atoms may comprise a metal oxide, such as iron oxide, manganese oxide, or gadolinium oxide.

Suitable dyes include any commercially available dyes such as, for example, 5 (6)-carboxyfluorescein, IRDye 680RD maleimide or IRDye 800CW, ruthenium polypyridyl dyes, and the like.

Suitable fluorophores are fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The FN3 domains that specifically bind CD71 conjugated to a detectable label may be used, for example, as an imaging agent to evaluate tumor distribution, diagnosis for the presence of tumor cells and/or, recurrence of tumor. The FN3 domains that specifically bind CD71 conjugated to a detectable label may be used, for example, as an imaging agent to evaluate the presence of CD71 positive cells in a variety of tissues in the body, including but not limited to dendritic cells, T-cells, NK cells, B-cells immune cells, muscle cells, and cells of the central nervous system.

In some embodiments, the FN3 domains that specifically bind CD71 are conjugated to a therapeutic agent, such as, but not limited to, a cytotoxic agent.

In some embodiments, the therapeutic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The FN3 domains that bind CD71 conjugated to a therapeutic agent disclosed herein may be used in the targeted delivery of the therapeutic agent to CD71 expressing cells (e.g. tumor cells, dendritic cells, T-cells, NK cells, B-cells immune cells, cells of the central nervous system), and intracellular accumulation therein. Although not bound to any particular theory, this type of delivery can be helpful where systemic administration of these unconjugated agents may result in unacceptable levels of toxicity to normal cells.

In some embodiments, the therapeutic agent can elicit their cytotoxic and/or cytostatic effects by mechanisms such as, but not limited to, tubulin binding, DNA binding, topoisomerase inhibition, DNA cross linking, chelation, spliceosome inhibition, NAMPT inhibition, and HDAC inhibition.

In some embodiments, the therapeutic agent is a spliceosome inhibitor, a NAMPT inhibitor, or a HDAC inhibitor. In some embodiments, the agent is an immune system agonist, for example, TLR7, 8, 9, RIG-I (dsRNA), and STING (CpG) agonists. In some embodiments, the agent is daunomycin, doxorubicin, methotrexate, vindesine, bacterial toxins such as diphtheria toxin, ricin, geldanamycin, maytansinoids or calicheamicin.

In some embodiments, the therapeutic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, or the tricothecenes.

In some embodiments, the therapeutic agent is a radionuclide, such as 212Bi, 131I, 131In, 90Y, or 186Re.

In some embodiments, the therapeutic agent is dolastatin or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45 (12): 3580-3584) and have anticancer and antifungal activity. The dolastatin or auristatin drug moiety may be attached to the FN3 domain through the N (amino) terminus or the C (carboxyl) terminus of the peptidic drug moiety (WO 02/088172), or via any cysteine engineered into the FN3 domain.

In some embodiments, therapeutic agent can be, for example, auristatins, camptothecins, duocarmycins, etoposides, maytansines and maytansinoids, taxanes, benzodiazepines or benzodiazepine containing drugs (e.g., pyrrolo[1,41-benzodiazepines (PBDs), indolinobenzodiazepines, and oxazolidinobenzodiazepines) or vinca alkaloids.

The FN3 domains that specifically bind CD71 may be conjugated to a detectable label using known methods.

In some embodiments, the detectable label is complexed with a chelating agent.

In some embodiments, the detectable label is conjugated to the FN3 domain that binds CD71 via a linker as described above.

The detectable label, therapeutic compound, or the cytotoxic compound may be linked directly, or indirectly, to the FN3 domain that binds CD71 using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis(p-diazoniumbenzoyl)ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiment, the FN3 domain that binds CD71 is removed from the blood via renal clearance.

Isolation of CD71 Binding FN3 Domains from a Library Based on Tencon Sequence

Tencon (SEQ ID NO: 276) is a non-naturally occurring fibronectin type III (FN3) domain designed from a consensus sequence of fifteen FN3 domains from human tenascin-C (Jacobs et al., Protein Engineering, Design, and Selection, 25:107-117, 2012; U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (Bork and Doolittle, Proc Natl Acad Sci USA 89:8990-8992, 1992; U.S. Pat. No. 6,673,901). These loops, or selected residues within each loop, may be randomized in order to construct libraries of fibronectin type III (FN3) domains that may be used to select novel molecules that bind CD71. Table 1 shows positions and sequences of each loop and beta-strand in Tencon (SEQ ID NO: 276).

TABLE 1
Tencon topology

| | Tencon |
|---|---|
| FN3 domain | (SEQ ID NO: 276) |
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Library designed based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

Library designed based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface is described in U.S. Pat. Publ. No. 2013/0226834. Library designed based on Tencon sequence also includes libraries designed based on Tencon variants, such as Tencon variants having substitutions at residues positions 11, 14, 17, 37, 46, 73, or 86 (residue numbering corresponding to SEQ ID NO: 276), and which variants display improve thermal stability. Exemplary Tencon variants are described in US Pat. Publ. No. 2011/0274623, and include Tencon27 (SEQ ID NO: 277) having substitutions E11R, L17A, N46V and E86I when compared to Tencon of SEQ ID NO: 276.

Tencon and other FN3 sequence based libraries may be randomized at chosen residue positions using a random or defined set of amino acids. For example, variants in the library having random substitutions may be generated using NNK codons, which encode all 20 naturally occurring amino acids. In other diversification schemes, DVK codons may be used to encode amino acids Ala, Trp, Tyr, Lys, Thr, Asn, Lys, Ser, Arg, Asp, Glu, Gly, and Cys. Alternatively, NNS codons may be used to give rise to all 20 amino acid residues and simultaneously reducing the frequency of stop codons. Libraries of FN3 domains with biased amino acid distribution at positions to be diversified may be synthesized for example using Slonomics® technology (http://www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well-known IUB code.

The FN3 domains that specifically bind CD71 may be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Natl Acad Sci USA 101, 2806-2810, 2004), and assaying the library for specific binding to PSMA by any method known in the art and described in the Example. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The identified FN3 domains that specifically bind CD71 are further characterized for their binding to CD71, modulation of CD71 activity, internalization, stability, and other desired characteristics.

The FN3 domains that specifically bind CD71 may be generated using any FN3 domain as a template to generate a library and screening the library for molecules specifically binding CD71 using methods provided within. Exemplar FN3 domains that may be used are the 3rd FN3 domain of tenascin C (TN3), Fibcon, and the 10th FN3 domain of fibronectin (FN10). Accordingly, PCT applications WO 2010/051274, WO 2011/137319, and WO 2013/049275 are incorporated herein in their entirety. Standard cloning and expression techniques are used to clone the libraries into a vector or synthesize double stranded cDNA cassettes of the library, to express, or to translate the libraries in vitro. For example ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

In some embodiments, the FN3 domain that binds CD71 is based on Tencon sequence of SEQ ID NO: 276 or Tencon27 sequence of SEQ ID NO: 277, the SEQ ID NO: 276 or the SEQ ID NO: 277, optionally having substitutions at residues positions 11, 14, 17, 37, 46, 73, and/or 86.

In some embodiments, the FN3 protein or polypeptide is one that binds to human CD71 at a site on CD71 that does not compete with transferrin binding to CD71. As used herein, a site on CD71 that does not compete with transferrin binding to CD71 refers to an epitope or part of CD71 where the binding of the FN3 protein does not compete or inhibit the binding of transferrin to CD71. The competition, or lack thereof, can be complete or partial. In some embodiments, the binding also does not inhibit the internalization of transferrin into the cell through its interaction with CD71.

In some embodiments, methods for identifying a FN3 protein that binds to CD71 at a site that does not compete or inhibit transferrin binding to CD71 are provided. In some embodiments, the methods comprise contacting CD71 in the presence of transferrin or an agent that binds to the CD71 transferrin binding site with a test FN3 protein; and identifying a test FN3 protein that binds to CD71 in the presence of transferrin or an agent that binds to the CD71 transferrin binding site. In some embodiments, the method comprises isolating the test FN3 protein that binds to CD71 in the presence of transferrin or an agent that binds to the CD71 transferrin binding site. In some embodiments, the methods comprise sequencing the test FN3 protein that binds to CD71 in the presence of transferrin or an agent that binds to the CD71 transferrin binding site. In some embodiments, the methods comprise preparing or obtaining a nucleic acid sequence encoding the test FN3 protein that binds to CD71 in the presence of transferrin or an agent that binds to the CD71 transferrin binding site. In some embodiments, the methods comprise expressing the test FN3 protein that binds to CD71 in the presence of transferrin or an agent that binds to the CD71 transferrin binding site from a nucleic acid sequence encoding the test FN3 protein that binds to CD71 in the presence of transferrin or an agent that binds to the CD71 transferrin binding site. In some embodiments, the test FN3 protein is expressed in a cell. In some embodiments, the methods comprise isolating and/or purifying the expressed test FN3 protein.

In some embodiments a FN3 protein is provided, wherein the FN3 protein is identified according to any method provided herein.

The FN3 domains that specifically bind CD71 may be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr. Opin. Biotechnol., 12, 371-375, 2001). High thermal stability may increase the yield of the expressed protein, improve solubility or activity, decrease immunogenicity, and minimize the need of a cold chain in manufacturing. Residues that may be substituted to improve thermal stability of Tencon (SEQ ID NO: 276) are residue positions 11, 14, 17, 37, 46, 73, or 86, and are described in US Pat. Publ. No. 2011/0274623. Substitutions corresponding to these residues may be incorporated to the FN3 domain containing molecules disclosed herein.

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“Tm”) temperature, the temperature in ° Celsius (° C.) at which half of the molecules become unfolded, using standard methods. Typically, the higher the Tm, the more stable the molecule. In addition to heat, the chemical environment also changes the ability of the protein to maintain a particular three dimensional structure.

In some embodiments, the FN3 domain that binds CD71 may exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the Tm.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate, lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH₃COOH), halogenated acetic acids), hydrophobic molecules (e.g. phospholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

The FN3 domain that binds CD71 may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include, but are not limited to GS)2, (SEQ ID NO: 278), (GGGS)2 (SEQ ID NO: 279), (GGGGS)1-5 (SEQ ID NO: 280), (AP)1-20 (SEQ ID NO: 311); (AP)2 (SEQ ID NO: 281), (AP)5 (SEQ ID NO: 282), (AP)10 (SEQ ID NO: 283), (AP)20 (SEQ ID NO: 284), A(EAAAK)5AAA (SEQ ID NO: 285), or (EAAAK)1-5 (SEQ ID NO: 307). In some embodiments, the linker is an amino acid sequence of: EAAAKEAAAKEAAAKEAAAK (SEQ ID NO: 300); GGGGGGGGSGGGGSGGGGS (SEQ ID NO: 301); APAPAPAPAP (SEQ ID NO: 302); or EAAAK (SEQ ID NO: 303).

The dimers and multimers may be linked to each other in a N- to C-direction. The use of naturally occurring as well as artificial peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456).

### Half-Life Extending Moieties

The FN3 domains that specifically bind CD71 may incorporate other subunits for example via covalent interaction. In some embodiments, the FN3 domains that specifically bind CD71 further comprise a half-life extending moiety. Exemplary half-life extending moieties are albumin, albumin variants, albumin-binding proteins and/or domains, transferrin and fragments and analogues thereof, and Fc regions. Amino acid sequences of the human Fc regions are well known, and include IgG1, IgG2, IgG3, IgG4, IgM, IgA and IgE Fc regions. In some embodiments, the FN3 domains that specifically bind CD71 may incorporate a second FN3 domain that binds to a molecule that extends the half-life of the entire molecule, such as, but not limited to, any of the half-life extending moieties described herein. In some embodiments, the second FN3 domain binds to albumin, albumin variants, albumin-binding proteins and/or domains, and fragments and analogues thereof.

All or a portion of an antibody constant region may be attached to the FN3 domain that binds CD71 to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and may be further modified by modifying residues in the Fc responsible for these activities (for review; see Strohl, Curr Opin Biotechnol. 20, 685-691, 2009).

Additional moieties may be incorporated into the FN3 domains that specifically bind CD71 such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules disclosed herein.

A pegyl moiety may for example be added to the FN3 domain that binds CD71 by incorporating a cysteine residue to the C-terminus of the molecule, or engineering cysteines into residue positions that face away from the CD71 binding face of the molecule, and attaching a pegyl group to the cysteine using well known methods.

FN3 domains that specifically bind CD71 incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules disclosed herein in in vivo models.

### Polynucleotides, Vectors, Host Cells

In some embodiments, nucleic acids encoding the FN3 domains specifically binding CD71 as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof are provided. Certain exemplary polynucleotides are disclosed herein, however, other polynucleotides which, given the degeneracy of the genetic code or codon preferences in a given expression system, encode the FN3 domains disclosed herein are also within the scope of the disclosure.

In some embodiments, an isolated polynucleotide encodes the FN3 domain specifically binding CD71 comprising the amino acid sequence of SEQ ID Nos: 1-7, 10, 12-219, 221-272, 292-299, or 304-306.

The polynucleotides disclosed herein may be produced by chemical synthesis such as solid phase polynucleotide synthesis on an automated polynucleotide synthesizer and assembled into complete single or double stranded molecules. Alternatively, the polynucleotides disclosed herein may be produced by other techniques such as PCR followed by routine cloning. Techniques for producing or obtaining polynucleotides of a given known sequence are well known in the art.

The polynucleotides disclosed herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

In some embodiments, a vector comprising at least one polynucleotide disclosed herein is provided. Such vectors may be plasmid vectors, viral vectors, vectors for baculovirus expression, transposon based vectors or any other vector suitable for introduction of the polynucleotides disclosed herein into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In some embodiments, a host cell comprising the vector is provided. The FN3 domain that specifically bind CD71 may be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, Antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

In some embodiments, a method of producing the isolated FN3 domain that binds CD71, comprising culturing the isolated host cell under conditions such that the isolated FN3 domain that binds CD71 is expressed, and purifying the FN3 domain.

The FN3 domains that bind CD71 may be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography, or high performance liquid chromatography (HPLC).

In some embodiments, the FN3 domain specifically binding CD71 comprises the amino acid sequence of SEQ ID Nos: 1-7, 10, 12-219, 221-272, 292-299, or 304-306, wherein a histidine tag has been appended to the N-terminal or C-terminal end of the polypeptide for ease of purification. In some embodiments, the histidine tag (His-tag) comprises six histidine residues (SEQ ID NO: 309). In further embodiments, the His-tag to connected to the FN3 domain by at least one glycine residue or about 2 to about 4 glycine residues. Accordingly, after purification of the FN3 domain and cleavage of the His-tag from the polypeptide one or more glycine may be left on the N-terminus or C-terminus. In some embodiments, if the His-tag is removed from the N-terminus all of the glycines are removed. In some embodiments, if the His-tag is removed from the C-terminus one or more of the glycines are retained.

In some embodiments, the FN3 domain specifically binding CD71 comprises the amino acid sequence of SEQ ID Nos: 1-7, 10, 12-219, 221-272, 292-299, or 304-306, wherein the N-terminal methionine is retained after purification of the FN3 domain.

### Kits

In some embodiments, a kit comprising the FN3 domain that binds CD71 is provided.

The kit may be used for therapeutic uses and as a diagnostic kit.

In some embodiments, the kit comprises the FN3 domain that binds CD71 and reagents for detecting the FN3 domain. In some embodiments, the kit comprises a bivalent FN3 domain. The kit can include one or more other elements including: instructions for use; other reagents, e.g., a label, an agent useful for chelating, or otherwise coupling, a radioprotective composition; devices or other materials for preparing the FN3 domain that binds CD71 for administration for imaging, diagnostic or therapeutic purpose; pharmaceutically acceptable carriers; and devices or other materials for administration to a subject.

In some embodiments, the kit comprises the FN3 domain that binds CD71 comprising the amino acid sequences of one of SEQ ID Nos: 1-7, 10, 12-219, 221-272, 292-299, or 304-306.

### Uses of CD71 Binding FN3 Domains

The FN3 domains that specifically bind CD71 or conjugates thereof may be used to diagnose, monitor, modulate, treat, alleviate, help prevent the incidence of, or reduce the symptoms of human disease or specific pathologies in cells, tissues, organs, fluid, or, generally, a host.

In some embodiments, the FN3 domain can facilitate delivery into CD71 positive tissues (eg. Skeletal muscle, smooth muscle) for treatment of muscle diseases.

In some embodiments, the FN3 domain can facilitate delivery to activated lymphocytes, dendritic cells, T-cells, NK cells and B-cells, or other immune cells for treatment of immunological diseases.

In some embodiments, the FN3 domains that specifically bind CD71 or conjugates thereof may also be used in imaging CD71 positive tumor tissue in a subject. The methods disclosed herein may be used with an animal patient belonging to any classification. Examples of such animals include mammals such as humans, rodents, dogs, cats and farm animals.

In some embodiments, a method of diagnosing a subject having, or who is likely to develop cancer of a tissue based on the expression of CD71 by cells of the cancer tissue, methods of predicting success of immunotherapy, methods of prognosis, and methods of treatment are provided.

In some embodiments, a method of detecting CD71-expressing cancer cells in a tumor tissue is provided, the method comprising: obtaining a sample of the tumor tissue from a subject; detecting whether CD71 is expressed in the tumor tissue by contacting toe sample of the tumor tissues with the FN3 domain that binds CD71 comprising the amino acid sequence of one of SEQ ID Nos: 1-7, 10, 12-219, 221-272, 292-299, or 304-306, and detecting the binding between CD71 and the FN3 domain. In some embodiments, methods of treating cancer in a subject in need thereof are provided. In some embodiments, the method comprises administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, the CD71 cell is a cell involved in a CNS diseases, inflammatory/immune diseases, such as MS & infectious diseases of the brain. In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis.

In some embodiments, the polypeptide that binds to CD71 is directed to muscle cells. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, methods of treating Pompe disease (GSD2, acid alpha-glucosidase (GAA) deficiency) in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent.

In some embodiments, methods of treating glycogen storage disease in a subject in need thereof, the method comprising administering a composition provided herein are provided. In some embodiments, the glycogen storage disease is selected from the group consisting of Cori's disease or Forbes' disease (GSD3, Glycogen debranching enzyme (AGL) deficiency), McArdle disease (GSD5, Muscle glycogen phosphorylase (PYGM) deficiency), type II Diabetes/diabetic nephropathy, Aldolase A Deficiency GSD12, Lafora Disease, hypoxia, Andersen disease (GSD4, Glycogen debranching enzyme (GBE1) deficiency), Tarui's Disease (GSD7, Muscle phosphofructokinase (PFKM) deficiency), and adult polyglucosan body disease. In some embodiments, the glycogen storage disease is selected from the group consisting of Glycogen synthase (GYS2) deficiency (GSD0), Glucose-6-phosphatase (G6PC/SLC37A4) deficiency (GSD1, von Gierke's disease), Hers' disease (GSD6, Liver glycogen phosphorylase (PYGL) or Muscle phosphoglycerate mutase (PGAM2) deficiency), Phosphorylase kinase (PHKA2/PHKB/PHKG2/PHKA1) deficiency (GSD9), Phosphoglycerate mutase (PGAM2) deficiency (GSD10), Muscle lactate dehydrogenase (LDHA) deficiency (GSD11), Fanconi-Bickel syndrome (GSD 11, Glucose transporter (GLUT2) deficiency, Aldolase A deficiency (GSD 12), β-enolase (ENO3) deficiency (GSD13), and Glycogenin-1 (GYG1) deficiency (GSD15).

In some embodiments, the polypeptide that binds to CD71 is directed to immune cells. In some embodiments, the polypeptide that binds to CD71 is directed to dendritic cells, T-cells, NK cells, or B-cells. In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, and immune thrombocytopeniaurpura.

In some embodiments, the tissue can be tissue of any organ or anatomical system, that expresses CD71.

In some embodiments, CD71 expression may be evaluated using known methods, such as immunohistochemistry or ELISA.

In some embodiments, a method of isolating CD71 expressing cells is provided, the method comprising: obtaining a sample from a subject; contacting the sample with the FN3 domain that binds CD71 comprising the amino acid sequence of one of SEQ ID Nos: 1-7, 10, 12-219, 221-272, 292-299, or 304-306, and isolating the cells bound to the FN3 domains.

In some embodiments, a method of detecting CD71-expressing cancer cells in a tumor tissue is provided, the method comprising: conjugating the FN3 domain that binds CD71 comprising the amino acid sequence of one of SEQ ID Nos: 1-7, 10, 12-219, 221-272, 292-299, or 304-306 to a detectable label to form a conjugate; administering the conjugate to a subject; and visualizing the CD71 expressing cancer cells to which the conjugate is bound.

In some embodiments, methods of treating cancer in a subject in need thereof are provided. In some embodiments, the method comprises administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, a method of treating a subject having cancer is provided, the method comprising administering to the subject a FN3 domain that binds CD71. In some embodiments, the FN3 domain is conjugated to a therapeutic agent (e.g. cytotoxic agent, an oligonucleotide, such as a siRNA, antisense, and the like, a FN3 domain that binds to another target, and the like).

In some embodiments, the subject has a solid tumor.

In some embodiments, the solid tumor is a melanoma.

In some embodiments, the solid tumor is a lung cancer. In some embodiments, the solid tumor is a non-small cell lung cancer (NSCLC). In some embodiments, the solid tumor is a squamous non-small cell lung cancer (NSCLC). In some embodiments, the solid tumor is a non-squamous NSCLC. In some embodiments, the solid tumor is a lung adenocarcinoma.

In some embodiments, the solid tumor is a renal cell carcinoma (RCC).

In some embodiments, the solid tumor is a mesothelioma.

In some embodiments, the solid tumor is a nasopharyngeal carcinoma (NPC).

In some embodiments, the solid tumor is a colorectal cancer.

In some embodiments, the solid tumor is a prostate cancer. In some embodiments, the solid tumor is castration-resistant prostate cancer.

In some embodiments, the solid tumor is a stomach cancer.

In some embodiments, the solid tumor is an ovarian cancer.

In some embodiments, the solid tumor is a gastric cancer.

In some embodiments, the solid tumor is a liver cancer.

In some embodiments, the solid tumor is pancreatic cancer.

In some embodiments, the solid tumor is a thyroid cancer.

In some embodiments, the solid tumor is a squamous cell carcinoma of the head and neck.

In some embodiments, the solid tumor is a carcinomas of the esophagus or gastrointestinal tract.

In some embodiments, the solid tumor is a breast cancer.

In some embodiments, the solid tumor is a fallopian tube cancer.

In some embodiments, the solid tumor is a brain cancer.

In some embodiments, the solid tumor is an urethral cancer.

In some embodiments, the solid tumor is a genitourinary cancer.

In some embodiments, the solid tumor is an endometriosis.

In some embodiments, the solid tumor is a cervical cancer.

In some embodiments, the solid tumor is a metastatic lesion of the cancer.

In some embodiments, the subject has a hematological malignancy. In some embodiments, the hematological malignancy is a lymphoma, a myeloma or a leukemia. In some embodiments, the hematological malignancy is a B cell lymphoma. In some embodiments, the hematological malignancy is Burkitt's lymphoma. In some embodiments, the hematological malignancy is Hodgkin's lymphoma. In some embodiments, the hematological malignancy is a non-Hodgkin's lymphoma.

In some embodiments, the hematological malignancy is a myelodysplastic syndrome.

In some embodiments, the hematological malignancy is an acute myeloid leukemia (AML). In some embodiments, the hematological malignancy is a chronic myeloid leukemia (CML). In some embodiments, the hematological malignancy is a chronic myelomoncytic leukemia (CMML).

In some embodiments, the hematological malignancy is a multiple myeloma (MM).

In some embodiments, the hematological malignancy is a plasmacytoma.

In some embodiments, the compositions or pharmaceutical compositions provided herein may be administered alone or in combination with other therapeutics, that is, simultaneously or sequentially. In some embodiments, the other or additional therapeutics are other anti-tumor agent or therapeutics. Different tumor types and stages of tumors can require the use of various auxiliary compounds useful for treatment of cancer. For example, the compositions provided herein can be used in combination with various chemotherapeutics such as taxol, tyrosine kinase inhibitors, leucovorin, fluorouracil, irinotecan, phosphatase inhibitors, MEK inhibitors, among others. The composition may also be used in combination with drugs which modulate the immune response to the tumor such as anti-PD-1 or anti-CTLA-4, among others. Additional treatments can be agents that modulate the immune system, such antibodies that target PD-1 or PD-L1.

In some embodiments, the polypeptide that binds to CD71 is directed to the central nervous system. In some embodiments, methods of treating a neurological condition and/or a brain tumor in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the brain tumor is selected from the group consisting of nonmalignant, benign, and malignant brain tumors. In some embodiments, the neurological condition is selected from the group consisting of Alzheimer's Disease, Amyotrophic Lateral Sclerosis, Parkinson's Disease, Lafora Disease, Pompe Disease, adult polyglucosan body disease, stroke, spinal cord injury, ataxia, Bell's Palsy, cerebral aneurysm, epilepsy, seizures, Guillain-Barre Syndrome, multiple sclerosis, muscular dystrophy, neurocutaneous syndromes, migraine, encephalitis, septicemia, and myasthenia gravis. In some embodiments, a method of treating a neurological condition and/or a brain tumor in a subject, the method comprising administering to the subject a FN3 domain that binds CD71 and the FN3 domain is conjugated to a therapeutic agent (e.g. cytotoxic agent, an oligonucleotide, such as a siRNA, antisense, and the like, a FN3 domain that binds to another target, and the like).

In some embodiments, methods of treating Pompe disease (GSD2, acid alpha-glucosidase (GAA) deficiency) in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, a method of treating a Pompe disease (GSD2, acid alpha-glucosidase (GAA) deficiency) in a subject, the method comprising administering to the subject a FN3 domain that binds CD71 and the FN3 domain is conjugated to a therapeutic agent (e.g. cytotoxic agent, an oligonucleotide, such as a siRNA, antisense, and the like, a FN3 domain that binds to another target, and the like).

In some embodiments, the polypeptide that binds to CD71 is directed to immune cells. In some embodiments, the polypeptide that binds to CD71 is directed to dendritic cells. In some embodiments, methods of treating an autoimmune disease in a subject in need thereof are provided. In some embodiments, the methods comprise administering to the subject a polypeptide or the pharmaceutical composition that binds to CD71. In some embodiments, that the polypeptide is a FN3 domain that binds to CD71. In some embodiments, the polypeptide comprises a sequence such as SEQ ID Nos: 1-7, 10, 12-219, 221-272, 292-299, or 304-306, or a polypeptide as provided herein that is linked to or conjugated to a therapeutic agent. In some embodiments, the autoimmune disease is selected from the group consisting of rheumatoid arthritis, Hashimoto's autoimmune thyroiditis, celiac disease, diabetes mellitus type 1, vitiligo, rheumatic fever, pernicious anemia/atrophic gastritis, alopecia areata, and immune thrombocytopeni purpura. In some embodiments, a method of treating an autoimmune disease in a subject, the method comprising administering to the subject a FN3 domain that binds CD71 and the FN3 domain is conjugated to a therapeutic agent (e.g. cytotoxic agent, an oligonucleotide, such as a siRNA, antisense, and the like, a FN3 domain that binds to another target, and the like).

In some embodiments, the FN3 domains that specifically bind CD71 or conjugates thereof that may be used to diagnose, monitor, modulate, treat, alleviate, help prevent the incidence of, or reduce the symptoms of human disease or specific pathologies in cells, tissues, organs, fluid, or, generally, a host, also exhibit the property of being able to cross the blood brain barrier. The blood-brain barrier (BBB) prevents most macromolecules (e.g., DNA, RNA, and polypeptides) and many small molecules from entering the brain. The BBB is principally composed of specialized endothelial cells with highly restrictive tight junctions, consequently, passage of substances, small and large, from the blood into the central nervous system is controlled by the BBB. This structure makes treatment and management of patients with neurological diseases and disorders (e.g., brain cancer) difficult as many therapeutic agents cannot be delivered across the BBB with desirable efficiency. Additional conditions that involve disruptions of the BBB include: stroke, diabetes, seizures, hypertensive encephalopathy, acquired immunodeficiency syndrome, traumatic brain injuries, multiple sclerosis, Parkinson's disease (PD) and Alzheimer disease. This ability is especially useful for treating brain cancers including for example: astrocytoma, medulloblastoma, glioma, ependymoma, germinoma (pinealoma), glioblastoma multiform, oligodendroglioma, schwannoma, retinoblastoma, and congenital tumors; or a cancer of the spinal cord, e.g., neurofibroma, meningioma, glioma, and sarcoma. In certain embodiments, the FN3 domains that specifically bind CD71 comprising the amino acid sequence of one of SEQ ID Nos: 1-7, 10, 12-219, 221-272, 292-299, or 304-306 or conjugates thereof, are useful to deliver a therapeutic or cytotoxic agent, for example, across the blood brain barrier.

In some embodiments, the polypeptide that can facilitates the transport of a therapeutic across the BBB is a protein comprising a sequence of SEQ ID NO: 1-7, 10, 12-219, 221-272, 292-299, or 304-306.

“Treat” or “treatment” refers to the therapeutic treatment and prophylactic measures, wherein the object is to prevent or slow down (lessen) an undesired physiological change or disorder, such as the development or spread of cancer. In some embodiments, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the FN3 domains that specifically bind CD71 may vary according to factors such as the disease state, age, sex, and weight of the individual. Exemplary indicators of an effective FN3 domain that binds CD71 is improved well-being of the patient, decrease or shrinkage of the size of a tumor, arrested or slowed growth of a tumor, and/or absence of metastasis of cancer cells to other locations in the body.

### Administration/Pharmaceutical Compositions

In some embodiments, pharmaceutical compositions of the FN3 domains that specifically bind CD71, optionally conjugated to a detectable label, therapeutic, or a cytotoxic agent disclosed herein and a pharmaceutically acceptable carrier, are provided. For therapeutic use, the FN3 domains that specifically bind CD71 may be prepared as pharmaceutical compositions containing an effective amount of the domain or molecule as an active ingredient in a pharmaceutically acceptable carrier. “Carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules disclosed herein in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the FN3 domains disclosed herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intra-articular, intrabronchial, intra-abdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardial, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### EXAMPLES

The following examples are illustrative of the embodiments disclosed herein. These examples are provided for the purpose of illustration only and the embodiments should in no way be construed as being limited to these examples, but rather should be construed to encompass any and all variations which become evidence as a result of the teaching provided herein. Those of skill in the art will readily recognize a variety of non-critical parameters that could be changed or modified to yield essentially similar results.

### Example 1. Construction of Tencon Libraries with Randomized Loops

Tencon (SEQ ID NO: 276) is an immunoglobulin-like scaffold, fibronectin type III (FN3) domain, designed from a consensus sequence of fifteen FN3 domains from human tenascin-C (Jacobs et al., Protein Engineering, Design, and Selection, 25:107-117, 2012; U.S. Pat. No. 8,278,419). The crystal structure of Tencon shows six surface-exposed loops that connect seven beta-strands. These loops, or selected residues within each loop, can be randomized in order to construct libraries of fibronectin type III (FN3) domains that can be used to select novel molecules that bind to specific targets. Various libraries were generated using the Tencon scaffold and various design strategies. In general, libraries TCL1 and TCL2 produced good binders. Generation of TCL1 and TCL2 libraries are described in detail in Int. Pat. Publ. No. WO/2014081944A2.

### Example 2: Generation of Tencon Libraries Having Alternative Binding Surfaces

The choice of residues to be randomized in a particular library design governs the overall shape of the interaction surface created. X-ray crystallographic analysis of an FN3 domain containing scaffold protein selected to bind maltose binding protein (MBP) from a library in which the BC, DE, and FG loops were randomized was shown to have a largely curved interface that fits into the active site of MBP (Koide et al., Proc. Natl. Acad. Sci. USA 104:6632-6637, 2007). In contrast, an ankyrin repeat scaffold protein that was selected to bind to MBP was found to have a much more planar interaction surface and to bind to the outer surface of MBP distant from the active (Binz et al., Nat. Biotechnol. 22:575-582, 2004). These results suggest that the shape of the binding surface of a scaffold molecule (curved vs. flat) may dictate what target proteins or specific epitopes on those target proteins are able to be bound effectively by the scaffold. Published efforts around engineering protein scaffolds containing FN3 domains for protein binding has relied on engineering adjacent loops for target binding, thus producing curved binding surfaces. This approach may limit the number of targets and epitopes accessible by such scaffolds.

Tencon and other FN3 domains contain two sets of CDR-like loops lying on the opposite faces of the molecule, the first set formed by the BC, DE, and FG loops, and the second set formed by the AB, CD, and EF loops. The two sets of loops are separated by the beta-strands that form the center of the FN3 structure. If the image of the Tencon is rotated by 90 degrees, an alternative surface can be visualized. This slightly concave surface is formed by the CD and FG loops and two antiparallel beta-strands, the C and the F beta-strands, and is herein called the C-CD-F-FG surface. The C-CD-F-FG surface can be used as a template to design libraries of protein scaffold interaction surfaces by randomizing a subset of residues that form the surface. Beta-strands have a repeating structure with the side chain of every other residue exposed to the surface of the protein. Thus, a library can be made by randomizing some or all surface exposed residues in the beta strands. By choosing the appropriate residues in the beta-strands, the inherent stability of the Tencon scaffold should be minimally compromised while providing a unique scaffold surface for interaction with other proteins.

A full description of the methods used to construct this library is described in US. Pat. Publ. No. 2013/0226834.

The two beta strands forming the C-CD-F-FG surface in Tencon27 have a total of 9 surface exposed residues that could be randomized; C-strand: S30, L32, Q34, Q36; F-strand: E66, T68, S70, Y72, and V74, while the CD loop has 6 potential residues: S38, E39, K40, V41, G42, and E43 and the FG loop has 7 potential residues: K75, G76, G77, H78, R79, S80, and N81. Select residues were chosen for inclusion in the TCL14 design due to the larger theoretical size of the library if all 22 residues were randomized.

Thirteen positions in Tencon were chosen for randomizing: L32, Q34 and Q36 in C-strand, S38, E39, K40 and V41 in CD-loop, T68, S70 and Y72 in F-strand, H78, R79, and N81 in FG-loop. In the C and F strands S30 and E66 were not randomized as they lie just beyond the CD and FG loops and do not appear to be as apparently a part of the C-CD-F-FG surface. For the CD loop, G42 and E43 were not randomized as glycine, providing flexibility, can be valuable in loop regions, and E43 lies at the junction of the surface. The FG loop had K75, G76, G77, and S80 excluded. The glycines were excluded for the reasons above while careful inspection of the crystal structures revealed S80 making key contacts with the core to help form the stable FG loop. K75 faces away from the surface of the C-CD-F-FG surface and was a less appealing candidate for randomization. Although the above mentioned residues were not randomized in the original TCL14 design, they could be included in subsequent library designs to provide additional diversity for de novo selection or for example for an affinity maturation library on a select TCL14 target specific hit.

Subsequent to the production of TCL14, 3 additional Tencon libraries of similar design were produced. These two libraries, TCL19, TCL21 and TCL23, are randomized at the same positions as TCL14 (see above) however the distribution of amino acids occurring at these positions is altered. TCL19 and TCL21 were designed to include an equal distribution of 18 natural amino acids at every position (5.55% of each), excluding only cysteine and methionine. TCL23 was designed such that each randomized position approximates the amino acid distribution found in the HCDR3 loops of functional antibodies (Birtalan et al., J. Mol. Biol. 377:1518-1528, 2008). As with the TCL21 library, cysteine and methionine were excluded.

A third additional library was built to expand potential target binding surface of the other libraries library. In this library, TCL24, 4 additional Tencon positions were randomized as compared to libraries TCL14, TCL19, TCL21, and TCL23. These positions include N46 and T48 from the D strand and S84 and 186 from the G strand. Positions 46, 48, 84, and 86 were chosen in particular as the side chains of these residues are surface exposed from beta-strands D and G and lie structurally adjacent to the randomized portions of the C and F strand, thus increasing the surface area accessible for binding to target proteins. The amino acid distribution used at each position for TCL24 is identical to that described for TCL19 and TCL21.

### Generation of TCL21, TCL23, and TCL24 Libraries

The TCL21 library was generated using Colibra library technology (Isogenica) in order to control amino acid distributions. TCL19, TCL23, and TCL24 gene fragments were generated using Slonomics technology (Morphosys) to control amino acid distributions. PCR was used to amplify each library following initial synthesis followed by ligation to the gene for RepA in order to be used in selections using the CIS-display system (Odegrip et al., Proc. Natl. Acad. Sci. USA 101:2806-2810, 2004) as described above for the loop libraries.

### Example 3: Selection of Fibronectin Type III (FN3) Domains that Bind CD71

### Panning and Biochemical Screening

FN3 domains specific for human CD71 were selected via CIS-Display (Odegrip et al 2004) using recombinant biotinylated CD71 extracellular domain (Sino Biologics) with an N-terminal 6His tag (SEQ ID NO: 309). For in vitro transcription and translation (ITT), 3 μg of DNA from FN3 domain libraries TCL18, TCL19, TCL21, TCL23, and TCL24 were used, with unbound library members removed by washing. DNA was eluted from the target protein by heating and amplified by PCR using KOD polymerase for further rounds of panning. High affinity binders were isolated by successively lowering the concentration of target CD71 during each round from 400 nM to 100 nM and increasing the washing stringency. Outputs from the fifth round panning were subjected to four additional rounds of off-rate selection. The biotinylated target antigen concentration was reduced from 25 nM in rounds 6 and 7 to 2.5 nM in rounds 8 and 9.

Following panning, genes encoding the selected FN3 domains were amplified by PCR, subcloned into a pET vector modified to include a ligase independent cloning site, and transformed into BL21 (DE3) (Stratagene) cells for soluble expression in E. coli using standard molecular biology techniques. A gene sequence encoding a C-terminal poly-histidine tag was added to each FN3 domain to enable purification and detection.

To screen for FN3 domains that specifically bind CD71, streptavidin-coated Maxisorp plates (Nunc catalog 436110) were blocked for 1 hour in Starting Block T20 (Pierce) and then coated with biotinylated CD71 (using same antigen as in panning) or negative controls (an unrelated Fc-fused recombinant protein and human serum albumin) for 1 hour. Plates were rinsed with TBST and diluted lysate was applied to plates for 1 hour. Following additional rinses, wells were treated with HRP-conjugated anti-V5 tag antibody (Abcam, ab1325), for 1 hour and then assayed with POD Roche, 11582950001). The DNA from FN3 domain lysates with signals at least 10-fold ELISA signal above that of streptavidin controls were sequenced resulting in 23 unique, readable FN3 domain sequences isolated from Round 9 screening.

### Size Exclusion Chromatography Analysis

Size exclusion chromatography was used to determine the aggregation state of anti-CD71 FN3 domains. Aliquots (10 μL) of each purified FN3 domain were injected onto a Superdex 75 5/150 column (GE Healthcare) at a flow rate of 0.3 mL/min in a mobile phase of PBS pH 7.4. Elution from the column was monitored by absorbance at 280 nm. Tencon protein was included in each run as a control. Agilent ChemStation software was used to analyze the elution profiles.

### High-Throughput Expression and Conjugation

Clones identified were grown in duplicate 5 mL cultures in 24 well deep block plates. Briefly, 5 mL/well of TB media supplemented with 50 μg/mL Kanamycin was seeded with 150 μL of overnight culture and grown for about 3 hours at 37° C. with shaking at 220 rpm (OD600˜1). Cultures were induced with IPTG to a final concentration of 1 mM for an additional 4 hours at 37° C., 220 rpm. Bacterial pellets were recovered by centrifugation at 2250×g for 15 minutes. 600 μL/well BugBuster HT (Novagen) supplemented with lysozyme (Sigma) at 0.2 mg/mL was added to each well; pellets were dissociated by pipette and then shaken vigorously on a platform shake for about 30 minutes until pellets were lysed. Plates were spun at 2250×g for 15 minutes to clarify lysates and the 2 600-μL aliquots for each sample were combined. His-tagged FN3 domains were purified on His Trap plates (GE) according to the manufacturer's instructions followed by buffer exchange into TBS using Zeba Spin 7K desalt plates (Thermo Scientific). Protein concentrations were assessed by Nanodrop. For conjugation to GlyGly-VC-MMAF (SEQ ID NO: 310), FN3 domain (30 μM) was mixed with 150 μM GlyGlyVC-MMAF (SEQ ID NO: 310) (Concortis) and 1 μM Sortase A in a total volume of 200 μL. Conjugations were allowed to proceed for 1.5 hours at room temperature and purified again using a 96 well His Multitrap HP plate from GE Healthcare according to the manufacturer's instructions. Buffer exchange into PBS was achieved using Zeba desalt plates followed by sterile filtering using Multiscreen HTS GV plates (Durapore) with centrifugation at 3000×g for 2 mins. Concentrations were assessed by Nanodrop.

### CD71 Mediated SK-BR3 Cell Killing Assay.

Cell killing was assessed by measuring viability of the CD71-overexpressing human tumor cell line H1573 w/SKBR3 following exposure to the cysteine variant-cytotoxin conjugates. Cells were plated in black-well, clear bottomed, tissue culture-treated plates (Falcon 353219) at 7000/well in 100 μL/well of phenol red free RPMI media (Gibco11835-030) with 5% fetal bovine serum (Gibco). Cells were allowed to attach overnight at 37° C. in a humidified 5% CO2 atmosphere. Medium was aspirated from 96-well plate and cells were treated with 50 μL of fresh media and 50 μL of 2× inhibitor made up in fresh media. Cell viability was determined by an endpoint assay with Cell TiterGlo (Promega) at 70 hours. IC50 values were determined by fitting data to the equation for a sigmoidal dose response with variable slope using GraphPad Prism 5 (GraphPad Software).

### Binding of Selected Clones by Dose-Response ELISA

Selected clones are analyzed by ELISA to determine EC50 values for binding. Briefly, Maxisorb plates are coated with streptavidin at 5 μg/ml overnight at 4 C. Plates were then blocked with StartingBlock (ThermoFisher) at room temperature for 1 hour and then washed with TBS-Tween. Biotinylated CD71 (2 μg/ml) was captured onto the streptavidin plates and serially diluted FN3 proteins were added to appropriate wells for 1 hour at room temperature. After washing, bound FN3 proteins was detected with anti-V5 tag antibody, which is conjugated to HRP and POD substrate and a luminescence plate reader. Luminescence values are plotted as a function of concentration and fit to a dose response using PRISM to determine EC50 values for binding.

Identification of internalizing FN3 domains via toxin conjugates. The FN3 domains were conjugated to the cytotoxic tubulin inhibitor momomethyl auristatin F (MMAF) via an enzyme-cleavable Val-Cit linker or a non-cleavable PEG4 linker (VC-MMAF) using the methodology described for the NEM conjugation. Cell killing was assessed by measuring viability of the SKBR-3 cells following exposure to the cysteine variant-cytotoxin conjugates. Cells are plated in white-well, opaque bottomed, tissue culture-treated plates (Fisher, PI15042) at 3000/well in 50 μL/well of phenol red RPMI media (Gibco, 11875093) with 10% fetal bovine serum (Gibco). Cells are allowed to attach overnight at 37° C. in a humidified 5% CO2 atmosphere. Cells are treated with 25 μL of fresh media and 25 μL of 4× inhibitor made up in fresh media. Cell viability is determined by an endpoint assay with Cell TiterGlo (Promega) at 72 hours. IC50 values are determined by fitting data to the equation for a sigmoidal dose response with variable slope using GraphPad Prism (GraphPad Software).

### Bivalent FN3 Protein

A bivalent FN3 protein is produced using two FN3 domains connected by a 4 repeat G/S linker or other appropriate polypeptide linker. The bivalent FN3 protein is conjugated to VC-MMAF as described and assessed for cytotoxicity in SK-BR3 cells. The IC50 value for bivalent molecule is often found to be better than the monovalent version.

### Competition for Transferrin Binding and Internalization

FN3 domain vcMMAF conjugates were screened for competition with human transferrin using the cytotoxicity assay described above. FN3 domains were screened in the absence or presence of 0.6 uM holo-human transferrin (T0665-100 MG).

pHrodo-Tf Assay

CD71-targeting Centyrins were evaluated for their ability to compete with transferrin for binding to the transferrin receptor. Cells are treated with transferrin that is directly conjugated to pHrodo-Red, a dye that fluoresces in acidic compartments and is therefore visible upon cellular uptake into endosomal and lysosomal compartments. Imaging of pHrodo-transferrin (pHrodo-Tf) is performed on an Incucyte, allowing real-time measurement of Tf uptake. When cells are incubated with pHrodo-Tf and a molecule that competes with Tf for CD71 binding, the pHrodo signal is reduced or eliminated. Centyrins that do not compete with Tf for CD71 binding have no impact on the pHrodo signal.

### Example 4: Selection of Fibronectin Type III (FN3) Domains that Bind CD71 and are not Competitive with Transferrin

To identify CD71 binding FN3 domains that were either not competitive or minimally competitive with transferrin a biased CIS-display strategy was designed. In short, using the output recovered after 5 rounds of panning on the ECD of human CD71 (Example 3), additional rounds of off-rate selection were performed as described in Example 3 with the addition of either 1) a wash step with human holo transferrin to elute FN3 proteins that bound at the same site as transferrin before the final elution step or 2) elution of FN3 domain binders with monoclonal antibody OKT9. FN3 domains recovered from the transferrin wash strategy and the OKT9 elution strategy were PCR amplified and cloned into pET vector as previously described (Example 3). 228 FN3 domains that specifically bound huCD71 were confirmed by solution ELISA for binding to huCD71 ECD. A subset of the unique binders was analyzed by SEC, conjugated to MMAF and assessed for internalization via cell viability assay in SKBR-3 cells+/−holo human transferrin. The polypeptides were found to be internalized by the receptor. Integral Molecular performed Membrane Proteome Array (MPA) assay to profile the specificity of ABX1198 (SEQ ID NO: 209), ABX1142 (SEQ ID NO: 209 plus a His-tag) and ABX1100 (SEQ ID NO: 209 plus siRNA pair with linker) against the library of human membrane proteins. The MPA library contains over 6000 human membrane proteins, including 94% of all single-pass, multi-pass and GPI anchored proteins including GPCRs, ion channels and transporters with each membrane protein uniquely expressed in an avian QT6 cell background. Flow cytometry is used to directly detect ligand (FN3 domain) binding to membrane proteins individually expressed in unfixed cells.

ABX1198 (SEQ ID NO: 209), ABX1142 (SEQ ID NO: 209 plus a His-tag) and ABX1100 (SEQ ID NO: 209 plus siRNA pair with linker) were screened at the concentration with optimal signal/background noise ratio, 1.25 ug/ml, 1.25 ug/ml and 0.31 ug/ml respectively, against the MPA. Membrane protein targets identified in screening were followed up in validation procedure using ligand serial dilution and cells individually transfected with identified targets.

### Example 5. Knockdown of mRNA in Muscle Cells Using CD71 FN3 Domain-Oligonucleotide Conjugates

muCD71 binding FN3 domains are conjugated to siRNA oligonucleotides or antisense oligonucleotides (ASOs) using maleimide chemistry via a cysteine that is uniquely engineered into the FN3 domain. The cysteine substitutions can be one such as those provided for herein and also as provided for in U.S. Patent Application Publication No. 20150104808, which is hereby incorporated by reference in its entirety. siRNAs or ASOs are modified with standard chemical modifications and confirmed to enable knockdown of the targeted mRNA in vitro. FN3 domain-oligonucleotide conjugates are dosed intravenously in mice at doses up to 10 mg/kg oligonucleotide payload. At various time points following dosing, mice are sacrificed; skeletal muscle, heart muscle and various other tissues will be recovered and stored in RNAlater™ (Sigma Aldrich) until needed. Target gene knockdown is assessed using standard qPCR ΔΔC_{T} methods and primers specific for the target gene and a control gene. The target gene is found to be knock downed in the muscles and such knockdown is enhanced by conjugating the siRNA or ASO to the CD71 FN3 binding domain.

### Example 6. Affinity Maturation Panning

4 sequences (A, B, C, and D) that demonstrated selective CD71 apical domain binding were the basis of affinity maturation library. In each sequence, 4 amino acids (double underlined), part of extended sheet library, were randomized to 18 amino acids (alanine, arginine, asparagine, aspartic acid, cysteine, glutamine, glutamic acid, glycine, histidine, isoleucine, leucine, lysine, phenylalanine, serine, threonine, tryptophan, tyrosine, or valine, not including proline, methionine). The new libraries underwent selection against 4 rounds of a) Transferrin wash; b) OKT9 elution; c) Apical domain selection; d) Apical domain, CD71_ECD selection. See sequences SEQ ID NOL 288-291 below.

| SEQ | |
|---|---|
| ID | |
| NO: | SEQUENCE |
| 288 | MLPAPKNLVVSRVTEDSARLSWTAPDAAFDSFYIAYAEPRPDGEAI |
| | VLTVPGSERSYDLTGLKPGTEYSVLIHGVKGGLLSSPLSAIFTT |
| 289 | MLPAPKNLVVSRVTEDSARLSWTAPDAAFDSFFIGYLEPQPPGEAI |
| | VLTVPGSERSYDLTGLKPGTEYNVTIQGVKGGFPSMPLSAIFTT |
| 290 | MLPAPKNLVVSRVTEDSARLSWTAPDAAFDSFHIVYHEPRPSGEAI |
| | VLTVPGSERSYDLTGLKPGTEYEVGIVSVKGGDLSVPLSAIFTT |
| 291 | MLPAPKNLVVSRVTEDSARLSWTAPDAAFDSFDIGYTEYGGYGEAI |
| | VLTVPGSERSYDLTGLKPGTEYWVLIQGVKGGGSSVPLSAIFTT |

TABLE 2
Summary of Screening Hits from the Apical domain
Panning: Primary Solution Elisa Screen

| hCD71, RLU | HSA, RLU | hCD71:HSA | SEQ ID |
|---|---|---|---|
| 1513450 | 15750 | 96 | 1 |
| 382750 | 28350 | 14 | 2 |
| 687350 | 9950 | 69 | 3 |
| 2097500 | 173650 | 12 | 4 |
| 1731850 | 44950 | 39 | 5 |
| 314750 | 7300 | 43 | 6 |
| 761250 | 26100 | 29 | 7 |
| 788350 | 29300 | 27 | 10 |
| 1243750 | 48950 | 25 | 12 |
| 1728000 | 24650 | 70 | 13 |
| 1404200 | 1100 | 1277 | 14 |
| 512000 | 14450 | 35 | 15 |
| 656900 | 6900 | 95 | 16 |
| 221400 | 3650 | 61 | 17 |
| 827750 | 10450 | 79 | 18 |
| 955800 | 17900 | 53 | 19 |
| 791200 | 18450 | 43 | 20 |
| 578950 | 6850 | 85 | 21 |
| 3153600 | 29950 | 105 | 22 |
| 1392100 | 12650 | 110 | 23 |
| 2452600 | 6550 | 374 | 24 |
| 221700 | 3850 | 58 | 25 |
| 460800 | 20750 | 22 | 26 |
| 297350 | 2350 | 127 | 27 |
| 382600 | 13900 | 28 | 28 |
| 2174900 | 3750 | 580 | 29 |
| 204900 | 4450 | 46 | 30 |
| 642000 | 4650 | 138 | 31 |
| 341550 | 4550 | 75 | 32 |
| 271350 | 2050 | 132 | 33 |
| 1575900 | 8100 | 195 | 34 |
| 1987300 | 18800 | 106 | 35 |
| 288400 | 4800 | 60 | 36 |
| 2946550 | 28600 | 103 | 37 |
| 517500 | 8150 | 63 | 38 |
| 1476450 | 51100 | 29 | 39 |
| 223200 | 13550 | 16 | 40 |
| 1374450 | 115000 | 12 | 41 |
| 586300 | 1000 | 586 | 42 |
| 772450 | 21500 | 36 | 43 |
| 714900 | 56650 | 13 | 44 |
| 393800 | 18450 | 21 | 45 |
| 1037700 | 8150 | 127 | 46 |
| 1986900 | 20350 | 98 | 47 |
| 3039800 | 114750 | 26 | 48 |
| 968550 | 13750 | 70 | 49 |
| 320700 | 15950 | 20 | 50 |
| 469100 | 41600 | 11 | 51 |
| 888200 | 15200 | 58 | 52 |
| 1271750 | 32050 | 40 | 53 |
| 578800 | 11700 | 49 | 54 |
| 670750 | 6800 | 99 | 55 |
| 1919800 | 19450 | 99 | 56 |
| 1115450 | 38800 | 29 | 57 |
| 1035000 | 10200 | 101 | 58 |
| 1111700 | 16450 | 68 | 59 |
| 472400 | 11650 | 41 | 60 |
| 252200 | 13550 | 19 | 61 |
| 229400 | 8100 | 28 | 62 |
| 4707950 | 5700 | 826 | 63 |
| 1310850 | 6000 | 218 | 64 |
| 959100 | 16050 | 60 | 65 |
| 232350 | 8100 | 29 | 66 |
| 1854450 | 14200 | 131 | 67 |
| 521350 | 38400 | 14 | 68 |
| 3170900 | 2700 | 1174 | 69 |
| 991450 | 94800 | 10 | 70 |
| 2643400 | 27500 | 96 | 71 |
| 338100 | 8950 | 38 | 72 |
| 1472250 | 20250 | 73 | 73 |
| 824500 | 14650 | 56 | 74 |
| 529750 | 3700 | 143 | 75 |
| 673950 | 16300 | 41 | 76 |
| 738800 | 10400 | 71 | 77 |
| 270900 | 9350 | 29 | 78 |
| 273400 | 6000 | 46 | 79 |
| 844350 | 33650 | 25 | 80 |
| 863750 | 9850 | 88 | 81 |
| 1555100 | 14850 | 105 | 82 |
| 3258100 | 110750 | 29 | 83 |
| 3526900 | 127150 | 28 | 84 |
| 210150 | 6350 | 33 | 85 |
| 624700 | 46500 | 13 | 86 |
| 281700 | 3650 | 77 | 87 |
| 515200 | 24150 | 21 | 88 |
| 918850 | 6500 | 141 | 89 |
| 564250 | 12350 | 46 | 90 |
| 491000 | 6600 | 74 | 91 |
| 528200 | 9850 | 54 | 92 |
| 430000 | 4750 | 91 | 93 |
| 1020750 | 87200 | 12 | 94 |
| 667050 | 40750 | 16 | 95 |
| 726000 | 15150 | 48 | 96 |
| 956900 | 6500 | 147 | 97 |
| 1305450 | 11200 | 117 | 98 |
| 440250 | 9850 | 45 | 99 |
| 408450 | 5150 | 79 | 100 |
| 335900 | 4600 | 73 | 101 |
| 443800 | 39700 | 11 | 102 |

TABLE 3
Summary of Screening Hits from the Affinity Maturation Panning:
Primary Solution Elisa Screen against CD71 and Apical Domain

| | Apical | | | | |
|---|---|---|---|---|---|
| hCD71, | domain, | HAS, | | apical | |
| RLU | RLU | RLU | hCD71:HSA | domain:HSA | SEQ ID |
| 817750 | 4141100 | 28950 | 28.2 | 143.0 | 103 |
| 947400 | 5902000 | 10550 | 89.8 | 559.4 | 104 |
| 2434450 | 6785550 | 17100 | 142.4 | 396.8 | 105 |
| 4966400 | 7370750 | 42000 | 118.2 | 175.5 | 106 |
| 1364850 | 5990050 | 62550 | 21.8 | 95.8 | 107 |
| 932300 | 2444500 | 13400 | 69.6 | 182.4 | 108 |
| 5327200 | 28300 | 38500 | 138.4 | 0.7 | 109 |
| 635550 | 802950 | 10600 | 60.0 | 75.8 | 110 |
| 2410700 | 112700 | 134450 | 17.9 | 0.8 | 111 |
| 4482200 | 7695300 | 10850 | 413.1 | 709.2 | 112 |
| 1266250 | 5832750 | 29700 | 42.6 | 196.4 | 113 |
| 2600900 | 6219500 | 21550 | 120.7 | 288.6 | 114 |
| 3470900 | 150050 | 172450 | 20.1 | 0.9 | 115 |
| 428500 | 4933450 | 11550 | 37.1 | 427.1 | 116 |
| 1296850 | 5357550 | 17200 | 75.4 | 311.5 | 117 |
| 1727250 | 6583250 | 14350 | 120.4 | 458.8 | 118 |
| 1246900 | 4926450 | 21650 | 57.6 | 227.5 | 119 |
| 501100 | 5745200 | 11800 | 42.5 | 486.9 | 120 |
| 4769200 | 6177350 | 17750 | 268.7 | 348.0 | 121 |
| 4769950 | 8057200 | 13600 | 350.7 | 592.4 | 122 |
| 1250400 | 4778850 | 29300 | 42.7 | 163.1 | 123 |
| 1539000 | 5460950 | 24400 | 63.1 | 223.8 | 124 |
| 1226400 | 5261600 | 16100 | 76.2 | 326.8 | 125 |
| 2735400 | 4416650 | 32200 | 85.0 | 137.2 | 126 |
| 1414600 | 6242100 | 21350 | 66.3 | 292.4 | 127 |
| 4308600 | 7406800 | 35500 | 121.4 | 208.6 | 128 |
| 218650 | 4179150 | 15850 | 13.8 | 263.7 | 129 |
| 1610200 | 105950 | 121850 | 13.2 | 0.9 | 130 |
| 203050 | 5277600 | 19650 | 10.3 | 268.6 | 131 |
| 1781950 | 7429250 | 41250 | 43.2 | 180.1 | 132 |
| 1044600 | 4739150 | 25500 | 41.0 | 185.8 | 133 |
| 639900 | 5387400 | 17450 | 36.7 | 308.7 | 134 |
| 967300 | 6048000 | 23000 | 42.1 | 263.0 | 135 |
| 3685900 | 4773100 | 23750 | 155.2 | 201.0 | 136 |
| 5108650 | 7866150 | 19100 | 267.5 | 411.8 | 137 |
| 1077450 | 5345250 | 14500 | 74.3 | 368.6 | 138 |
| 1133300 | 1860500 | 15350 | 73.8 | 121.2 | 139 |
| 603150 | 317150 | 21650 | 27.9 | 14.6 | 140 |
| 965000 | 4963650 | 26050 | 37.0 | 190.5 | 141 |
| 2965700 | 4364600 | 24100 | 123.1 | 181.1 | 142 |
| 3249450 | 7509300 | 20700 | 157.0 | 362.8 | 143 |
| 516500 | 3913550 | 16750 | 30.8 | 233.6 | 144 |
| 6196350 | 8295200 | 17800 | 348.1 | 466.0 | 145 |
| 1079650 | 5718700 | 16950 | 63.7 | 337.4 | 146 |
| 2939600 | 6887450 | 14400 | 204.1 | 478.3 | 147 |
| 289900 | 3411200 | 13750 | 21.1 | 248.1 | 148 |
| 1555800 | 6157400 | 17050 | 91.2 | 361.1 | 149 |
| 1300500 | 3157650 | 102550 | 12.7 | 30.8 | 150 |
| 1227350 | 4044500 | 27150 | 45.2 | 149.0 | 151 |
| 764200 | 4957500 | 13800 | 55.4 | 359.2 | 152 |
| 339200 | 3960750 | 21250 | 16.0 | 186.4 | 153 |
| 779200 | 5542450 | 23150 | 33.7 | 239.4 | 154 |
| 513250 | 4862050 | 18500 | 27.7 | 262.8 | 155 |
| 2455450 | 6708400 | 23500 | 104.5 | 285.5 | 156 |
| 3614600 | 7390500 | 38100 | 94.9 | 194.0 | 157 |
| 4992950 | 8005250 | 16000 | 312.1 | 500.3 | 158 |
| 185400 | 4433850 | 12850 | 14.4 | 345.0 | 159 |
| 4464400 | 8309700 | 22000 | 202.9 | 377.7 | 160 |
| 2632900 | 7310900 | 24650 | 106.8 | 296.6 | 161 |
| 638100 | 93100 | 37500 | 17.0 | 2.5 | 162 |
| 312700 | 74500 | 27150 | 11.5 | 2.7 | 163 |
| 3781250 | 8417150 | 25650 | 147.4 | 328.2 | 164 |
| 4906500 | 8370450 | 28000 | 175.2 | 298.9 | 165 |
| 1220000 | 6080200 | 35850 | 34.0 | 169.6 | 166 |
| 4173000 | 128250 | 179800 | 23.2 | 0.7 | 167 |
| 3972000 | 6860300 | 14650 | 271.1 | 468.3 | 168 |
| 4136300 | 7211350 | 17150 | 241.2 | 420.5 | 169 |
| 813500 | 4861500 | 41400 | 19.6 | 117.4 | 170 |
| 653400 | 5475650 | 18500 | 35.3 | 296.0 | 171 |
| 376600 | 353200 | 28200 | 13.4 | 12.5 | 172 |
| 1486150 | 1796200 | 22350 | 66.5 | 80.4 | 173 |
| 1899250 | 4829350 | 30800 | 61.7 | 156.8 | 174 |
| 4321900 | 7163900 | 13800 | 313.2 | 519.1 | 175 |
| 730600 | 5926750 | 9500 | 76.9 | 623.9 | 176 |
| 2796650 | 7737350 | 34850 | 80.2 | 222.0 | 177 |
| 865300 | 4948100 | 19250 | 45.0 | 257.0 | 178 |
| 3679650 | 7294100 | 28900 | 127.3 | 252.4 | 179 |
| 652900 | 3879150 | 10600 | 61.6 | 366.0 | 180 |
| 942200 | 6151700 | 15200 | 62.0 | 404.7 | 181 |
| 197350 | 2843300 | 13600 | 14.5 | 209.1 | 182 |
| 1128400 | 6952050 | 22450 | 50.3 | 309.7 | 183 |
| 4040600 | 119550 | 145000 | 27.9 | 0.8 | 184 |
| 651650 | 5458200 | 14800 | 44.0 | 368.8 | 185 |
| 844300 | 5382550 | 16700 | 50.6 | 322.3 | 186 |
| 796000 | 77900 | 73100 | 10.9 | 1.1 | 187 |
| 357550 | 5244350 | 26950 | 13.3 | 194.6 | 188 |
| 2567500 | 98900 | 119150 | 21.5 | 0.8 | 189 |
| 384850 | 4892250 | 23500 | 16.4 | 208.2 | 190 |
| 3864050 | 4656300 | 32500 | 118.9 | 143.3 | 191 |
| 1769550 | 7584950 | 22150 | 79.9 | 342.4 | 192 |
| 1711550 | 6118550 | 27950 | 61.2 | 218.9 | 193 |
| 682500 | 4781900 | 17500 | 39.0 | 273.3 | 194 |
| 4265600 | 7351800 | 41550 | 102.7 | 176.9 | 195 |
| 2133100 | 5927950 | 25200 | 84.6 | 235.2 | 196 |
| 1395000 | 6777950 | 47200 | 29.6 | 143.6 | 197 |
| 3905250 | 7195200 | 21050 | 185.5 | 341.8 | 198 |
| 416250 | 5555950 | 17150 | 24.3 | 324.0 | 199 |
| 929650 | 6103250 | 18000 | 51.6 | 339.1 | 200 |
| 3249150 | 7496450 | 232850 | 14.0 | 32.2 | 201 |
| 1508700 | 5468850 | 25750 | 58.6 | 212.4 | 202 |
| 2378300 | 5694300 | 25950 | 91.6 | 219.4 | 203 |
| 3285350 | 6352000 | 20050 | 163.9 | 316.8 | 204 |
| 2805100 | 150050 | 155300 | 18.1 | 1.0 | 205 |
| 827850 | 5220700 | 14000 | 59.1 | 372.9 | 206 |
| 2735800 | 7111400 | 120850 | 22.6 | 58.8 | 207 |
| 3001350 | 3698200 | 16350 | 183.6 | 226.2 | 208 |
| 6196350 | 8295200 | 17800 | 348.1 | 466.0 | 209 |
| 2411400 | 4124450 | 48550 | 49.7 | 85.0 | 210 |
| 660100 | 4739550 | 13050 | 50.6 | 363.2 | 211 |
| 466900 | 5090650 | 17450 | 26.8 | 291.7 | 212 |
| 1727000 | 2030750 | 70300 | 24.6 | 28.9 | 213 |
| 393150 | 4299750 | 30150 | 13.0 | 142.6 | 214 |
| 2020450 | 5842350 | 22350 | 90.4 | 261.4 | 215 |
| 4147550 | 141900 | 191700 | 21.6 | 0.7 | 216 |
| 3475050 | 6812350 | 16200 | 214.5 | 420.5 | 217 |
| 143600 | 2845650 | 14100 | 10.2 | 201.8 | 218 |
| 459750 | 4948500 | 11850 | 38.8 | 417.6 | 219 |
| 965600 | 31750 | 58950 | 16.4 | 0.5 | 221 |
| 1468850 | 1073450 | 15550 | 94.5 | 69.0 | 222 |
| 1929050 | 3597800 | 18300 | 105.4 | 196.6 | 223 |
| 3547950 | 6822800 | 26150 | 135.7 | 260.9 | 224 |
| 4057750 | 7377100 | 16950 | 239.4 | 435.2 | 225 |
| 2154200 | 6376550 | 15950 | 135.1 | 399.8 | 226 |
| 634750 | 4992500 | 28200 | 22.5 | 177.0 | 227 |
| 3881750 | 7042200 | 29550 | 131.4 | 238.3 | 228 |
| 1443150 | 5772800 | 15450 | 93.4 | 373.6 | 229 |
| 1100650 | 6105450 | 32000 | 34.4 | 190.8 | 230 |
| 2322100 | 6834550 | 20650 | 112.5 | 331.0 | 231 |
| 1079350 | 95450 | 75400 | 14.3 | 1.3 | 232 |
| 771600 | 5605650 | 22000 | 35.1 | 254.8 | 233 |
| 4506100 | 7037600 | 20850 | 216.1 | 337.5 | 234 |
| 943050 | 1672000 | 23850 | 39.5 | 70.1 | 235 |
| 4071550 | 7411950 | 15550 | 261.8 | 476.7 | 236 |
| 4576600 | 7006350 | 27700 | 165.2 | 252.9 | 237 |
| 3069350 | 111900 | 98950 | 31.0 | 1.1 | 238 |
| 559400 | 4653950 | 21100 | 26.5 | 220.6 | 239 |
| 610150 | 5256450 | 18000 | 33.9 | 292.0 | 240 |
| 3100050 | 5054100 | 26550 | 116.8 | 190.4 | 241 |
| 648150 | 4898500 | 20350 | 31.9 | 240.7 | 242 |
| 1972450 | 3193200 | 30900 | 63.8 | 103.3 | 243 |
| 1747650 | 5926450 | 18000 | 97.1 | 329.2 | 244 |
| 773800 | 4773600 | 18450 | 41.9 | 258.7 | 245 |
| 548350 | 47500 | 25750 | 21.3 | 1.8 | 246 |
| 4298600 | 7512300 | 19050 | 225.6 | 394.3 | 247 |
| 755550 | 5450050 | 34000 | 22.2 | 160.3 | 248 |
| 4935550 | 7432850 | 24100 | 204.8 | 308.4 | 249 |
| 724450 | 5544450 | 48450 | 15.0 | 114.4 | 250 |
| 2508550 | 5734400 | 26100 | 96.1 | 219.7 | 251 |
| 4344000 | 7572700 | 72850 | 59.6 | 103.9 | 252 |
| 3469100 | 7212700 | 37700 | 92.0 | 191.3 | 253 |
| 2239400 | 6619100 | 21500 | 104.2 | 307.9 | 254 |
| 5021750 | 7080200 | 102200 | 49.1 | 69.3 | 255 |
| 2071450 | 5839600 | 76100 | 27.2 | 76.7 | 256 |
| 802300 | 4835650 | 29800 | 26.9 | 162.3 | 257 |
| 5396000 | 7340850 | 26500 | 203.6 | 277.0 | 258 |
| 1259150 | 5961350 | 25350 | 49.7 | 235.2 | 259 |
| 4875150 | 7054650 | 33400 | 146.0 | 211.2 | 260 |
| 2393600 | 93150 | 174150 | 13.7 | 0.5 | 261 |
| 3941300 | 7126200 | 30900 | 127.6 | 230.6 | 262 |
| 584250 | 4370450 | 23800 | 24.5 | 183.6 | 263 |
| 368350 | 4322150 | 24400 | 15.1 | 177.1 | 264 |
| 5772300 | 7208200 | 30700 | 188.0 | 234.8 | 265 |
| 418150 | 4232150 | 36150 | 11.6 | 117.1 | 266 |
| 573450 | 5545650 | 29850 | 19.2 | 185.8 | 267 |
| 2767600 | 94850 | 117300 | 23.6 | 0.8 | 268 |
| 812000 | 5559700 | 19900 | 40.8 | 279.4 | 269 |
| 3320500 | 6487650 | 64350 | 51.6 | 100.8 | 270 |

TABLE 4
Summary of Size Exclusion Chromatography Analysis
of Hits from the Apical domain panning

| SEQ ID | RT (min) | Height (mAU) | Y/N |
|---|---|---|---|
| 1 | 4.85 | 1276 | N |
| 2 | 6.18 | 5190 | Y |
| 3 | 4.49 | 12962 | N |
| 4 | 4.94 | 4430 | N |
| 5 | 5.76 | 72277 | Y |
| 6 | 4.42 | 9816 | N |
| 7 | 5.80 | 6831 | N |
| 10 | 4.42 | 13626 | N |
| 12 | 5.19 | 17868 | N |
| 13 | 5.02 | 7274 | Y |
| 14 | 5.83 | 49446 | Y |
| 15 | 4.43 | 16661 | N |
| 16 | 4.42 | 14549 | N |
| 17 | 4.47 | 12403 | N |
| 18 | 4.41 | 11175 | N |
| 19 | 4.43 | 12918 | N |
| 20 | 4.41 | 31082 | N |
| 21 | 5.75 | 50073 | Y |
| 22 | 6.02 | 24897 | Y |
| 23 | 4.42 | 12349 | N |
| 24 | 5.35 | 12795 | N |
| 25 | 4.45 | 14147 | Y |
| 26 | 5.81 | 11762 | N |
| 27 | 4.41 | 12376 | N |
| 28 | 5.79 | 6468 | N |
| 29 | 4.42 | 14375 | N |
| 30 | 5.90 | 31537 | N |
| 31 | 5.78 | 64734 | Y |
| 32 | 4.48 | 11824 | N |
| 33 | 4.43 | 14874 | N |
| 34 | 4.40 | 16722 | N |
| 35 | 4.43 | 30118 | N |
| 36 | 4.43 | 10366 | N |
| 37 | 5.91 | 10078 | N |
| 38 | 5.77 | 65938 | Y |
| 39 | 5.73 | 21308 | N |
| 40 | 5.78 | 9542 | N |
| 41 | 5.84 | 51999 | Y |
| 42 | 5.99 | 68358 | Y |
| 43 | 4.85 | 103763 | N |
| 44 | 6.11 | 39261 | N |
| 45 | 6.04 | 107908 | Y |
| 46 | 4.89 | 6108 | Y |
| 47 | 5.97 | 4414 | N |
| 48 | 6.16 | 40159 | Y |
| 49 | 6.04 | 95232 | Y |
| 50 | 4.49 | 10016 | N |
| 51 | 5.82 | 9242 | N |
| 52 | 4.48 | 15396 | N |
| 53 | 4.47 | 11234 | N |
| 54 | 4.43 | 15089 | N |
| 55 | 5.93 | 45271 | Y |
| 56 | 4.43 | 21968 | N |
| 57 | 4.48 | 9865 | N |
| 58 | 5.84 | 27831 | N |
| 59 | 4.49 | 15581 | N |
| 60 | 4.44 | 15771 | N |
| 61 | 4.44 | 16491 | N |
| 62 | 4.46 | 12971 | N |
| 63 | 5.84 | 55413 | Y |
| 64 | 5.76 | 66310 | Y |
| 65 | 5.74 | 13334 | N |
| 66 | 5.74 | 31088 | N |
| 67 | 4.43 | 24872 | N |
| 68 | 5.85 | 68979 | Y |
| 69 | 5.76 | 54708 | Y |
| 70 | 5.85 | 57285 | Y |
| 71 | 4.45 | 11624 | N |
| 72 | 4.47 | 17529 | N |
| 73 | 5.89 | 115880 | Y |
| 74 | 4.50 | 11311 | N |
| 75 | 5.84 | 33371 | Y |
| 76 | 4.49 | 19602 | N |
| 77 | 5.74 | 61741 | Y |
| 78 | 5.81 | 51210 | Y |
| 79 | 4.48 | 14307 | N |
| 80 | 5.87 | 82953 | Y |
| 81 | 4.49 | 11000 | N |
| 82 | 4.50 | 9074 | N |
| 83 | 5.73 | 27551 | N |
| 84 | 5.86 | 108824 | Y |
| 85 | 5.82 | 70054 | Y |
| 86 | 5.80 | 7988 | N |
| 87 | 4.47 | 12777 | N |
| 88 | 5.80 | 88617 | Y |
| 89 | 5.80 | 14227 | N |
| 90 | — | — | N |
| 91 | 4.45 | 19485 | N |
| 92 | 5.85 | 15361 | N |
| 93 | 5.994 | 7470 | Y |
| 94 | 6.045 | 4967 | N |
| 95 | 6.011 | 2105 | N |
| 96 | 4.694 | 2993 | N |
| 97 | 5.982 | 5411 | N |
| 98 | 6.086 | 15055 | Y |
| 99 | 6.127 | 5459 | Y |
| 100 | 4.764 | 1593 | N |
| 101 | 4.799 | 1552 | N |
| 102 | 5.971 | 1289 | N |

TABLE 5
Summary of Size Exclusion Chromatography Analysis
of Hits from the Affinity Maturation panning

| SEQ ID | RT (min) | Height (mAU) | Y/N |
|---|---|---|---|
| 103 | 5.88 | 138095 | Y |
| 104 | 5.89 | 130922 | Y |
| 105 | 5.81 | 89766 | Y |
| 106 | 5.80 | 176348 | Y |
| 107 | 5.91 | 101236 | Y |
| 108 | 5.75 | 115796 | Y |
| 109 | 5.83 | 142833 | Y |
| 110 | 5.78 | 94728 | Y |
| 111 | 5.12 | 35930 | N |
| 112 | 5.81 | 109962 | Y |
| 113 | 5.09 | 106964 | Y |
| 114 | 5.84 | 104762 | Y |
| 115 | 5.81 | 38373 | N |
| 116 | 5.81 | 104287 | Y |
| 117 | 5.76 | 115912 | Y |
| 118 | 5.87 | 138528 | Y |
| 119 | 5.74 | 119491 | Y |
| 120 | 5.82 | 105691 | Y |
| 121 | 5.78 | 110048 | Y |
| 122 | 5.83 | 158539 | Y |
| 123 | 5.74 | 111953 | Y |
| 124 | 5.94 | 114526 | Y |
| 125 | 5.92 | 137742 | Y |
| 126 | 5.79 | 112960 | Y |
| 127 | 5.86 | 110390 | Y |
| 128 | 5.90 | 120352 | Y |
| 129 | 5.89 | 134927 | Y |
| 130 | 5.81 | 118419 | Y |
| 131 | 5.91 | 166662 | Y |
| 132 | 5.88 | 119689 | Y |
| 133 | 5.82 | 132427 | Y |
| 134 | 5.83 | 114205 | Y |
| 135 | 5.92 | 103105 | Y |
| 136 | 5.71 | 98649 | Y |
| 137 | 5.75 | 117138 | Y |
| 138 | 5.91 | 106938 | Y |
| 139 | 5.77 | 86558 | Y |
| 140 | 5.74 | 96522 | Y |
| 141 | 5.78 | 95668 | Y |
| 142 | 5.88 | 73328 | Y |
| 143 | 6.00 | 83733 | Y |
| 144 | 5.87 | 100941 | Y |
| 145 | 5.82 | 109668 | Y |
| 146 | 5.87 | 99569 | Y |
| 147 | 5.75 | 120095 | Y |
| 148 | 5.83 | 129029 | Y |
| 149 | 5.91 | 130589 | Y |
| 150 | 4.91 | 30926 | N |
| 151 | 5.81 | 95796 | Y |
| 152 | 5.85 | 141203 | Y |
| 153 | 5.93 | 106098 | Y |
| 154 | 5.94 | 160425 | Y |
| 155 | 5.90 | 143145 | Y |
| 156 | 5.86 | 115893 | Y |
| 157 | 5.85 | 100355 | Y |
| 158 | 5.83 | 126349 | Y |
| 159 | 5.84 | 158223 | Y |
| 160 | 5.84 | 148628 | Y |
| 161 | 5.82 | 138546 | Y |
| 162 | 5.73 | 57532 | N |
| 163 | 5.82 | 39335 | N |
| 164 | 5.84 | 117968 | Y |
| 165 | 5.84 | 149834 | Y |
| 166 | 5.89 | 114338 | Y |
| 167 | 5.17 | 30228 | N |
| 168 | 5.77 | 127513 | Y |
| 169 | 5.82 | 100417 | Y |
| 170 | 5.92 | 132112 | Y |
| 171 | 5.89 | 67901 | N |
| 172 | 5.78 | 86348 | Y |
| 173 | 5.81 | 119484 | Y |
| 174 | 5.83 | 80373 | Y |
| 175 | 5.84 | 128180 | Y |
| 176 | 5.90 | 163155 | Y |
| 177 | 5.84 | 43159 | Y |
| 178 | 5.83 | 125240 | Y |
| 179 | 5.77 | 119270 | Y |
| 180 | 5.95 | 110362 | Y |
| 181 | 4.81 | 190088 | Y |
| 182 | 5.81 | 151104 | Y |
| 183 | 5.77 | 143551 | Y |
| 184 | 4.94 | 40376 | N |
| 185 | 5.90 | 117671 | Y |
| 186 | 5.80 | 119592 | Y |
| 187 | 4.91 | 59493 | N |
| 188 | 5.80 | 126167 | Y |
| 189 | 5.83 | 173711 | Y |
| 190 | 5.75 | 132569 | Y |
| 191 | 5.82 | 129102 | Y |
| 192 | 5.86 | 185255 | Y |
| 193 | 5.82 | 110511 | Y |
| 194 | 5.94 | 101317 | Y |
| 195 | 5.77 | 117916 | Y |
| 196 | 5.88 | 122474 | Y |
| 197 | 5.84 | 103601 | Y |
| 198 | 5.81 | 132253 | Y |
| 199 | 5.89 | 118621 | Y |
| 200 | 5.84 | 179035 | Y |
| 201 | 5.90 | 162216 | Y |
| 202 | 6.04 | 41767 | Y |
| 203 | 6.02 | 14216 | Y |
| 204 | 5.21 | 10597 | N |
| 205 | 5.27 | 4282 | N |
| 206 | 6.06 | 14434 | Y |
| 207 | 6.07 | 16590 | Y |
| 208 | 5.05 | 14071 | Y |
| 209 | 5.82 | 109668 | Y |
| 210 | 5.97 | 15408 | Y |
| 211 | 6.17 | 13650 | Y |
| 212 | 6.10 | 22359 | Y |
| 213 | 5.92 | 3377 | N |
| 214 | 6.07 | 20042 | Y |
| 215 | 6.14 | 20768 | Y |
| 216 | 5.25 | 5654 | N |
| 217 | 5.98 | 20229 | Y |
| 218 | 6.00 | 14717 | Y |
| 219 | 6.00 | 13928 | Y |
| 221 | 5.20 | 9340 | N |
| 222 | 5.98 | 15512 | Y |
| 223 | 6.01 | 12837 | Y |
| 224 | 6.13 | 12868 | Y |
| 225 | 6.02 | 18790 | Y |
| 226 | 6.13 | 15649 | Y |
| 227 | 6.14 | 13107 | Y |
| 228 | 6.05 | 18737 | Y |
| 229 | 6.17 | 15369 | Y |
| 230 | 6.11 | 26740 | Y |
| 231 | 5.99 | 14844 | Y |
| 232 | 5.19 | 9635 | N |
| 233 | 6.10 | 22603 | Y |
| 234 | 6.04 | 15360 | Y |
| 235 | 5.95 | 12764 | Y |
| 236 | 5.99 | 16948 | Y |
| 237 | 6.05 | 17113 | Y |
| 238 | 5.22 | 9826 | N |
| 239 | 6.05 | 19054 | Y |
| 240 | 5.99 | 11335 | Y |
| 241 | 6.02 | 12881 | Y |
| 242 | 6.15 | 15577 | Y |
| 243 | 5.98 | 11788 | Y |
| 244 | — | — | N |
| 245 | 6.15 | 17079 | Y |
| 246 | 6.14 | 113391 | Y |
| 247 | 6.02 | 13674 | Y |
| 248 | 6.10 | 19453 | Y |
| 249 | 5.98 | 17677 | Y |
| 250 | 6.07 | 13344 | Y |
| 251 | 6.13 | 16320 | Y |
| 252 | 6.05 | 14756 | Y |
| 253 | 6.05 | 16634 | Y |
| 254 | 6.02 | 14227 | Y |
| 255 | 6.03 | 12605 | Y |
| 256 | 6.03 | 16075 | Y |
| 257 | 6.02 | 16086 | Y |
| 258 | 6.02 | 15720 | Y |
| 259 | 6.03 | 12004 | Y |
| 260 | 5.99 | 14310 | Y |
| 261 | 5.99 | 6832 | Y |
| 262 | 6.04 | 13901 | Y |
| 263 | 6.03 | 15172 | Y |
| 264 | 6.00 | 13075 | Y |
| 265 | 5.97 | 10535 | Y |
| 266 | 5.98 | 12563 | Y |
| 267 | 6.17 | 18078 | Y |
| 268 | 6.05 | 9392 | N |
| 269 | — | — | N |
| 270 | 4.78 | 2974 | N |

Sequences of Hits from the Apical Domain Panning

| SEQ | |
|---|---|
| ID | Amino Acid sequence of FN3 domains that bind to CD71 |
| 1 | MLPAPKNLVVSRVTEDSARLSWTAPDAAFDSFPITYIEAVVLGEAIVLTVPGSER |
| | SYDLTGLKPGTEYPVGISGVKGGHNSMPLSAIFTT |
| 2 | MLPAPKNLVVSEVTEDSARLSWQGVARAFDSFMINYSELFWMGEAIVLTVPGS |
| | ERSYDLTGLKPGTEYVVRIKGVKGGKGSWPLHAHFTT |
| 3 | MLPAPKNLVVSRVTEDSARLSWTAPDAAFDSFNIEYAETRWYGEAIVLTVPGSE |
| | RSYDLTGLKPGTEYVVPIDGVKGGIASKPLSAIFTT |
| 4 | MLPAPKNLVVSEVTEDSARLSWQGVARAFDSFLITYRDQIFAGEVIVLTVPGSE |
| | RSYDLTGLKPGTEYPVWIQGVKGGSPSAPLSAESTT |
| 5 | MLPAPKNLVVSEVTEDSARLSWQGVARAFDSFLITYREQIFAGEVIVLTVPGSER |
| | SYDLTGLKPGTEYWVYIWGVKGGKPSFPLRAGFTT |
| 6 | MLPAPKNLVVSRVTEDSARLSWTAPDAAFDSFPIIYMETFSRGEAIVLTVPGSER |
| | SYDLTGLKPGTEYRVPIGGVKGGSSSCPLSAIFTT |
| 7 | MLPAPKNLVVSDVTEDSARLSWQGVARAFDSFLITYREQIFAGEVIVLTVPGSE |
| | RSYDLTGLKPGTEYPVWIQGVKGGSPSAPLSAEFTT |
| 10 | MLPAPKNLVVSRVTEDSARLSWTAPDAAFDSFKIAYIETATRGEAIVLTVPGSER |
| | SYDLTGLKPGTEYVVPIPGVKGGNTSSPLSAIFTT |
| 12 | MLPAPKNLVVSRVTEDSARLSWTAPDAAFDSFFIPYAEPSPTGEAIVLTVPGSER |
| | SYDLTGLKPGTEYSVLIHGVKGGHLSDPLSAISTT |
| 13 | MLPAPKNLVVSRVTEDSARLSWTAPDAAFDSFLIAYPEDGFRGEAIVLTVPGSE |
| | RSYDLTGLKPGTEYPVPILGVKGGGGSGPLSAIFTT |
| 14 | MLPAPKNLVVSRVTEDSARLSWTAPDAAFDSFPIYYVENVVWGEAIVLTVPGSE |
| | RSYDLTGLKPGTEYWEVIIGVKGGQCSRPLSAIFTT |
| 15 | MLPAPKNLVVSRVTEDSARLSWQGVARAFDSFLITYREQIFAGEVIVLTVPGSER |
| | SYDLTGLKPGTECPVWIQGVKGGSPSAPLSAEFTT |
| 16 | MLPAPKNLVVSRVTEDSARLSWTAPDAAFDSFGIAYREFRPSGEAIVLTVPGSER |
| | SYDLTVETGYRNEVVICGVKGGPWSGPLSAIFTT |
| 17 | MLPAPKNLVVSRVTEDSARLSWTAPDAAFDSFPILYTECVYRGEAIVLTVPGSE |
| | RSYDLTGLKPGTEYHVPITGVKGGGGSWPLSAIFTT |
| 18 | MLPAPKNLVVSRVTEDSARLSWTAPDAAFDSFNIMYHEIIYVGEAIVLTVPGSER |
| | SYDLTGLKPGTEYPVPIEGVKGGGTSGPLSAIFTT |
| 19 | MLPAPKNLVVSRVTEDSARLSWTAPDAAFDSFAITYTEAALCGEAIVLTVPGSE |
| | RSYDLTGLKPGTEYPVPINGVKGGGTSGPLSAIFTT |
| 20 | MLPAPKNLVVARVTEDSARLSWTAPDAAIDSFPIDYSEYWWGGEAIVLTVPGSE |
| | RSYDLTGLKPGTEYPVLITGVKGGYRSGPLSAIFTT |
| 21 | MLPAPKNLVVSRVTEDSARLSWTAPDAAFDSFSIRYNEFIVAGEAIVLTVPGSER |
| | SYDLTGLKPGTEYDVPIAGVKGGGASWPLSAIVTT |
| 22 | MLPAPKNLVVSRVTEDSARLSWTAPDAAFDSFWYLELQFAGEAIVLTVPGSERS |
| | YDLTGLKPGTEYNVPITGVKGGIISFPLSAIFTT |
| 23 | MLPAPKNLVVSRVTEDSARLSWTAPDAAFDSFAIWYHEWYGDGEAIVLTVPGS |
| | ERSYDLTGPKPGTEYRVRISGVKGGFESGPLSAIFTT |
| 24 | MLPAPKNLVVSRVTEDSARLSWTAPDAAFDSFMIRYQEGTRWGEAIVLTVPGS |
| | ERSYDLTGLKPGTEYIVMIAGVKGGQISLPLSAIFTT |
| 25 | MLPAPKNLVVSRVTEDSARLSWTAPDAAFDSFPIWYLEKSYQGEAIVLTVPGSE |
| | RSYDLTGLKPGTEYVVPIIGVKGGRDSCPLSAIFTT |
| 26 | MLPAPKNLVVSEVTEDSARLSWQGVARAFDSFLITYREQIFAGEVIVLTVPGSER |
| | SYDLTGLKPGTEYPVWIQGVKGGSPSAPLSAEFTT |
| 27 | MLPAPKNLVVSRVTEDSARLSWTAPDAAFDSFRISYAETVRQGEAIVLTVPGSE |
| | RSYDLTVETGYRNWVMILGVKGGPGSLPLSAIFTT |
| 28 | MLPAPKNLVVSEVTEDSARLSWQGVVRAFDSFLITYREQIFAGEVIVLTVPGSER |
| | SYDLTGLKPGTEYPVWIQGVKGGSPSAPLSAEFTT |
| 29 | MLPAPKNLVVSRVTEDSARLSWTAPDAAFDSFWIEYWEAVGFGEAIVLTVPGS |
| | ERSYDLTGLKPGTEYFVGIYGVKGGYLSAPLSAIFTT |
| 30 | MLPAPKNLVVSRVTEDSARLSWTAPDAAFDSFIIHYVEQQLIGEAIVLTVPGSER |
| | SYDLTGLKPGTEYPVPITGVKGGACSWPLSAIFTT |
| 31 | MLPAPKNLVVSRVTEDSARLSWTAPDAAFDSFTIEYSEHPIDGEAIPLFVPGSERS |
| | YDLTGLKPGTEYYVRIHGVKGGWFSHPLWAFFTT |
| 32 | MLPAPKNLVVSRVTEDSARLSWQGVARAFDSFLITYREQIFAGEVIVLTVPGSER |
| | SYDLTGLKPGTEYGVTIAGVKGGWRSKPLNAESTT |
| 33 | MLPAPKNLVVSRVTEDSARLSWTAPDAAFDSFGIAYVESYWYGEAIVLTVPGSE |
| | RSYDLTGLKPGTEYNVPIYGVKGGDGSGPLSAIFTT |
| 34 | MLPAPKNLVVSRVTEDSARLSWTAPDAAFDSFYITYVELNLAGEAIVLTVPGSE |
| | RSYDLTGLKPGTEYPVPILGVKGGSLSQPLSAIFTT |
| 35 | MLPAPKNLVVSRVTEDSARLSWTAPDAAFDSFPISYIESIADGEAIVLTVPGSERS |
| | YDLTGLKPGTEYWVAIVGVKGGPFSWSLSAIVTT |
| 36 | MLPAPKNLVVSEVTEDSARLSWQGVARAFDSFLITYREQIFAGEVIVPTVPGSER |
| | SYDLTGLKPGTEYPVPIAGVKGGGPSAPLSAIFTT |
| 37 | MLPAPKNLVVSRVTEDSARLSWTTPDAAFDSFPIYYWEVTITGEAIYLSVPGSER |
| | SYDLTGLKPGTEYPVDIPGVKGGAASPPLSAIFTT |
| 38 | MLPAPKNLVVSRVTEDSARLSWTAPDAAFDSFPILYLEHTVRSEAIVLTVPGSER |
| | SYDLTDLKPGTEYCVPIDGVKGGLRSRPLSAIFTT |
| 39 | MLPAPKNLVVSRVTEDSARLSWTAPDAAFDSFPIPYTEPPDPGEAIVLTVPGSER |
| | SYDLTGLKPGTEYLVTILGVKGGSMSVPLSAIFTT |
| 40 | MLPAPKNLVVSRVTEDSARLSWTAPDAAFDSFTIDYWENRCPGEAIVLTVPGSE |
| | RSYDLTGLKPGTEYCVWISGVKGGYSSWPLSAIFTT |
| 41 | MLPAPKNLVVSRVTEDSARLSWQGVARAFDSFLITYREQIFAGEVIVLTVPGSER |
| | SYDLTGLKPGTEYPVWIQGVKGGHLSDPLSAIVTT |
| 42 | MLPAPKNLVVSRVTEDSARLSWTAPDAAFDSFLIPYAETSPSGEAIVLTVPGSER |
| | SYDLTGLKPGTEYSVLIHGVKGGDYSEPLSAIFTT |
| 43 | MLPAPKNLVVSRVTEDSARLSWTAPDAAFDSFMIVYYEYTRFGEAIVLTVPGSE |
| | RSYDLTGLKPGTEYTVPIDGVKGGGRSSPLSAIFTT |
| 44 | MLPAPKNLVVSRVTEDSARLSWTAPDAAFDSFFIPYAEPSPTGEAIVLTVPGSER |
| | SYDLTGLKPGTEYSVLIHGVKGGHLSDPLSAIVTT |
| 45 | MLPAPKNLVVSEVTEDSARLSWQGVARAFDSFLITYREQIFAGEVIVLTVPGSER |
| | SYDLTGLKPGTEYPVWIQGVKGGSPSAPLSAEFTT |
| 46 | MLPAPKNLVVSRVTEDSARLSWTAPDAAFDSFIIPYAEVRPDGEAIVLTVPGSER |
| | SYDLTGLKPGTEYSVLIHGVKGGKLSLPLSAIFTT |
| 47 | MLPAPKNLVVSRVTEDSARLSWTAPDAAFDSFGIVYLEMMVTGEAIVLTVPGS |
| | ERSYDLTGLKPGTEYDVPILGVKGGTRSVPLSAIFTT |
| 48 | MLPAPKNLVVSRVTEDSARLSWTAPDAAFDSFLIYYEEGYLEYYYSGEAIVLTV |
| | PGSERS YDLTGLKPGTEYYVGIVGVKGGGLSGPLSAISTT |
| 49 | MLPAPKNLVVSRVTEDSARLSWTAPDAAFDSFPIAYAEPRPDGEAIVLTVPGSE |
| | RSYDLTGLKPGTEYSVLIHGVKGGDWSLPLSAIFTT |
| 50 | MSLPAPKNLVVSRVTEDSARLSWTAPDAAFDSFTIHYREFQLSGEAIVLTVPGSE |
| | RSYDLTGLKPGTEYDVPIEGVKGGPGSRPLSAIFTT |
| 51 | MLPAPKNLVVSEVTEDSARLSWQGVARAFDSFLITYREQIFAGEVIVLTVPGSEC |
| | SYDLTGLKPGTEYPVWIQGVKGGSPSAPLSAEFTT |
| 52 | MLPAPKNLVVSRVTEDSARLSWTAPDAAFDSFEIDYDELAIYGEAIVLTVPGSER |
| | SYDLTGLKPGTEYGVRIPGVKGGMPSLPLSAIVTT |
| 53 | MLPAPENLVVSEVTEDSARLSWQGVARAFDSFLITYREQIFAGEVIVLTVPGSER |
| | SYDLTGLKPGTEYPVWIQGVKGGSPSAPLSAESTT |
| 54 | MLPAPKNLVVSRVTEDSARLSWTAPDAAFDSFAIAYGEHIVIGEAIVLTVPGSER |
| | SYDLTGLKPGTEYMVPIAGVKGGPISLPLSAIFTT |
| 55 | MLPAPKNLVVSRVTEDSARLSWTAPDAAFDSFFIPYAEPSPTGEAIVLTVPGSER |
| | SYDLTGLKPGTEYSVLIHGVKGGHLSDPLSAIFTT |
| 56 | MLPAPKNLVVSRVTEDSARLSWTAPDAAFDSFSIGYVELVLLGEAIVLTVPGSE |
| | RSYDLTGLKPGTEYDVLIPGVKGGSLSRPLSAIFTT |
| 57 | MLPAPKNLVVSRVTEDSARLSWTAPDAAFDSFAIPYAELSRNGEAIVLTVPGSE |
| | RSYDLTGLKPGTEYTVLIHGVKGGCLSDPLSAIFTT |
| 58 | MLPAPKNLVVSRVTEDSARLSWTAPDAAFDSFHIEYLELSRHGEAIVLTVPGSE |
| | RSYDLTGLKPGTEYWVMIFGVKGGGPSKPLSAIFTT |
| 59 | MLPAPKNLVVSRVTEDSARLSWTAPDAAFDSFVYNEVHWIGEAIVLTVPGSERS |
| | YDLTGLKPGTEYFVGIYGVKGGHWSKPLSAIFTT |
| 60 | MLPAPKNLVVSRVTEDSARLSWTAPDAAFDSFEIDYDELAIYGEAIVLTVPGSER |
| | SYDLTGLKPGTEYGVRIPGVKGGMPSLPLSAIVTT |
| 61 | MLPAPKNLVVSRVTEDSARLSWTAPDAAFDSFQIVYSELWIKGEAIVLTVPGSE |
| | RSYDLTGLKPGTEYQVPIPGVKGGRNSFPLSAIFTT |
| 62 | MLPAPKNLVVSRVTEDSARLSWTAPDAAFDSFKIRYTETRSIGEAIVLTVPGSER |
| | SYDLTGLKPGTEYCVPIGGVKGGDSSWPLSAISTT |
| 63 | MLPAPKNLVVSRVTEDSARLSWTAPDAAFDSFCISYYERMGRGEAIVLTVPGSE |
| | RSYDLTGLKPGTEYMVYIFGVKGGLNSLPLSAIFTT |
| 64 | MLPAPKNLVVSRVTEDSARLSWTAPDAAFDSFYIVYAEPIPNGEAIVLTVPGSER |
| | SYDLTGLKPGTEYSVLIHGVKGGRNSDPLSAIFTT |
| 65 | MLPAPKNLVVSRVTKDSARLSWTAPDAAFDSFPIAYAEPRPDGEAIVLTVPGSE |
| | RSYDLTGLKPGTEYSVLIHGVKGGLLSSPLSAIFTT |
| 66 | MLPAPKNLVVSRVTEDSARLSWTAPDAAFDSFTIDYDEPRSPGEAIVLTVPGSER |
| | SYDLTGLKPGTEYRVFIWGIKGGDTSFPLSAIFTT |
| 67 | MLPAPKNLVVSRVTEDSARLSWTAPDAAFDSFTILYAEQAQFGEAIVLTVPGSE |
| | RSYDLTGLKPGTEYPITGVKGGTRSGPLSAISTT |
| 68 | MLPAPKNLVVSRVTEDSARLSWTAPDAAFDSFIIPYAEVRPDGEAIVLTVPGSER |
| | SYDLTGLKPGTEYSVLIHGVKGGHLSDPLSAISTT |
| 69 | MLPAPKNLVVSRVTEDSARLSWTAPDAAFDSFHIAYEETATSGEAIYLRVPGSE |
| | RSYDLTGLKPGTEYGVEIEGVKGGARSRPLYADFTT |
| 70 | MLPAPKNLVVSRVTEDSARLSWQGVARAFDSFLITYREQIFAGEVIVLTVPGSER |
| | SYDLTGLKPGTEYPVWIQGVKGGDLSNPLSAIFTT |
| 71 | MLPAPKNLVVSRVTEDSARLSWTAPDAAFDSFPISYLELSLYGEAIVLTVPGSER |
| | SYDLTGLKPGTEYPVGIAGVKGGVVSRPLSAIFTT |
| 72 | MLPAPKNLVVSRVTEDSARLSWTAPDAAFDSFTIGYREWYWYGEAIVLTVPGS |
| | ERSYDLTGLKPGTEYNVPISGVKGGLDSFPLSAIFTT |
| 73 | MLPAPKNLVVSEVTEDSARLSWQGVARAFDSFLITYREQIFAGEVIVLTVPGSER |
| | SYDLTGLKPGTEYPVWIQGVKGGSPSAPLSAESTT |
| 74 | MLPAPKNLVVSRVTEDSARLSWTAPDAAFDSFSITYLEWWNLGEAIVLTVPGSE |
| | RSYDLTGLKPGTEYMVTIPGVKGGMSSYPLSAIFTT |
| 75 | MLPAPKNLVVSRVTEDSARLSWTAPDAAFDSFTISYGEEALIGEAIYLRVPGSER |
| | SYDLTGLKPGTEYYVHIEGVKGGSWSQPLAAAFTT |
| 76 | MLPAPKNLVVSRVTEDSARLSWTAPDAAFDSFTIEYYENIGIGEAIVLTVPGSER |
| | SYDLTGLKPGTEYSVPIVGVKGGPYSHPLSAIFTT |
| 77 | MLPAPKNLVVSRVTEDSARLSWTAPDAAFDSFPIAYAEPRPDGEAIVLTVPGSE |
| | RSYDLTGLKPGTEYSVLIHGVKGGLLSSPLSAIFTT |
| 78 | MLPAPKNLVVSRVTEDSARLSWTAPDAAFDSFGIGYYEHKRFGEAIQLSVPGSE |
| | RSYDLTGLKPGTEYEVDIEGVKGGVLSWPLFAEFTT |
| 79 | MLPAPKNLVVSRVTEDSARLSWTAPDAAFDSFVIEYTERFWSGEAIVLTVPGSE |
| | RSYDLTGLKPGTEYSVPIDGVKGGQCSTPLSAIFTT |
| 80 | MLPAPKNLVVSRVTEDSARLSWTAPDAAFDSFWIDYEEEGVIGEAIYLHVPGSE |
| | RSYDLTGLKPGTEYVVKIHGVKGGHPSHPLVAVFTT |
| 81 | MLPAPKNLVVSRVTEDSARLSWQGVARAFDSFLITYVELRHLGEAIVLTVPGSE |
| | RSYDLTGLKPGTEYPVWIQGVKGGSPSAPLSAEFTT |
| 82 | MLPAPKNLVVSRVTEDSARLSWTAPDAAFDSFLIPYAETSPSGEAIVLTVPGSER |
| | SYDLTGLKPGTEYSVLIHGVKGGDYSSPLSAIFTT |
| 83 | MLPAPKNLVVSRVTEDSARLSWTAPDAAFDSFPIAYAEPRPDGEAIVLTVPGSE |
| | RSYDLTGLKPGTEYSVLIHGVKGGLLSSPLSAISTT |
| 84 | MLPAPKNLVVSRVTEDSARLSWQGVARAFDSFSILYLELTPKGEAIVLTVPGSE |
| | RSYDLTGLKPGTEYPVWIQGVKGGSPSAPLSAEFTT |
| 85 | MLPAPKNLVVSRVTEDSARLSWTAPDAAFDSFIIEYFEPIPIGEAIVLTVPGSERS |
| | YDLTGLKPGTEYAVNIYGVKGGYLSHPLSAIFTT |
| 86 | MLPAPKNLVVSEVTEDSARLSWQGVARAFDSFLITYREQIFAGEVIVLTVPGSEC |
| | SYDLTGLKPGTEYPVWIQGVKGGSPSAPLSAEFTT |
| 87 | MLPAPKNLVVSRVTEDSARLSWTAPDAAFDSFAIEYTEFLYSGEAIVLTVPGSER |
| | SYDLTGLKPGTEYGVPINGVKGGFVSPPLSAIVTT |
| 88 | MLPAPKNLVVSRVTEDSARLSWTAPDAAFDSFPIKYREVLRCGEAIVLTVPGSE |
| | RSYDLTGLKPGTEYTVPITGVKGGFGSSPLSAIFTT |
| 89 | MLPAPENLVVSRVTEDSARLSWTAPDAAFDSFWIEYYEGVIQGEAIVLTVPGSE |
| | RSYDLTGLKPGTEYFVAIWGVKGGKWSVPLSAIFTT |
| 90 | MLPAPKNLVVSRVTEDSARLSWQGVARAFDSFLITYREQIFAGEVIVLTVPGSER |
| | SYDLTGLKPGTEYPVWIQGVKGGSPSAEFTT |
| 91 | MLPAPKNLVVSRVTEDSARLSWTAPDAAFDSFQIHYWETQGFGEAIVLTVPGSE |
| | RSYDLTGLKPGTEYPVLIPGVKGGPSSLPLSAIFTT |
| 92 | MLPAPKNLVVSRVTEDSARLSWTAPDAAFDSFFIPYAEPSPTGEAIVLTVPGSER |
| | SYDLTGLKPGTEYSVLIHGVKGGHLSDPLSAIFTT |
| 93 | MLPAPKNLVVSRVTEDSARLSWTAPDAAFDSFAIEYYEPVPAGEAIYLDVPGSE |
| | RSYDLTGLKPGTEYDVTIYGVKGGYYSHPLFASFTT |
| 94 | MLPAPKNLVVSEVTEDSARLSWQGVARAFDSFLITYREQIFAGEVIVLTVPGSER |
| | SYDLTGLKPGTEYPVWIQGVKGGSPSAPLSAIFTT |
| 95 | MLPAPKNLVVSEVTEDSARLSWQGVARAFDSFLITYREQIFAGEVIVLTVPGSER |
| | SYDLTGLKPGTEYPVWIQGVKGGSPSAPLSAEFTT |
| 96 | MSLPAPKNLVVSRVTEDSARLSWTAPDAAFDSFPIAYLEVFYEGEAIVLTVPGSE |
| | RSYDLTGLKPGTEYQVPIEGVKGGAMSLPLSAIFTT |
| 97 | MLPAPKNLVVSRVTEDSARLSWTAPDAAFDSFHIWYEEETTIGEAIYLHVPGSE |
| | RSYDLTGLKPGTEYEVHITGVKGGPYSRPLFANFTT |
| 98 | MLPAPKNLVVSRVTEDSARLSWTAPDAAFDSFGIAYDEWPEFGEAIVLTVPGSE |
| | RSYDLTGLKPDTEYIVEIYGVKGGWFSWPLSAIFTT |
| 99 | MLPAPKNLVVSRVTEDSARLSWTAPDAAFDSFFIPYAEPSPTGEAIVLTVPGSER |
| | SYDLTGLKPGTEYSVLIHGVKGGHLSDPLSVIFTT |
| 100 | MLPAPKNLVVSRVTEDSARLSWTAPDAAFDSFYIWYEEVMYLGEAIVLTVPGS |
| | ERSYDLTGLKPGTEYNVPIPGVKGGHSSPPLSAIFTT |
| 101 | MLPAPKNLVVSRVTEDSARLSWTAPDAAFDSFHILYEELFLVGEAIVLTVPGSER |
| | SYDLTGLKPGTEYKVPISGVKGGPVSRPLSAIFTT |
| 102 | MLPAPKNLVVSRVTEDSARLSWQGVARAFDSFLITYREQIFAGEVIVLTVPGSER |
| | SYDLTGLKPGTEYPVWIQGVKGGSPSAPLSAEFTT |

Sequences of Hits from the Affinity Maturation Panning

| SEQ | |
|---|---|
| ID | Amino Acid sequence of FN3 domains that bind to CD71 |
| 103 | MLPAPKNLVVSRVTEDSARLSWTAPDAAFDSFHIVYHEPRPSGEAIWLHVPGSER |
| | SYDLTGLKPGTEYEVGIVSVKGGDLSVPLVAFFTT |
| 104 | MLPAPKNLVVSRVTEDSARLSWTAPDAAFDSFYIAYAEPRPDGEAISLLVPGSERS |
| | YDLTGLKPGTEYSVLIHGVKGGLLSSPLYAVFTT |
| 105 | MLPAPKNLVVSRVTEDSARLSWTAPDAAFDSFYIAYAEPRPDGEAIFLVVPGSERS |
| | YDLTGLKPGTEYSVLIHGVKGGLLSSPLHANFTT |
| 106 | MLPAPKNLVVSRVTEDSARLSWTAPDAAFDSFYIAYAEPRPDGEAILLDVPGSER |
| | SYDLTGLKPGTEYSVLIHGVKGGLLSSPLYASFTT |
| 107 | MSLPAPKNLVVSRVTEDSARLSWTAPDAAFDSFYIAYAEPRPDGEAISLYVPGSER |
| | SYDLTGLKPGTEYSVLIHGVKGGLLSSPLSAISTT |
| 108 | MLPAPKNLVVSRVTEDSARLSWTAPDAAFDSFFIGYLEPQPPGEAIQLRVPGSERS |
| | YDLTGLKPGTEYNVTIQGVKGGFPSMPLSAIFTT |
| 109 | MLPAPKNLVVSRVTEDSARLSWTAPDAAFDSFHISYEEDYTFGEAIYLRVPGSERS |
| | YDLTGLKPGTEYRVVIGGVKGGWFSEPLLAAFTT |
| 110 | MLPAPKNLVVSRVTEDSARLSWTAPDAAFDSFFIGYLEPQPPGEAIYLTVPGSERS |
| | YDLTGLKPGTEYNVTIQGVKGGFPSYPLDASFTT |
| 111 | MLPAPKNLVVSRVTEDSARLSWTAPDAAFDSFFIGYLEPQPPGEAIDLGVPGSERS |
| | YDLTGLKPGTEYNVTIQGVKGGFPSMPLDPLEAYFTT |
| 112 | MLPAPKNLVVSRVTEDSARLSWTAPDAAFDSFYIAYAEPRPDGEAILLLVPGSERS |
| | YDLTGLKPGTEYSVLIHGVKGGLLSSPLSAIFTT |
| 113 | MLPAPKNLVVSRVTEDSARLSWTAPDAAFDSFYIAYAEPRPDGEAINLQVPGSER |
| | SYDLTGLKPGTEYSVLIHGVKGGLLSSPLVAFFTT |
| 114 | MLPAPKNLVVSRVTEDSARLSWTTPDAAFDSFFIGYLEPQPPGEAISLQVPGSERS |
| | YDLTGLKPGTEYNVTIQGVKGGFPSSPLFAVFTT |
| 115 | MLPAPKNLVVSRVTEDSARLSWTAPDAAFDSFYIAYAEPRPDGEAIELHVPGSER |
| | SYDLTGLKPGTEYSVLIHGVKGGLLSSPLFTT |
| 116 | MLPAPKNLVVSRVTEDSARLSWTAPDAAFDSFYIAYAEPRPDGEAIQLVVPGSER |
| | SYDLTGLKPGTEYSVLIHGVKGGLLSSPLSAIFTT |
| 117 | MLPAPKNLVVSRVTEDSARLSWTAPDAAFDSFYIAYAEPRPDGEAITLDVPGSER |
| | SYDLTGLKPGTEYSVLIHGVKGGLLSSPLSAIFTT |
| 118 | MLPAPKNLVVSRVTEDSARLSWTAPDAAFDSFYIAYAEPRPDGEAIWLVVPGSER |
| | SYDLTGLKPGTEYSVLIHGVKGGLLSSPLVASFTT |
| 119 | MLPAPKNLVVSRVTEDSARLSWTAPDAAFDSFYIAYAEPRPDGEAINLDVPGSER |
| | SYDLTGLKPGTEYSVLIHGVKGGLLSSPLVAEFTT |
| 120 | MLPAPKNLVVSRVTEDSARLSWTAPDAAFDSFYIAYAEPRPDGEAIHLSVPGSERS |
| | YDLTGLKPGTEYSVLIHGVKGGLLSSPLVAIFTT |
| 121 | MLPAPKNLVVSRVTEDSARLSWTAPDAAFDSFFIGYLEPQPPGEAIALWVPGSERS |
| | YDLTGLKPGTEYNVTIQGVKGGFPSMPLSAIFTT |
| 122 | MLPAPKNLVVSRVTEDSARLSWTAPDAAFDSFYIAYAEPRPDGEAIILVVPGSERS |
| | YDLTGLKPGTEYSVLIHGVKGGLLSSPLSAHFTT |
| 123 | MLPAPKNLVVSRVTEDSARLSWTAPDAAFDSFFIGYLEPQPPGEAIQLWVPGSERS |
| | YDLTGLKPGTEYNVTIQGVKGGFPSHPLGAVFTT |
| 124 | MLPAPKNLVVSRVTEDSARLSWTAPDAAFDSFYIAYAEPRPDGEAIQLHVPGSER |
| | SYDLTGLKPGTEYSVLIHGVKGGLLSSPLLASFTT |
| 125 | MLPAPKNLVVSRVTEDSARLSWTAPDAAFDSFYIAYAEPRPDGEAIALHVPGSER |
| | SYDLTGLKPGTEYSVLIHGVKGGLLSSPLVAFFTT |
| 126 | MLPAPKNLVVSRVTEDSARLSWTAPDAAFDSFFIGYLEPQPPGEAIHLHVPGSERS |
| | YDLTGLKPGTEYNVTIQGVKGGFPSIPLHANFTT |
| 127 | MLPAPKNLVVSRVTEDSARLSWTAPDAAFDSFYIAYAEPRPDGEAIFLGVPGSERS |
| | YDLTGLKPGTEYSVLIHGVKGGLLSSPLSAIFTT |
| 128 | MLPAPKNLVVSRVTEDSARLSWTAPDAAFDSFYIAYAEPRPDGEAIVLRVPGSER |
| | SYDLTGLKPGTEYSVLIHGVKGGLLSSPLIASFTT |
| 129 | MLPAPKNLVVSRVTEDSARLSWTAPDAAFDSFYIAYAEPRPDGEAINLWVPGSER |
| | SYDLTGLKPGTEYSVLIHGVKGGLLSSPLDASFTT |
| 130 | MLPAPKNLVVSRVTEDSARLSWTAPDAAFDSFVIEYFEWTLNGEAIVLTVPGSER |
| | SYDLTGLKPGTEYSVQIYGVKGGCLSRPLSAIFTT |
| 131 | MLPAPKNLVVSRVTEDSARLSWTAPDAAFDSFYIAYAEPRPDGEAIHLWVPGSER |
| | SYDLTGLKPGTEYSVLIHGVKGGLLSSPLIAHFTT |
| 132 | MLPAPKNLVVSRVTEDSARLSWTAPDAAFDSFFIPYAEPSPTGEAIVLTVPGSERS |
| | YDLTGLKPGTEYSVLIHGVKGGLLSSPLSAHFTT |
| 133 | MLPAPKNLVVSRVTEDSARLSWTAPDAAFDSFYIAYAEPRPDGEAIYLYVPGSER |
| | SYDLTGLKPGTEYSVLIHGVKGGLLSSPLDAFFTT |
| 134 | MLPAPKNLVVSRVTEDSARLSWTAPDAAFDSFYIAYAEPRPDGEAIGLQVPGSER |
| | SYDLTGLKPGTEYSVLIHGVKGGLLSSPLSAIFTT |
| 135 | MLPAPKNLVVSRVTEDSARLSWTAPDAAFDSFYIAYAEPRPDGEAIQLAVPGSER |
| | SYDLTGLKPGTEYSVLIHGVKGGLLSSPLHAFFTT |
| 136 | MLPAPKNLVVSRVTEDSARLSWTAPDAAFDSFFIGYLEPQPPGEAIWLHVPGSERS |
| | YDLTGLKPGTEYNVTIQGVKGGFPSIPLIAIFTT |
| 137 | MLPAPKNLVVSRVTEDSARLSWTAPDAAFDSFYIAYAEPRPDGEAIVLDVPGSER |
| | SYDLTGLKPGTEYSVLIHGVKGGLLSSPLVAEFTT |
| 138 | MLPTPKNLVVSRVTEDSARLSWTAPDAAFDSFYIAYAEPRPDGEAIQLRVPGSERS |
| | YDLTGLKPGTEYSVLIHGVKGGLLSSPLHASFTT |
| 139 | MLPAPKNLVVSRVTEDSARLSWTAPDAAFDSFFIGYLEPQPPGEAIQLGVPGSERS |
| | YDLTGLKPGTEYNVTIQGVKGGFPSHPLNANFTT |
| 140 | MLPAPKNLVVSRVTEDSARLSWTAPDAAFDSFFIGYLEPQPPGEAIQLEVPGSERS |
| | YDLTGLKPGTEYNVTIQGVKGGFPSMPLSAIFTT |
| 141 | MLPAPKNLVVSRVTEDSARLSWTAPDAAFDSFYIAYAEPRPDGEAIFLGVPGSERS |
| | YDLTGLKPGTEYSVLIHGVKGGLLSSPLIAFFTT |
| 142 | MLPAPKNLVVSRVTEDSARLSWTAPDAAFDSFFIGYLEPQPPGEAIGLQVPGSERS |
| | YDLTGLKPGTEYNVTIQGVKGGFPSHPLKAQFTT |
| 143 | MLPAPKNLVVSRVTEDSARLSWTAPDAAFDSFYIAYAEPRPDGEAILLFVPGSERS |
| | YDLTGLKPGTEYSVLIHGVKGGLLSSPLVAHFTT |
| 144 | MLPAPKNLVVSRVTEDSARLSWTAPDAAFDSFYIAYAEPRPDGEAIGLYVPGSER |
| | SYDLTGLKPGTEYSVLIHGVKGGLLSSPLGAFFTT |
| 145 | MLPAPKNLVVSRVTEDSARLSWTAPDAAFDSFYIAYAEPRPDGEAILLQVPGSER |
| | SYDLTGLKPGTEYSVLIHGVKGGLLSSPLTAIFTT |
| 146 | MLPAPKNLVVSRVTEDSARLSWTAPDAAFDSFYIAYAEPRPDGEAITLHVPGSER |
| | SYDLTGLKPGTEYSVLIHGVKGGLLSSPLSAIFTT |
| 147 | MLPAPKNLVVSRVTEDSARLSWTAPDAAFDSFYIAYAEPRPDGEAIVLEVPGSER |
| | SYDLTGLKPGTEYSVLIHGVKGGLLSSPLDAHFTT |
| 148 | MLPAPKNLVVSRVTEDSARLSWTAPDAAFDSFYIAYAEPRPDGEAIALHVPGSER |
| | SYDLTGLKPGTEYSVLIHGVKGGLLSSPLRAVFTT |
| 149 | MLPAPKNLVVSRVTEDSARLSWTAPDAAFDSFYIAYAEPRPDGEAIQLWVPGSER |
| | SYDLTGLKPGTEYSVLIHGVKGGLLSSPLSAIFTT |
| 150 | MLPAPKNLVVSRVTEDSARLSRTAPDAAFDSFYIAYAEPRPDGEAIVLIVPGSERS |
| | YDLTGLKPGTEYSVLIHGVKGGLLSSPLSAIFTT |
| 151 | MLPAPKNLVVSRVTEDSARLSWTAPDAAFDSFFIGYLEPQPPGEAIQLWVPGSERS |
| | YDLTGLKPGTEYNVTIQGVKGGFPSRPLQAHFTT |
| 152 | MLPAPKNLVVSRVTEDSARLSWTAPDAAFDSFYIAYAEPRPDGEAITLDVPGSER |
| | SYDLTGLKPGTEYSVLIHGVKGGLLSSPLYAFFTT |
| 153 | MLPAPKNLVVSRVTEDSARLSWTAPDAAFDSFYIAYAEPRPDGEAIALHVPGSER |
| | SYDLTGLKPGTEYSVLIHGVKGGLLSSPLSAIFTT |
| 154 | MLPAPKNLVVSRVTEDSARLSWTAPDAAFDSFYIAYAEPRPDGEAIGLWVPGSER |
| | SYDLTGLKPGTEYSVLIHGVKGGLLSSPLIAHFTT |
| 155 | MLPAPKNLVVSRVTEDSARLSWTAPDAAFDSFYIAYAEPRPDGEAIWLVVPGSER |
| | SYDLTGLKPGTEYSVLIHGVKGGLLSSPLHARFTT |
| 156 | MLPAPKNLVVSRVTEDSARLSWTAPDAAFDSFYIAYAEPRPDGEAIFLQVPGSERS |
| | YDLTGLKPGTEYSVLIHGVKGGLLSSPLAAVFTT |
| 157 | MLPAPKNLVVSRVTEDSARLSWTAPDAAFDSFYIAYAEPRPDGEAILLHVPGSER |
| | SYDLTGLKPGTEYSVLIHGVKGGLLSSPLSAISTT |
| 158 | MLPAPKNLVVSRVTEDSARLSWTAPDAAFDSFYIAYAEPRPDGEAIILQVPGSERS |
| | YDLTGLKPGTEYSVLIHGVKGGLLSSPLSAVFTT |
| 159 | MLPAPKNLVVSRVTEDSARLSWTAPDAAFDSFYIAYAEPRPDGEAIYLKVPGSER |
| | SYDLTGLKPGTEYSVLIHGVKGGLLSSPLVAHFTT |
| 160 | MLPAPKNLVVSRVTEDSARLSWTAPDAAFDSFYIAYAEPRPDGEAIVLTVPGSER |
| | SYDLTGLKPGTEYSVLIHGVKGGLLSSPLLAYFTT |
| 161 | MLPAPKNLVVSRVTEDSARLSWTAPDAAFDSFYIAYAEPRPDGEAIILHVPGSERS |
| | YDLTGLKPGTEYSVLIHGVKGGLLSSPLEAKFTT |
| 162 | MLPAPKNLVVSRVTEDSARLSWTAPDAAFDSFYIAYAEPRPDGEAIKLEVPGSER |
| | SYDLTGLKPGTEYSVLIHGVKGGLLSSPLAIFTT |
| 163 | MLPAPKNLVVSRVTEDSARLSWTAPDAAFDSFFIGYLEPQPPGEAIYLEVPGSERS |
| | YDLTGLKPGTEYNVTIQGVKGGFPSFPLKAAFTT |
| 164 | MLPAPKNLVVSRVTEDSARLSWTAPDAAFDSFYIAYAEPRPDGEAIILRVPGSERS |
| | YDLTGLKPGTEYSVLIHGVKGGLLSSPLVAIFTT |
| 165 | MLPAPKNLVVSRVTEDSARLSWTAPDAAFDSFYIAYAEPRPDGEAIVLQVPGSER |
| | SYDLTGLKPGTEYSVLIHGVKGGLLSSPLAAWFTT |
| 166 | MLPAPKNLVVSRVTEDSARLSWTAPDAAFDSFYIAYAEPRPDGEAIFLQVPGSERS |
| | YDLTGLKPGTEYSVLIHGVKGGLLSSPLNAFFTT |
| 167 | MLPAPKNLVVSRVTEDSARLSWTAPDAAFDSFFIGYLEPQPPGEAIILGVPGSERS |
| | YDLTGLKPGTEYSVLIHGVKGGLLSSPLHAYSTT |
| 168 | MLPAPKNLVVSRVTEDSARLSWTAPDAAFDSFYIAYAEPRPDGEAILLDVPGSER |
| | SYDLTGLKPGTEYSVLIHGVKGGLLSSPLSAIFTT |
| 169 | MLPAPKNLVVSRVTEDSARLSWTAPDAAFDSFYIAYAEPRPDGEAILLLVPGSERS |
| | YDLTGLKPGTEYSVLIHGVKGGLLSSPLSAVFTT |
| 170 | MLPAPKNLVVSRVTEDSARLSWTAPDAAFDSFYIAYAEPRPDGEAIHLLVPGSER |
| | SYDLTGLKPGTEYSVLIHGVKGGLLSSPLLAHFTT |
| 171 | MLPAPKNLVVSRVTEDSARLSWTAPDAAFDSFYIAYAEPRPDGEAIQLWVPGSER |
| | SYDLTGLKPGTEYSVLIHGVKGGLLSSPLDAYFTT |
| 172 | MLPAPKNLVVSRVTEDSARLSWTAPDAAFDSFFIGYLEPQPPGEAIVLTVPGSERS |
| | YDLTGLKPGTEYNVTIQGVKGGFPSMPLASFTT |
| 173 | MLPAPKNLVVSRVTEDSARLSWTAPDAAFDSFHIVYHEPRPSGEAIHLQVPGSERS |
| | YDLTGLKPGTEYNVTIQGVKGGFPSYPLSAFFTT |
| 174 | MLPAPKNLVVSRVTEDSARLSWTAPDAAFDSFFIGYLEPQPPGEAIQLWVPGSERS |
| | YDLTGLKPGTEYNVTIQGVKGGFPSMPLSAIFTT |
| 175 | MLPAPKNLVVSRVTEDSARLSWTAPDAAFDSFRISYCETFYHGEAIVLTVPGSERS |
| | YDLTGLKPGTEYSVLIHGVKGGLLSSPLIAKFTT |
| 176 | MLPAPKNLVVSRVTEDSARLSWTAPDAAFDSFYIAYAEPRPDGEAIWLKVPGSER |
| | SYDLTGLKPGTEYSVLIHGVKGGLLSSPLQANFTT |
| 177 | MLPAPKNLVVSRVTEDSARLSWTAPDAAFDSFYIAYAEPRPDGEAIWLKVPGSER |
| | SYDLTGLKPGTEYSVLIHGVKGGLLSSPLQANFTT |
| 178 | MLPAPKNLVVSRVTEDSARLSWTAPDAAFDSFYIAYAEPRPDGEAIQLQVPGSER |
| | SYDLTGLKPGTEYSVLIHGVKGGLLSSPLSAIVTT |
| 179 | MLPAPKNLVVSRVTEDSARLSWTAPDAAFDSFPIAYAEPRPDGEAIVLTVPGSERS |
| | YDLTGLKPGTEYSVLIHGVKGGLLSSPLSAFFTT |
| 180 | MLPAPKNLVVSRVTEDSARLSWTAPDAAFDSFYIAYAEPRPDGEAIALLVPGSER |
| | SYDLTGLKPGTEYSVLIHGVKGGLLSSPLVAQFTT |
| 181 | MLPAPKNLVVSRVTEDSARLSWTAPDAAFDSFYIAYAEPRPDGEAIILHVPGSERS |
| | YDLTGLKPGTEYSVLIHGVKGGLLSSPLEAKFTT |
| 182 | MLPAPKNLVVSRVTEDSARLSWTAPDAAFDSFYIAYAEPRPDGEAIDLHVPGSER |
| | SYDLTGLKPGTEYSVLIHGVKGGLLSSPLHALFTT |
| 183 | MLPAPKNLVVSRVTEDSARLSWTAPDAAFDSFYIAYAEPRPDGEAILLDVPGSER |
| | SYDLTGLKPGTEYSVLIHGVKGGFPSMPLSAIFTT |
| 184 | MLPAPKNLVVSRVTEDSARLSWTAPDAAFDSFFIGYLEPQPPGEAIDLAVPGSERS |
| | YDLTGLKPGTEYSVLIHGVKGGLLSSPLSFTT |
| 185 | MLPAPKNLVVSRVTEDSARLSWTAPDAAFDSFYIAYAEPRPDGEAIYLGVPGSER |
| | SYDLTGLKPGTEYSVLIHGVKGGLLSSPLRAKFTT |
| 186 | MLPAPKNLVVSRVTEDSARLSWTAPDAAFDSFYIAYAEPRPDGEAIQLGVPGSER |
| | SYDLTGLKPGTEYSVLIHGVKGGLLSSPLSAIFTT |
| 187 | MLPAPKNLVVSRVTEDSARLSWTAPDAAFDSFFIGYLEPQPPGEAISLLVPDSERS |
| | YDLTGLKPGTEYNVTIQGVKGGFPSMPLKFTT |
| 188 | MLPAPKNLVVSRVTEDSARLSWTAPDAAFDSFYIAYAEPRPDGEAIGLGVPGSER |
| | SYDLTGLKPGTEYSVLIHGVKGGLLSSPLDASFTT |
| 189 | MLPAPKNLVVSRVTEDSARLSWTAPDAAFDSFYIAYAEPRPDGEAIQLTVPGSER |
| | SYDLTGPKPGTEYWVLIQGVKGGGSSVPLVAYFTT |
| 190 | MLPAPKNLVVSRVTEDSARLSWTAPDAAFDSFYIAYAEPRPDGEAISLDVPGSERS |
| | YDLTGLKPGTEYSVLIHGVKGGLLSSPLEASFTT |
| 191 | MLPAPKNLVVSRVTEDSARLSWTAPDAAFDSFFIGYLEPQPPGEAIILAVPGSERS |
| | YDLTGLKPGTEYNVTIQGVKGGFPSLPLVASFTT |
| 192 | MLPAPKNLVVSRVTEDSARLSWTAPDAAFDSFDIGYTEYGGYGEAIYLSVPGSER |
| | SYDLTGLKPGTEYWVLIQGVKGGGSSVPLSAIFTT |
| 193 | MLPAPKNLVVSRVTEDSARLSWTAPDAAFDSFYIAYAEPRPDGEAISLSVPGSERS |
| | YDLTGLKPGTEYSVLIHGVKGGLLSSPLIANFTT |
| 194 | MLPAPKNLVVSRVTEDSARLSWTAPDAAFDSFYIAYAEPRPDGEAIALLVPGSER |
| | SYDLTGLKPGTEYSVLIHGVKGGLLSSPLSAIVTT |
| 195 | MLPAPKNLVVSRVTEDSARLSWTAPDAAFDSFYIAYAEPRPDGEAIILDVPGSERS |
| | YDLTGLKPGTEYSVLIHGVKGGLLSSPLSSIFTT |
| 196 | MLPAPKNLVVSRVTEDSARLSWTAPDAAFDSFYIAYAEPRPDGEAIVLWVPGSER |
| | SYDLTGLKPGTEYSVLIHGVKGGLLSSPLRASFTT |
| 197 | MLPAPKNLVVSRVTEDSARLSWTAPDAAFDSFYIAYAEPRPDGEAIKLDVPGSER |
| | SYDLTGLKPGTEYSVLIHGVKGGLLSSPLVAFFTT |
| 198 | MLPAPKNLVVSRVTEDSARLSWTAPDAAFDSFYIAYAEPRPDGEAIILEVPGSERS |
| | YDLTGLKPGTEYSVLIHGVKGGLLSSPLVAYFTT |
| 199 | MLPAPKNLVVSRVTEDSARLSWTAPDAAFDSFYIAYAEPRPDGEAIHLWVPGSER |
| | SYDLTGLKPGTEYSVLIHGVKGGLLSSPLHADFTT |
| 200 | MLPAPKNLVVSRVTEDSARLSWTAPDAAFDSFYIAYAEPRPDGEAIWLEVPGSER |
| | SYDLTGLKPGTEYSVLIHGVKGGLLSSPLVADFTT |
| 201 | MLPAPKNLVVSRVTEDSARLSWTAPDAAFDSFYIAYAEPRPDGEAISLWVPGSER |
| | SYDLTGLKPGTEYSVLIHGVKGGLLSSPLLAHFTT |
| 202 | MLPAPKNLVVSRVTEDSARLSWTAPDAAFDSFDIGYTEYGGYGEAILHVPGSERS |
| | YDLTGLKPGTEYWVLIQGVKGGGSSVPLSAIFTT |
| 203 | MLPAPKNLVVSRVTEDSARLSWTAPDAAFDSFFIGYLEPQPPGEAIVLLVPGSERS |
| | YDLTGLKPGTEYNVTIQGVKGGFPSVPLAAFFTT |
| 204 | MLPAPKNLVVSRVTEDSARLSWTAPDAAFDSFFIGYLEPQPPGEAILLWVPGSERS |
| | YDLTGLKPGTEYNVTIQGVKGGFPSQFTT |
| 205 | MLPAPKNLVVSRVTEDSARLSWTAPDAAFDSFFIGYLEPQPPGEAILLGVPGSERS |
| | YDLTGLKPGTEYNVTIQGVKGGFPSMPLHPLVALFTT |
| 206 | MLPAPKNLVVSRVTEDSARLSWTAPDAAFDSFYIAYAEPRPDGEAIGLDVPGSER |
| | SYDLTGLKPGTEYSVLIHGVKGGLLSSPLSAIFTT |
| 207 | MLPAPKNLVVSRVTEDSARLSWTAPDAAFDSFYIAYAEPRPDGEAIHLSVPGSERS |
| | YDLTGLKPGTEYSVLIHGVKGGLLSSPLAAYFTT |
| 208 | MLPAPKNLVVSRVTEDSARLSWTAPDAAFDSFFIGYLEPQPPGEAIVLAVPGSERS |
| | YDLTGLKPGTEYNVTIQGVKGGFPSYPLVAAFTT |
| 209 | MLPAPKNLVVSRVTEDSARLSWTAPDAAFDSFYIAYAEPRPDGEAILLQVPGSCR |
| | SYDLTGLKPGTEYSVLIHGVKGGLLSSPLTAIFTT |
| 210 | MLPAPKNLVVSRVTEDSARLSWTAPDAAFDSFFIGYLEPQPPGEAINLQVPGSERS |
| | YDLTGLKPGTEYNVTIQGVKGGFPSFPLSAVFTT |
| 211 | MLPAPKNLVVSRVTEDSARLSWTAPDAAFDSFYIAYAEPRPDGEAIQLHVPGSER |
| | SYDLTGLKPGTEYSVLIHGVKGGLLSSPLVAIFTT |
| 212 | MLPAPKNLVVSRVTEDSARLSWTAPDAAFDSFYIAYAEPRPDGEAIWLAVPGSER |
| | SYDLTGLKPGTEYSVLIHGVKGGLLSSPLHAQFTT |
| 213 | MLPAPKNLVVSRVTEDSARLSWTAPDAAFDSFYIAYAEPRPDGEAILLGVPGSER |
| | SYDLTGLKPGTEYSVLIHGVKGGLLSSPLFTT |
| 214 | MLPAPKNLVVSRVTEDSARLSWTAPDAAFDSFYIAYAEPRPDGEAIGLQVPGSER |
| | SYDLTGLKPGTEYSVLIHGVKGGLLSSPLCAEFTT |
| 215 | MLPAPKNLVVSRVTEDSARLSWTAPDAAFDSFYIAYAEPRPDGEAIVLWVPGSER |
| | SYDLTGLKPGTEYSVLIHGVKGGLLSSPLIAEFTT |
| 216 | MLPAPKNLVVSRVTEDSARLSWTAPDAAFDSFYIAYAEPRPDGEAISLSVPGSERS |
| | YDLTGLKPGTEYSVLIHGVKGGLLSSPPKFTT |
| 217 | MLPAPKNLVVSRVTEDSARLSWTAPDAAFDSFYIAYAEPRPDGEAIILEVPGSERS |
| | YDLTGLKPGTEYSVLIHGVKGGLLSSPLRAVFTT |
| 218 | MLPAPKNLVVSRVTEDSARLSWTAPDAAFDSFYIAYAEPRPDGEAIHLVVPGSER |
| | SYDLTGLKPGTEYSVLIHGVKGGLLSSPLSAIFTT |
| 219 | MLPAPKNLVVSRVTEDSARLSWTAPDAAFDSFYIAYAEPRPDGEAISLKVPGSERS |
| | YDLTGLKPGTEYSVLIHGVKGGLLSSPLEAIFTT |
| 220 | MLPAPKNPVVSRVTEDSARLSWTAPDAAFDSFYIAYAEPRPDGEAIHLLVPGSERS |
| | YDLTGLKPGTEYSVLIHGVKGGLLSSPLKRLSPPVVTITITMAVCRKPVAENLSQT |
| | LS |
| 221 | MLPAPKNLVVSRVTEDSARLSWTAPDAAFDSFFIGYLEPQPPGEAIFLDVPGSERS |
| | YDLTGLKPGTEYNVTIQGVKGGFPSPLTAFFTT |
| 222 | MLPAPKNLVVSRVTEDSARLSWTAPDAAFDSFFIGYLEPQPPGEAIVLDVPGSERS |
| | YDLTGLKPGTEYNVTIQGVKGGFPSHPLAAAFTT |
| 223 | MLPAPKNLVVSRVTEDSARLSWTAPDAAFDSFFIGYLEPQPPGEAIGLAVPGSERS |
| | YDLTGLKPGTEYNVTIQGVKGGFPSVPLQANFTT |
| 224 | MLPAPKNLVVSRVTEDSARLSWTAPDAAFDSFYIAYAEPRPDGEAILLRVPGSERS |
| | YDLTGLKPGTEYSVLIHGVKGGLLSSPLVAEFTT |
| 225 | MLPAPKNLVVSRVTEDSARLSWTAPDAAFDSFYIAYAEPRPDGEAILLQVPGSER |
| | SYDLTGLKPGTEYSVLIHGVKGGLLSSPLSASFTT |
| 226 | MLPAPKNLVVSRVTEDSARLSWTAPDAAFDSFYIAYAEPRPDGEAIGLHVPGSER |
| | SYDLTGLKPGTEYSVLIHGVKGGLLSSPLTASFTT |
| 227 | MLPAPKNLVVSRVTEDSARLSWTAPDAAFDSFYIAYAEPRPDGEAIGLRVPGSER |
| | SYDLTGLKPGTEYSVLIHGVKGGLLSSPLSAIFTT |
| 228 | MLPAPKNLVVSRVTEDSARLSWTAPDAAFDSFYIAYAEPRPDGEAIVLRVPGSER |
| | SYDLTGLKPGTEYSVLIHGVKGGLLSSPLAASFTT |
| 229 | MLPAPKNLVVSRVTEDSARLSWTAPDAAFDSFYIAYAEPRPDGEAIQLLVPGSER |
| | SYDLTGLKPGTEYSVLIHGVKGGLLSSPLVAHFTT |
| 230 | MLPAPKNLVVSRVTEDSARLSWTAPDAAFDSFYIAYAEPRPDGEAIWLLVPGSER |
| | SYDLTGLKPGTEYSVLIHGVKGGLLSSPLVAFFTT |
| 231 | MLPAPKNLVVSRVTEDSARLSWTAPDAAFDSFFIGYLEPQPPGEAIHLYVPGSERS |
| | YDLTGLKPGTEYNVTIQGVKGGFPSDPLDAVFTT |
| 232 | MLPAPKNLVVSRVTEDSARLSWTAPDAAFDSFFIGYLEPQPPGEAIYLDVPGSERS |
| | YDLTGLKPGTEYNVTIQGVKGGFPSTFTT |
| 233 | MLPAPKNLVVSRVTEDSARLSWTAPDAAFDSFYIAYAEPRPDGEAIQLFVPGSERS |
| | YDLTGLKPGTEYSVLIHGVKGGLLSSPLKAYFTT |
| 234 | MLPAPKNLVVSRVTEDSARLSWTAPDAAFDSFYIAYAEPRPDGEAIVLVVPGSER |
| | SYDLTGLKPGTEYSVLIHGVKGGLLSSPLSAIFTT |
| 235 | MLPAPKNLVVSRVTEDSARLSWTAPDAAFDSFFIGYLEPQPPGEAIQLTVPGSERS |
| | YDLTGLKPGTEYNVTIQGVKGGFPSLPLSADFTT |
| 236 | MLPAPKNLVVSRVTEDSARLSWTAPDAAFDSFYIAYAEPRPDGEAILLQVPGSER |
| | SYDLTGLKPGTEYSVLIHGVKGGLLSSPLDAEFTT |
| 237 | MLPAPKNLVVSRVTEDSARLSWTAPDAAFDSFYIAYAEPRPDGEAIVLAVPGSER |
| | SYDLTGLKPGTEYSVLIHGVKGGLLSSPLYASFTT |
| 238 | MLPAPKNLVVSRVTEDSARLSWTTPDAAFDSFYIAYAEPRPDGEAIRLQVPGSERS |
| | YDLTGLKPGTEYSVLIHGVKGGLLSSPLGFTT |
| 239 | MLPAPKNLVVSRVTEDSARLSWTAPDAAFDSFYIAYAEPRPDGEAIQLVVPGSER |
| | SYDLTGLKPGTEYSVLIHGVKGGLLSSPLYAIFTT |
| 240 | MLPAPKNLVVSRVTEDSARLSWTAPDAAFDSFYIAYAEPRPDGEAISLSVPGSERS |
| | YDLTGLKPGTEYSVLIHGVKGGLLSSPLHAKFTT |
| 241 | MLPAPKNLVVSRVTEDSARLSWTAPDAAFDSFFIGYLEPQPPGEAIHLGVPGSERS |
| | YDLTGLKPGTEYNVTIQGVKGGFPSIPLFASFTT |
| 242 | MLPAPKNLVVSRVTEDSARLSWTAPDAAFDSFYIAYAEPRPDGEAIQLLVPGSER |
| | SYDLTGLKPGTEYSVLIHGVKGGLLSSPLYAAFTT |
| 243 | MLPAPKNLVVSRVTEDSARLSWTAPDAAFDSFFIGYLEPQPPGEAIHLAVPGSERS |
| | YDLTGLKPGTEYNVTIQGVKGGFPSVPLAAVFTT |
| 244 | MLPAPKNLVVSRVTEDSARLSWTAPDAAFDSFYIAYAEPRPDGEAISLQVPGSERS |
| | YDLTGLKPGTEYSVLIHGVKGGLLSSPLGAHFTT |
| 245 | MLPAPKNLVVSRVTEDSARLSWTAPDAAFDSFYIAYAEPRPDGEAIALWVPGSER |
| | SYDLTGLKPGTEYSVLIHGVKGGLLSSPLVASFTT |
| 246 | MLPAPKNLVVSRVTEDSARLSWTAPDAAFDSFYIAYAEPRPDGEAIQLHVPGSER |
| | SYDLTGLKPGTEYSVLIHGVKGGLLSSPLYAFFTT |
| 247 | MLPAPKNLVVSRVTEDSARLSWTAPDAAFDSFYIAYAEPRPDGEAILLHVPGSER |
| | SYDLTGLKPGTEYSVLIHGVKGGLLSSPLRASFTT |
| 248 | MLPAPKNLVVSRVTEDSARLSWTAPDAAFDSFYIAYAEPRPDGEAIWLGVPGSER |
| | SYDLTGLKPGTEYSVLIHGVKGGLLSSPLHATFTT |
| 249 | MLPAPKNLVVSRVTEDSARLSWTAPDAAFDSFYIAYAEPRPDGEAIVLEVPGSER |
| | SYDLTGLKPGTEYSVLIHGVKGGLLSSPLHANFTT |
| 250 | MLPAPKNLVVSRVTEDSARLSWTAPDAAFDSFYIAYAEPRPDGEAILLRVPGSERS |
| | YDLTGLKPGTEYSVLIHGVKGGLLSSPLYAKFTT |
| 251 | MLPAPKNLVVSRVTEDSARLSWTAPDAAFDSFFIGYLEPQPPGEAIGLWVPGSERS |
| | YDLTGLKPGTEYNVTIQGVKGGFPSDPLQAVFTT |
| 252 | MLPAPKNLVVSRVTEDSARLSWTAPDAAFDSFYIAYAEPRPDGEAIVLHVPGSER |
| | SYDLTGLKPGTEYSVLIHGVKGGLLSSPLDAFFTT |
| 253 | MLPAPKNLVVSRVTEDSARLSWTAPDAAFDSFYIAYAEPRPDGEAIILHVPGSERS |
| | YDLTGLKPGTEYSVLIHGVKGGLLSSPLDAYFTT |
| 254 | MLPAPKNLVVSRVTEDSARLSWTAPDAAFDSFYIAYAEPRPDGEAILLAVPGSER |
| | SYDLTGLKPGTEYSVLIHGVKGGLLSSPLSAKFTT |
| 255 | MLPAPKNLVVSRVTEDSARLSWTAPDAAFDSFFIGYLEPQPPGEAILLFVPGSERS |
| | YDLTGLKPGTEYNVTIQGVKGGFPSTPLSASFTT |
| 256 | MLPAPKNLVVSRVTEDSARLSWTAPDAAFDSFYIAYAEPRPDGEAIQLTVPGSER |
| | SYDLTGLKPGTEYSVLIHGVKGGLLSSPLHAYFTT |
| 257 | MLPAPKNLVVSRVTEDSARLSWTAPDAAFDSFYIAYAEPRPDGEAIQLGVPGSER |
| | SYDLTGLKPGTEYSVLIHGVKGGLLSSPLRAYFTT |
| 258 | MLPAPKNLVVSRVTEDSARLSWTAPDAAFDSFYIAYAEPRPDGEAILLEVPGSERS |
| | YDLTGLKPGTEYSVLIHGVKGGLLSSPLVAFFTT |
| 259 | MLPAPKNLVVSRVTEDSARLSWTAPDAAFDSYIAYAEPRPDGEAIQLGVPGSER |
| | SYDLTGLKPGTEYSVLIHGVKGGLLSSPLLAVFTT |
| 260 | MLPAPKNLVVSRVTEDSARLSWTAPDAAFDSFYIAYAEPRPDGEAIHLRVPGSER |
| | SYDLTGLKPGTEYNVTIQGVKGGFPSMPLSAIFTT |
| 261 | MLPAPKNLVVSRVTEDSARLSWTAPDAAFDSFYIAYAEPRPDGEAILLQVPGSER |
| | SYDLTGLKPGTEYSVLIHGVKGGLLSSPLIAKFTT |
| 262 | MLPAPKNLVVSRVTEDSARLSWTAPDAAFDSFYIAYAEPRPDGEAIVLHVPGSER |
| | SYDLTGLKPGTEYSVLIHGVKGGLLSSPLQAIFTT |
| 263 | MLPAPKNLVVSRVTEDSARLSWTAPDAAFDS FYIAYAEPRPDGEAIALVVPGSER |
| | SYDLTGLKPGTEYSVLIHGVKGGLLSSPLAANFTT |
| 264 | MLPAPKNLVVSRVTEDSARLSWTAPDAAFDSFYIAYAEPRPDGEAINLSVPGSERS |
| | YDLTGLKPGTEYSVLIHGVKGGLLSSPLDAYFTT |
| 265 | MLPAPKNLVVSRVTEDSARLSWTAPDAAFDSFYIAYAEPRPDGEAIVLEVPGSER |
| | SYDLTGLKPGTEYSVLIHGVKGGLLSSPLTASFTT |
| 266 | MLPAPKNLVVSRVTEDSARLSWTAPDAAFDSFYIAYAEPRPDGEAIRLQVPGSER |
| | SYDLTGLKPGTEYSVLIHGVKGGLLSSPLGASFTT |
| 267 | MLPAPKNLVVSRVTEDSARLSWTAPDAAFDSFYIAYAEPRPDGEAIGLWVPGSER |
| | SYDLTGLKPGTEYSVLIHGVKGGLLSSPLVAYFTT |
| 268 | MLPAPKNLVVSRVTEDSARLSWTAPDAAFDSFYIAYAEPRPDGEAIYLEVPGSER |
| | SYDLTGLKPGTEYSVLIHGVKGGLLSSPLFTT |
| 269 | MLPAPKNLVVSRVTEDSARLSWTAPDAAFDSFYIAYAEPRPDGEAIWLDVPGSER |
| | SYDLTGLKPGTEYSVLIHGVKGGLLSSPLDAYFTT |
| 270 | MLPAPKNLVVSRVTEDSARLSWTAPDAAFDSFAIEYCETKMCGEAIVLTVPGSER |
| | SYDLTGLKPGTEYRVPIPGVKGGTASLPLSAIFTT |
| 271 | MLPAPKNLVVSRVTEDSARLSWTAPDAAFDSFFIYYIESYPAGEAIVLTVPGSERS |
| | YDLTGLKPGTEYWVGIDGVKGGRWSTPLSAIFTT |
| 272 | MLPAPKNLVVSRVTEDSARLSWTAPDAAFDSFFIYYIESYPAGEAIVLTVPGSCRS |
| | YDLTGLKPGTEYWVGIDGVKGGRWSTPLSAIFTT |
| 292 | MLPAPKNLVVSRVTEDSARLSWTAPDAAFDSFAIVYHEPRPDGEAIVLTVPGSCR |
| | SYDLTGLKPGTEYEVVILGVKGGVHSYPLSAIFTTAPAPAPAPAPLPAPKNLVVSR |
| | VTEDSARLSWTAPDAAFDSFAIVYHEPRPDGEAIVLTVPGSERSYDLTGLKPGTEY |
| | EVVILGVKGGVHSYPLSAIFTT |
| 293 | MLPAPKNLVVSRVTEDSARLSWTAPDAAFDSFAIVYHEPRPDGEAIVLTVPGSCR |
| | SYDLTGLKPGTEYEVVILGVKGGVHSYPLSAIFTTGGGGSGGGGSGGGGSGGGGS |
| | LPAPKNLVVSRVTEDSARLSWTAPDAAFDSFAIVYHEPRPDGEAIVLTVPGSERSY |
| | DLTGLKPGTEYEVVILGVKGGVHSYPLSAIFTT |
| 294 | MLPAPKNLVVSRVTEDSARLSWTAPDAAFDSFAIVYHEPRPDGEAIVLTVPGSCR |
| | SYDLTGLKPGTEYEVVILGVKGGVHSYPLSAIFTTEAAAKEAAAKEAAAKEAAA |
| | KLPAPKNLVVSRVTEDSARLSWTAPDAAFDSFAIVYHEPRPDGEAIVLTVPGSERS |
| | YDLTGLKPGTEYEVVILGVKGGVHSYPLSAIFTT |
| 295 | MLPAPKNLVVSRVTEDSARLSWTAPDAAFDSFAIVYHEPRPDGEAIVLTVPGSCR |
| | SYDLTGLKPGTEYEVVILGVKGGVHSYPLSAIFTTEAAAKLPAPKNLVVSRVTED |
| | SARLSWTAPDAAFDSFAIVYHEPRPDGEAIVLTVPGSERSYDLTGLKPGTEYEVVI |
| | LGVKGGVHSYPLSAIFTT |
| 296 | MLPAPKNLVVSRVTEDSARLSWTAPDAAFDSFKIEYFEYVGYGEAIVLTVPGSCR |
| | SYDLTGLKPGTEYYVAIYGVKGGWYSRPLSAIFTTAPAPAPAPAPLPAPKNLVVS |
| | RVTEDSARLSWTAPDAAFDSFKIEYFEYVGYGEAIVLTVPGSERSYDLTGLKPGTE |
| | YYVAIYGVKGGWYSRPLSAIFTT |
| 297 | MLPAPKNLVVSRVTEDSARLSWTAPDAAFDSFKIEYFEYVGYGEAIVLTVPGSCR |
| | SYDLTGLKPGTEYYVAIYGVKGGWYSRPLSAIFTTGGGGSGGGGSGGGGSGGGG |
| | SLPAPKNLVVSRVTEDSARLSWTAPDAAFDSFKIEYFEYVGYGEAIVLTVPGSERS |
| | YDLTGLKPGTEYYVAIYGVKGGWYSRPLSAIFTT |
| 298 | MLPAPKNLVVSRVTEDSARLSWTAPDAAFDSFKIEYFEYVGYGEAIVLTVPGSCR |
| | SYDLTGLKPGTEYYVAIYGVKGGWYSRPLSAIFTTEAAAKEAAAKEAAAKEAAA |
| | KLPAPKNLVVSRVTEDSARLSWTAPDAAFDSFKIEYFEYVGYGEAIVLTVPGSERS |
| | YDLTGLKPGTEYYVAIYGVKGGWYSRPLSAIFTT |
| 299 | MLPAPKNLVVSRVTEDSARLSWTAPDAAFDSFKIEYFEYVGYGEAIVLTVPGSCR |
| | SYDLTGLKPGTEYYVAIYGVKGGWYSRPLSAIFTTEAAAKLPAPKNLVVSRVTED |
| | SARLSWTAPDAAFDSFKIEYFEYVGYGEAIVLTVPGSERSYDLTGLKPGTEYYVAI |
| | YGVKGGWYSRPLSAIFTT |
| 304 | MLPAPKNLVVSRVTEDSARLSWTAPDAAFDSFAIVYHEPRPDGEAIVLTVPGSCR |
| | SYDLTGLKPGTEYEVVILGVKGGVHSYPLSAIFTT |
| 305 | MLPAPKNLVVSRVTEDSARLSWTAPDAAFDSFAIVYHEPRPDGEAIVLTVPGSER |
| | SYDLTGLKPGTEYEVVILGVKGGVHSYPLSAIFTT |
| 306 | MLPAPKNLVVSRVTEDSARLSWTAPDAAFDSFFIGYLEPQPPGEAIHLGVPGSCRS |
| | YDLTGLKPGTEYNVTIQGVKGGFPSIPLFASFTT |

### Example 7: FN3-siRNA Conjugation and Purification

ABX1005 was prepared by conjugation of cysteine modified FN3 CD71-49 (SEQ ID: 272) to ABX0214 (Table 6) via cysteine-specific chemistry using maleimide modified siRNA. For cysteine-maleimide conjugation, cysteine-containing FN3 domains in PBS at 50-200 μM were reduced with 10 mM tris(2-carboxyethyl) phosphine (TCEP) at room temperature (30 mins) to yield a free thiol. To remove the TCEP, the FN3 protein was precipitated with saturated ammonium sulfate solution and then mixed with maleimide-modified siRNA duplex dissolved in water immediately prior at a molar ratio of ˜1.5:1 FN3-protein: siRNA. After 1 hr incubation at RT or 37° C., reaction was quenched with N-ethyl maleimide (1 mM final NEM concentration in the reaction mixture).

TABLE 6
AHA1 siRNA design

| | SEQ | | SEQ | | |
|---|---|---|---|---|---|
| siRNA | ID | | ID | | |
| Pair | NO: | Sense 5-3 | NO: | Antisense 5-3 | Linker |
| ABXO214 | 286 | [mU][*mC][*fU][mC][fG][mU][fG] | 287 | [VP][*fU][*mU][fC][mA][fU][mU] | Mal- |
| | | [mG][fC][mC][fU][mU][fA][mA] | | [fA][mA][fG][mG][fC][mC][fA] | C₂H₄C(O) |
| | | [fU][mG][fA][mA][fA]-L | | [mC][fG][mA][fG][mA][*mU][*mU] | (NH)—(CH₂)₆ |

Abbreviations Key: (n/N = any nucleotide) mN = 2′-O-methyl residues, fN = 2′-F residues, * = phosphorothioate and (idt) = inverted DT, (VP) 2′-O methyl vinlyl phosphonate uridine

FN3-siRNA conjugates were purified in two steps using IMAC chromatography (HisTrap HP) to remove unreacted siRNA linker, and anion exchange chromatography-Capto-DEAE; to remove unreacted FN3 proteins. FN3-protein-siRNA conjugates were characterized by PAGE, analytical size exclusion chromatography and LC/MS. Concentration of conjugate was calculated based on absorbance of conjugate solution at 260 using a Nanodrop.

### FN3-siRNA In Vivo Activity in Mice

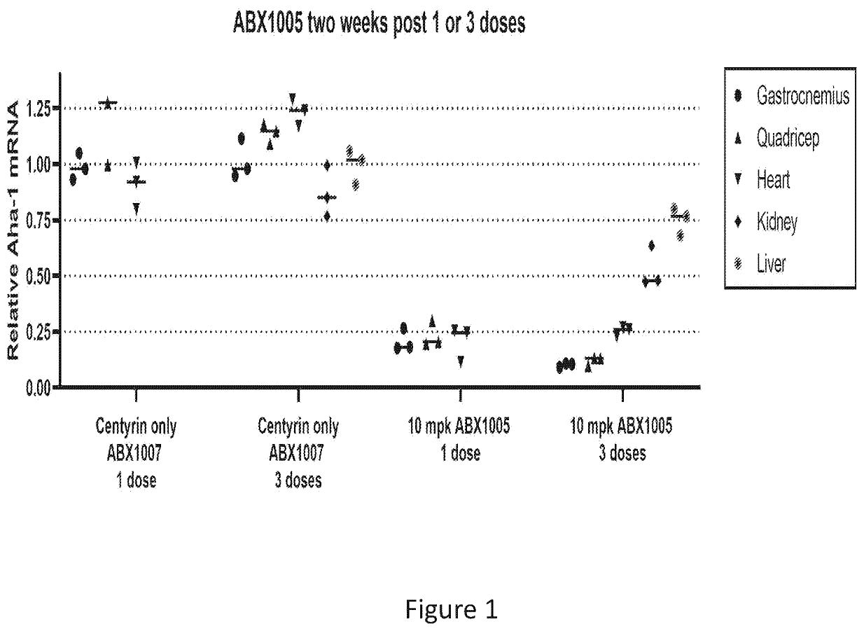

Male CD-1 mice were treated with either one or three intravenous doses of ABX1005 (CD71 FN3 domain conjugated to siRNA) at 10 mpk siRNA or ABX1007 (FN3-only control) at an equimolar dose. Tissues were collected two weeks after the final dose and were processed for AHA-1 knockdown analysis by quantitative reverse transcription polymerase chain reaction (RT-PCR). 18S ribosomal RNA was used as the RT-PCR endogenous control gene. Levels of knockdown are compared to vehicle-treated mice. siRNA-mediated knockdown of AHA-1 was observed in all muscle groups analyzed in this study (gastrocnemius, quadriceps, heart) (FIG. 1).

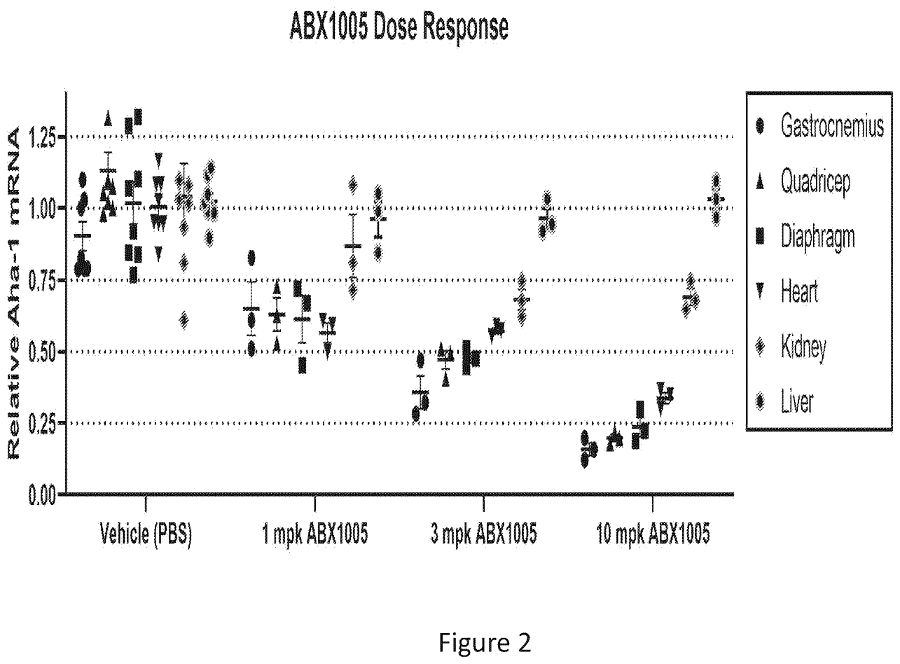

Male C57/BL6 mice were treated with a single intravenous dose of ABX1005 at 10 mpk, 3 mpk, or 1 mpk siRNA. Tissues were collected two weeks after a single dose and were processed for AHA-1 knockdown analysis by quantitative reverse transcription polymerase chain reaction (RT-PCR). 18S ribosomal RNA was used as the RT-PCR endogenous control gene. Levels of knockdown are compared to vehicle-treated mice. A dose-dependent knockdown of AHA-1 was observed in all muscle groups analyzed in this study (gastrocnemius, quadriceps, diaphragm, heart) (FIG. 2).

These examples demonstrate that siRNA molecules conjugated to the FN3 domains, such as the FN3 domains that bind to CD71 provided for herein can be used to deliver siRNA molecules, as well as other active moieties to specific tissues and regulate the expression of a specific target.

### Example 8: CD71 FN3 Domain siRNA Conjugate Binding Specificity

Integral Molecular (www.integralmolecular.com) performed their proprietary Membrane Proteome Array (MPA) assay to profile the specificity of CD71 FN3 domain and CD71 FN3 domain siRNA conjugate against the library of human membrane proteins (FIG. 3). The MPA contains over 6000 human membrane proteins, covering 94% of all single-pass, multi-pass and GPI anchored proteins including GPCRs, ion channels and transporters with each membrane protein uniquely expressed in an avian QT6 cell background. Flow cytometry was used to directly detect FN3 domain binding to membrane proteins individually expressed in unfixed cells.

FN3 domain and FN3 domain-siRNA conjugate were screened at the concentration with optimal signal/background noise ratio, 1.25 ug/ml or 0.31 ug/ml respectively, against the MPA. Membrane protein targets identified in screening were validated using ligand serial dilution on cells uniquely expressing the identified targets.

### Example 9: In Vivo Comparison of CD71 Centyrin Conjugate and CD71 Monoclonal Antibody Conjugate

Our objective in this study is to determine the duration of pharmacodynamic activity of the tool centyrin-AHA1 conjugate in comparison to a monoclonal antibody R17 conjugated with AHA1 siRNA. In C57BL6/J male mice, a single intravenous bolus of 17.9 mg tool centyrin-AHA1 siRNA conjugate containing 10 mg AHA1 siRNA or 120 mg monoclonal antibody R17 conjugated with AHA1 siRNA containing 10 mg AHA1 siRNA was administered. Gastrocnemius muscle tissue not exceeding 0.5 cm in any direction was collected at time points 2, 4 and 8 weeks post dose (N=3/time point) in RNA later to ensure good penetration of RNA later and stored at 4 C for 24 hours before storing them at −80 C. Total RNA was isolated from the gastrocnemius using Qiagen's RNeasy Fibrous Tissue kit. Using real-time, quantitative PCR, the expression levels of the target AHA1 and the endogenous control, Pgk1 were measured. Data were analyzed using the ΔΔCt method normalized to control animals dosed with vehicles alone. Gene expression levels of AHA1 and Pgk1 for each animal in the treatment groups were presented relative to an average of the 3-vehicle controls. The percentage knockdown of AHA1 mRNA in the tool AHA1-siRNA conjugate treatment groups and in the monoclonal antibody R17 conjugated with AHA1 siRNA treatment groups were measured by subtracting the percentage remaining AHA1 mRNA levels by 100.

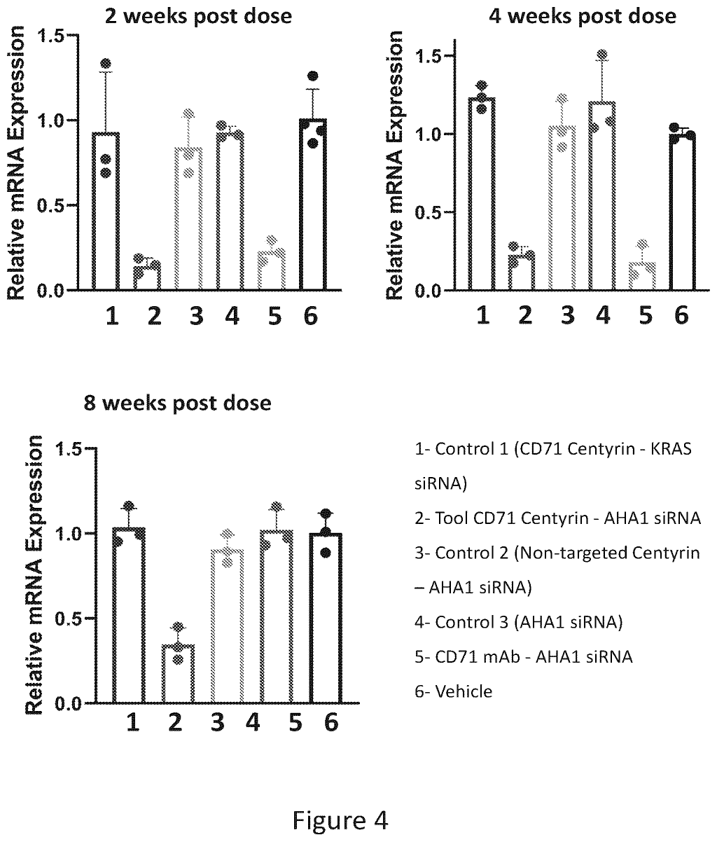

CD71 Centyrin conjugate drives sustained gene knockdown at fraction of mAb conjugate dose. C57/B6 mice received a single dose (10 mg/kg siRNA) of test conjugate. Relative RNA expression of AHA1 was measured in the gastrocnemius muscle at 2 weeks post dose, 4 weeks post dose and 8 weeks post dose. FIG. 4 and Table 7 present the data demonstrating equivalent activity for the mRNA knockdown in muscle however the CD71 Centyrin conjugate requires far less conjugate dose.

TABLE 7

| | Centyrin - siRNA | mAb - siRNA |
|---|---|---|
| | conjugate | conjugate |
| AHA knockdown | 86% | 77% |
| wk 2 | | |
| AHA knockdown | 77% | 82% |
| wk 4 | | |
| siRNA dose | 10 mg/kg | 10 mg/kg |
| (mg/kg) | | |
| Conjugate dose | 18 mg/kg | 120 mg/kg |
| (mg/kg) | | |

### Example 10: Centyrin-siRNA Conjugates are Active in Cynomolgus Monkey (Macaca fascicularis) Skeletal Muscles and Heart

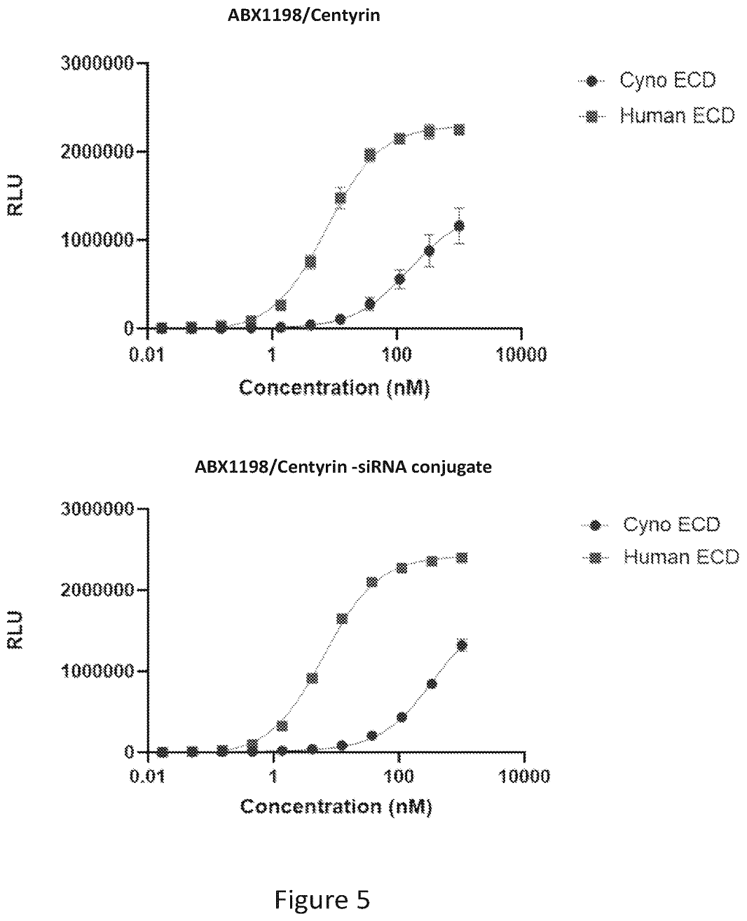

NeutrAvidin coated 96-well plates (Pierce, 15116) were washed with PBS-Tween (0.05%), and blocked for 30 minutes with blocking buffer (Starting Block T20, ThermoFisher 37539). Biotinylated antigen (human CD71-ECD [Acro Biosystems TFR-H8243] or cyno CD71-ECD [Acro Biosystems TFR-C8249]) was immobilized on blocked plates at a concentration of 20 nM, incubated for 1 hour at room temperature. Centyrin samples were diluted in blocking buffer, titrated from 1000 nM to 0.0169 nM, and incubated for 2 hours at room temperature. The plates were washed with PBS-Tween. Anti-Centyrin antibody prepared at 1:2500 in blocking buffer, added to plates, and incubated for 1 hour. The plates were washed with PBS-Tween. Anti-rabbit HRP antibody was prepared at 1:2500 in blocking buffer, added to plates, and incubated for 1 hour. The plates were washed and read-out with ELISA substrate (Roche, 11582950001) on SpectraMax Paradigm. FIG. 5 and Table 8 present the data demonstrating CD71 Centyrin as well as the CD71 Centyrin conjugate effectively bind both human and cyno CD71 and the siRNA conjugate does not interfere with CD71 Centyrin binding.

TABLE 8

| | Centyrin EC50 | Conjugate EC50 |
|---|---|---|
| Domain | (nM) | (nM) |
| Hu CD71 ECD | 7.4 | 6.3 |
| Cyno CD71 ECD | >150 | >150 |

The purpose of the present study was to determine the pharmacodynamic (PD) activity of centyrin-AHA1 siRNA conjugate in the cynomolgus monkey model. Two male cynomolgus monkeys were treated with either 17.12 mpk centyrin-AHA1 siRNA conjugate containing 10 mpk AHA1 siRNA (N=2) or vehicle (N=2) via IV bolus once a week on the right saphenous vein for three weeks. Four weeks post the last dose, skeletal muscle tissues (left and right gastrocnemius, left and right quadriceps, diaphragm, left and right biceps, soleus), smooth muscle tissue (jejunum), left and right heart and non-skeletal muscle tissues (skin, liver and kidney) were harvested and stored in RNA later to ensure good penetration of RNA later and stored at 4 C for 24 hours. Total RNA was isolated from these tissues using Qiagen's RNeasy Fibrous Tissue kit. The expression levels of the target AHA1 and endogenous controls (ARL1, ARFGAP2, HPRT1, GAPDH and Gys1) were measured by real-time, quantitative PCR. Data were analyzed using the ΔΔCt method normalized to control animals dosed with vehicles alone. The average of 2 samples (1 biopsy from each side of the tissue) or 1 sample (1 biopsy) was taken for analysis. The percentage knockdown of AHA1 mRNA in the centyrin-AHA1 siRNA conjugate treatment group and in the vehicle group were measured by subtracting the percentage remaining AHA1 mRNA levels by 100. In each tissue, the percentage of AHA1 knockdown is shown in order from the highest to the least amount of AHA1 knockdown.

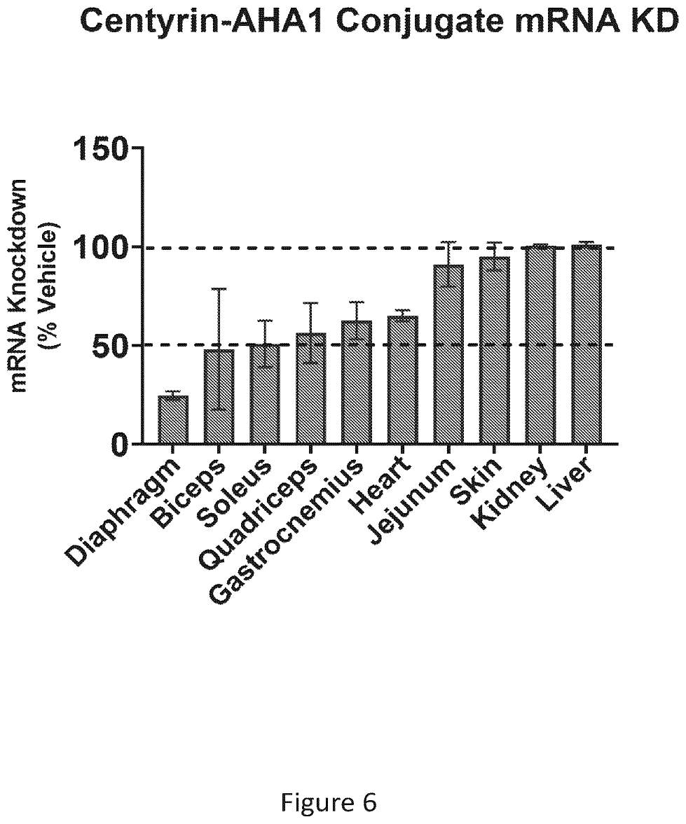

Centryin-siRNA AHA1 conjugate effectively knocked-down mRNA levels in vivo in cynomolgus monkey muscles and heart, see FIG. 6. Monkeys were dosed at 10 mg/kg siRNA 3 times per week. mRNA levels were assessed at day 28 post three doses.

### General Methods

Standard methods in molecular biology are described Sambrook, Fritsch and Maniatis (1982 & 1989 2nd Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the embodiments in addition to those described herein will become apparent to those skilled in the art from the foregoing description. Such modifications are intended to fall within the scope of the appended claims.

The foregoing written specification is considered to be sufficient to enable one skilled in the art to practice the embodiments. Various modifications of the embodiments in addition to those shown and described herein will become apparent to those skilled in the art from the foregoing description and fall within the scope of the appended claims.

1-34. (canceled)

35. A method of delivering an agent of interest to a CD71 positive cell, the method comprising contacting the cell with the agent of interest coupled to an FN3 domain that binds to CD71, wherein the FN3 domain comprises an amino acid sequence of SEQ ID NO: 209, 1-7, 10, 12-208, 210-219, 221-272, 292-299, or 304-306.

36. The method of claim 35, wherein the agent of interest is internalized into the cell through the CD71 mediated interaction.

37. The method of claim 35, wherein the FN3 domain does not compete with transferrin binding to CD71.

38. The method of claim 35, wherein the agent of interest is a chemotherapeutic agent, a drug, a growth inhibitory agent, a toxin, a radioactive isotype, an anti-tubulin agent, a polynucleotide, an oligonucleotide, an siRNA molecule, an antisense molecule, an RNA molecule, a DNA molecule, a DNA minor groove binder, a DNA replication inhibitor, an alkylating agent, an antibiotic, an antifolate, an antimetabolite, a chemotherapy sensitizer, a topoisomerase inhibitor, or a vinca alkaloid.

39. The method of claim 35, wherein the FN3 domain comprises a sequence of SEQ ID NO: 209.

40. The method of claim 35, wherein the cell is a muscle cell.

41. The method of claim 35, wherein the cell is a brain cell or cell inside of the blood brain barrier.

42-53. (canceled)

54. The method of claim 35, wherein the agent of interest can elicit one or more cytotoxic effects by modulating gene expression, RNA expression or levels, tubulin binding, DNA binding, topoisomerase inhibition, DNA cross linking, chelation, spliceosome inhibition, nicotinamide phosphoribosyltransferase (NAMPT) inhibition, or histone deacetylase (HDAC) inhibition.

55. The method of claim 35, wherein the agent of interest is an siRNA molecule comprising a sense strand and an antisense strand.

56. The method of claim 55, wherein the polypeptide is coupled to the sense strand of the siRNA molecule.

57. The method of claim 56, wherein the polypeptide is coupled to the 3′ end of the sense strand of the siRNA molecule.

58. The method of claim 56, wherein the polypeptide is coupled to the 5′ end of the sense strand of the siRNA molecule.

59. The method of claim 35, wherein the polypeptide is coupled to an antisense molecule.

60. The method of claim 35, wherein the polypeptide is coupled to an oligonucleotide.

61. The method of claim 35, wherein the polypeptide is coupled to a DNA molecule.

62. The method of claim 35, wherein the polypeptide is further coupled to a half-life extending moiety.

63. The method of claim 62, wherein the half-life extending moiety is an albumin binding molecule, a polyethylene glycol (PEG), albumin, albumin variant, an Fc region of an immunoglobulin, or a fragment thereof.


SEQUENCE LISTING (311 sequences)

SEQ ID NO: 1 - protein, 90 aa, synthetic construct
MLPAPKNLVVSRVTEDSARLSWTAPDAAFDSFPITYIEAVVLGEAIVLTVPGSERSYDLTGLKPGTEYPVGISGVKGGHNSMPLSAIFTT

SEQ ID NO: 2 - protein, 90 aa, synthetic construct
MLPAPKNLVVSEVTEDSARLSWQGVARAFDSFMINYSELFWMGEAIVLTVPGSERSYDLTGLKPGTEYVVRIKGVKGGKGSWPLHAHFTT

SEQ ID NO: 3 - protein, 90 aa, synthetic construct
MLPAPKNLVVSRVTEDSARLSWTAPDAAFDSFNIEYAETRWYGEAIVLTVPGSERSYDLTGLKPGTEYVVPIDGVKGGIASKPLSAIFTT

SEQ ID NO: 4 - protein, 90 aa, synthetic construct
MLPAPKNLVVSEVTEDSARLSWQGVARAFDSFLITYRDQIFAGEVIVLTVPGSERSYDLTGLKPGTEYPVWIQGVKGGSPSAPLSAESTT

SEQ ID NO: 5 - protein, 90 aa, synthetic construct
MLPAPKNLVVSEVTEDSARLSWQGVARAFDSFLITYREQIFAGEVIVLTVPGSERSYDLTGLKPGTEYWVYIWGVKGGKPSFPLRAGFTT

SEQ ID NO: 6 - protein, 90 aa, synthetic construct
MLPAPKNLVVSRVTEDSARLSWTAPDAAFDSFPIIYMETFSRGEAIVLTVPGSERSYDLTGLKPGTEYRVPIGGVKGGSSSCPLSAIFTT

SEQ ID NO: 7 - protein, 90 aa, synthetic construct
MLPAPKNLVVSDVTEDSARLSWQGVARAFDSFLITYREQIFAGEVIVLTVPGSERSYDLTGLKPGTEYPVWIQGVKGGSPSAPLSAEFTT

SEQ ID NO: 8-9 - skipped (2 entries)

SEQ ID NO: 10 - protein, 90 aa, synthetic construct
MLPAPKNLVVSRVTEDSARLSWTAPDAAFDSFKIAYIETATRGEAIVLTVPGSERSYDLTGLKPGTEYVVPIPGVKGGNTSSPLSAIFTT

SEQ ID NO: 11 - skipped

SEQ ID NO: 12 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAISTT

SEQ ID NO: 13 - protein, 90 aa, synthetic construct
MLPAPKNLVVSRVTEDSARLSWTAPDAAFDSFLIAYPEDGFRGEAIVLTVPGSERSYDLTGLKPGTEYPVPILGVKGGGGSGPLSAIFTT

SEQ ID NO: 14 - protein, 90 aa, synthetic construct
MLPAPKNLVVSRVTEDSARLSWTAPDAAFDSFPIYYVENVVWGEAIVLTVPGSERSYDLTGLKPGTEYWEVIIGVKGGQCSRPLSAIFTT

SEQ ID NO: 15 - protein, 90 aa, synthetic construct
MLPAPKNLVVSRVTEDSARLSWQGVARAFDSFLITYREQIFAGEVIVLTVPGSERSYDLTGLKPGTECPVWIQGVKGGSPSAPLSAEFTT

SEQ ID NO: 16 - protein, 89 aa, synthetic construct
MLPAPKNLVVSRVTEDSARLSWTAPDAAFDSFGIAYREFRPSGEAIVLTVPGSERSYDLTVETGYRNEVVICGVKGGPWSGPLSAIFTT

SEQ ID NO: 17 - protein, 90 aa, synthetic construct
MLPAPKNLVVSRVTEDSARLSWTAPDAAFDSFPILYTECVYRGEAIVLTVPGSERSYDLTGLKPGTEYHVPITGVKGGGGSWPLSAIFTT

SEQ ID NO: 18 - protein, 90 aa, synthetic construct
MLPAPKNLVVSRVTEDSARLSWTAPDAAFDSFNIMYHEIIYVGEAIVLTVPGSERSYDLTGLKPGTEYPVPIEGVKGGGTSGPLSAIFTT

SEQ ID NO: 19 - protein, 90 aa, synthetic construct
MLPAPKNLVVSRVTEDSARLSWTAPDAAFDSFAITYTEAALCGEAIVLTVPGSERSYDLTGLKPGTEYPVPINGVKGGGTSGPLSAIFTT

SEQ ID NO: 20 - protein, 90 aa, synthetic construct
MLPAPKNLVVARVTEDSARLSWTAPDAAIDSFPIDYSEYWWGGEAIVLTVPGSERSYDLTGLKPGTEYPVLITGVKGGYRSGPLSAIFTT

SEQ ID NO: 21 - protein, 90 aa, synthetic construct
MLPAPKNLVVSRVTEDSARLSWTAPDAAFDSFSIRYNEFIVAGEAIVLTVPGSERSYDLTGLKPGTEYDVPIAGVKGGGASWPLSAIVTT

SEQ ID NO: 22 - protein, 88 aa, synthetic construct
MLPAPKNLVVSRVTEDSARLSWTAPDAAFDSFWYLELQFAGEAIVLTVPGSERSYDLTGLKPGTEYNVPITGVKGGIISFPLSAIFTT

SEQ ID NO: 23 - protein, 90 aa, synthetic construct
MLPAPKNLVVSRVTEDSARLSWTAPDAAFDSFAIWYHEWYGDGEAIVLTVPGSERSYDLTGPKPGTEYRVRISGVKGGFESGPLSAIFTT

SEQ ID NO: 24 - protein, 90 aa, synthetic construct
MLPAPKNLVVSRVTEDSARLSWTAPDAAFDSFMIRYQEGTRWGEAIVLTVPGSERSYDLTGLKPGTEYIVMIAGVKGGQISLPLSAIFTT

SEQ ID NO: 25 - protein, 90 aa, synthetic construct
MLPAPKNLVVSRVTEDSARLSWTAPDAAFDSFPIWYLEKSYQGEAIVLTVPGSERSYDLTGLKPGTEYVVPIIGVKGGRDSCPLSAIFTT

SEQ ID NO: 26 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAEFTT

SEQ ID NO: 27 - protein, 89 aa, synthetic construct
MLPAPKNLVVSRVTEDSARLSWTAPDAAFDSFRISYAETVRQGEAIVLTVPGSERSYDLTVETGYRNWVMILGVKGGPGSLPLSAIFTT

SEQ ID NO: 28 - protein, 90 aa, synthetic construct
MLPAPKNLVVSEVTEDSARLSWQGVVRAFDSFLITYREQIFAGEVIVLTVPGSERSYDLTGLKPGTEYPVWIQGVKGGSPSAPLSAEFTT

SEQ ID NO: 29 - protein, 90 aa, synthetic construct
MLPAPKNLVVSRVTEDSARLSWTAPDAAFDSFWIEYWEAVGFGEAIVLTVPGSERSYDLTGLKPGTEYFVGIYGVKGGYLSAPLSAIFTT

SEQ ID NO: 30 - protein, 90 aa, synthetic construct
MLPAPKNLVVSRVTEDSARLSWTAPDAAFDSFIIHYVEQQLIGEAIVLTVPGSERSYDLTGLKPGTEYPVPITGVKGGACSWPLSAIFTT

SEQ ID NO: 31 - protein, 90 aa, synthetic construct
MLPAPKNLVVSRVTEDSARLSWTAPDAAFDSFTIEYSEHPIDGEAIPLFVPGSERSYDLTGLKPGTEYYVRIHGVKGGWFSHPLWAFFTT

SEQ ID NO: 32 - protein, 90 aa, synthetic construct
MLPAPKNLVVSRVTEDSARLSWQGVARAFDSFLITYREQIFAGEVIVLTVPGSERSYDLTGLKPGTEYGVTIAGVKGGWRSKPLNAESTT

SEQ ID NO: 33 - protein, 90 aa, synthetic construct
MLPAPKNLVVSRVTEDSARLSWTAPDAAFDSFGIAYVESYWYGEAIVLTVPGSERSYDLTGLKPGTEYNVPIYGVKGGDGSGPLSAIFTT

SEQ ID NO: 34 - protein, 90 aa, synthetic construct
MLPAPKNLVVSRVTEDSARLSWTAPDAAFDSFYITYVELNLAGEAIVLTVPGSERSYDLTGLKPGTEYPVPILGVKGGSLSQPLSAIFTT

SEQ ID NO: 35 - protein, 90 aa, synthetic construct
MLPAPKNLVVSRVTEDSARLSWTAPDAAFDSFPISYIESIADGEAIVLTVPGSERSYDLTGLKPGTEYWVAIVGVKGGPFSWSLSAIVTT

SEQ ID NO: 36 - protein, 90 aa, synthetic construct
MLPAPKNLVVSEVTEDSARLSWQGVARAFDSFLITYREQIFAGEVIVPTVPGSERSYDLTGLKPGTEYPVPIAGVKGGGPSAPLSAIFTT

SEQ ID NO: 37 - protein, 90 aa, synthetic construct
MLPAPKNLVVSRVTEDSARLSWTTPDAAFDSFPIYYWEVTITGEAIYLSVPGSERSYDLTGLKPGTEYPVDIPGVKGGAASPPLSAIFTT

SEQ ID NO: 38 - protein, 90 aa, synthetic construct
MLPAPKNLVVSRVTEDSARLSWTAPDAAFDSFPILYLEHTVRSEAIVLTVPGSERSYDLTDLKPGTEYCVPIDGVKGGLRSRPLSAIFTT

SEQ ID NO: 39 - protein, 90 aa, synthetic construct
MLPAPKNLVVSRVTEDSARLSWTAPDAAFDSFPIPYTEPPDPGEAIVLTVPGSERSYDLTGLKPGTEYLVTILGVKGGSMSVPLSAIFTT

SEQ ID NO: 40 - protein, 90 aa, synthetic construct
MLPAPKNLVVSRVTEDSARLSWTAPDAAFDSFTIDYWENRCPGEAIVLTVPGSERSYDLTGLKPGTEYCVWISGVKGGYSSWPLSAIFTT

SEQ ID NO: 41 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGHLSDPLSAIVTT

SEQ ID NO: 42 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSEPLSAIFTT

SEQ ID NO: 43 - protein, 90 aa, synthetic construct
MLPAPKNLVVSRVTEDSARLSWTAPDAAFDSFMIVYYEYTRFGEAIVLTVPGSERSYDLTGLKPGTEYTVPIDGVKGGGRSSPLSAIFTT

SEQ ID NO: 44 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAIVTT

SEQ ID NO: 45 - protein, 90 aa, synthetic construct; identical to SEQ ID NO: 26

SEQ ID NO: 46 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGKLSLPLSAIFTT

SEQ ID NO: 47 - protein, 90 aa, synthetic construct
MLPAPKNLVVSRVTEDSARLSWTAPDAAFDSFGIVYLEMMVTGEAIVLTVPGSERSYDLTGLKPGTEYDVPILGVKGGTRSVPLSAIFTT

SEQ ID NO: 48 - protein, 94 aa, synthetic construct
MLPAPKNLVVSRVTEDSARLSWTAPDAAFDSFLIYYEEGYLEYYYSGEAIVLTVPGSERSYDLTGLKPGTEYYVGIVGVKGGGLSGPLSAISTT

SEQ ID NO: 49 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGDWSLPLSAIFTT

SEQ ID NO: 50 - protein, 91 aa, synthetic construct
MSLPAPKNLVVSRVTEDSARLSWTAPDAAFDSFTIHYREFQLSGEAIVLTVPGSERSYDLTGLKPGTEYDVPIEGVKGGPGSRPLSAIFTT

SEQ ID NO: 51 - protein, 90 aa, synthetic construct
MLPAPKNLVVSEVTEDSARLSWQGVARAFDSFLITYREQIFAGEVIVLTVPGSECSYDLTGLKPGTEYPVWIQGVKGGSPSAPLSAEFTT

SEQ ID NO: 52 - protein, 90 aa, synthetic construct
MLPAPKNLVVSRVTEDSARLSWTAPDAAFDSFEIDYDELAIYGEAIVLTVPGSERSYDLTGLKPGTEYGVRIPGVKGGMPSLPLSAIVTT

SEQ ID NO: 53 - protein, 90 aa, synthetic construct
MLPAPENLVVSEVTEDSARLSWQGVARAFDSFLITYREQIFAGEVIVLTVPGSERSYDLTGLKPGTEYPVWIQGVKGGSPSAPLSAESTT

SEQ ID NO: 54 - protein, 90 aa, synthetic construct
MLPAPKNLVVSRVTEDSARLSWTAPDAAFDSFAIAYGEHIVIGEAIVLTVPGSERSYDLTGLKPGTEYMVPIAGVKGGPISLPLSAIFTT

SEQ ID NO: 55 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSAIFTT

SEQ ID NO: 56 - protein, 90 aa, synthetic construct
MLPAPKNLVVSRVTEDSARLSWTAPDAAFDSFSIGYVELVLLGEAIVLTVPGSERSYDLTGLKPGTEYDVLIPGVKGGSLSRPLSAIFTT

SEQ ID NO: 57 - protein, 90 aa, synthetic construct
MLPAPKNLVVSRVTEDSARLSWTAPDAAFDSFAIPYAELSRNGEAIVLTVPGSERSYDLTGLKPGTEYTVLIHGVKGGCLSDPLSAIFTT

SEQ ID NO: 58 - protein, 90 aa, synthetic construct
MLPAPKNLVVSRVTEDSARLSWTAPDAAFDSFHIEYLELSRHGEAIVLTVPGSERSYDLTGLKPGTEYWVMIFGVKGGGPSKPLSAIFTT

SEQ ID NO: 59 - protein, 88 aa, synthetic construct
MLPAPKNLVVSRVTEDSARLSWTAPDAAFDSFVYNEVHWIGEAIVLTVPGSERSYDLTGLKPGTEYFVGIYGVKGGHWSKPLSAIFTT

SEQ ID NO: 60 - protein, 90 aa, synthetic construct; identical to SEQ ID NO: 52

SEQ ID NO: 61 - protein, 90 aa, synthetic construct
MLPAPKNLVVSRVTEDSARLSWTAPDAAFDSFQIVYSELWIKGEAIVLTVPGSERSYDLTGLKPGTEYQVPIPGVKGGRNSFPLSAIFTT

SEQ ID NO: 62 - protein, 90 aa, synthetic construct
MLPAPKNLVVSRVTEDSARLSWTAPDAAFDSFKIRYTETRSIGEAIVLTVPGSERSYDLTGLKPGTEYCVPIGGVKGGDSSWPLSAISTT

SEQ ID NO: 63 - protein, 90 aa, synthetic construct
MLPAPKNLVVSRVTEDSARLSWTAPDAAFDSFCISYYERMGRGEAIVLTVPGSERSYDLTGLKPGTEYMVYIFGVKGGLNSLPLSAIFTT

SEQ ID NO: 64 - protein, 90 aa, synthetic construct
MLPAPKNLVVSRVTEDSARLSWTAPDAAFDSFYIVYAEPIPNGEAIVLTVPGSERSYDLTGLKPGTEYSVLIHGVKGGRNSDPLSAIFTT

SEQ ID NO: 65 - protein, 90 aa, synthetic construct
MLPAPKNLVVSRVTKDSARLSWTAPDAAFDSFPIAYAEPRPDGEAIVLTVPGSERSYDLTGLKPGTEYSVLIHGVKGGLLSSPLSAIFTT

SEQ ID NO: 66 - protein, 90 aa, synthetic construct
MLPAPKNLVVSRVTEDSARLSWTAPDAAFDSFTIDYDEPRSPGEAIVLTVPGSERSYDLTGLKPGTEYRVFIWGIKGGDTSFPLSAIFTT

SEQ ID NO: 67 - protein, 88 aa, synthetic construct
MLPAPKNLVVSRVTEDSARLSWTAPDAAFDSFTILYAEQAQFGEAIVLTVPGSERSYDLTGLKPGTEYPITGVKGGTRSGPLSAISTT

SEQ ID NO: 68 - protein, 90 aa, synthetic construct
MLPAPKNLVVSRVTEDSARLSWTAPDAAFDSFIIPYAEVRPDGEAIVLTVPGSERSYDLTGLKPGTEYSVLIHGVKGGHLSDPLSAISTT

SEQ ID NO: 69 - protein, 90 aa, synthetic construct
MLPAPKNLVVSRVTEDSARLSWTAPDAAFDSFHIAYEETATSGEAIYLRVPGSERSYDLTGLKPGTEYGVEIEGVKGGARSRPLYADFTT

SEQ ID NO: 70 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGDLSNPLSAIFTT

SEQ ID NO: 71 - protein, 90 aa, synthetic construct
MLPAPKNLVVSRVTEDSARLSWTAPDAAFDSFPISYLELSLYGEAIVLTVPGSERSYDLTGLKPGTEYPVGIAGVKGGVVSRPLSAIFTT

SEQ ID NO: 72 - protein, 90 aa, synthetic construct
MLPAPKNLVVSRVTEDSARLSWTAPDAAFDSFTIGYREWYWYGEAIVLTVPGSERSYDLTGLKPGTEYNVPISGVKGGLDSFPLSAIFTT

SEQ ID NO: 73 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAESTT

SEQ ID NO: 74 - protein, 90 aa, synthetic construct
MLPAPKNLVVSRVTEDSARLSWTAPDAAFDSFSITYLEWWNLGEAIVLTVPGSERSYDLTGLKPGTEYMVTIPGVKGGMSSYPLSAIFTT

SEQ ID NO: 75 - protein, 90 aa, synthetic construct
MLPAPKNLVVSRVTEDSARLSWTAPDAAFDSFTISYGEEALIGEAIYLRVPGSERSYDLTGLKPGTEYYVHIEGVKGGSWSQPLAAAFTT

SEQ ID NO: 76 - protein, 90 aa, synthetic construct
MLPAPKNLVVSRVTEDSARLSWTAPDAAFDSFTIEYYENIGIGEAIVLTVPGSERSYDLTGLKPGTEYSVPIVGVKGGPYSHPLSAIFTT

SEQ ID NO: 77 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAIFTT

SEQ ID NO: 78 - protein, 90 aa, synthetic construct
MLPAPKNLVVSRVTEDSARLSWTAPDAAFDSFGIGYYEHKRFGEAIQLSVPGSERSYDLTGLKPGTEYEVDIEGVKGGVLSWPLFAEFTT

SEQ ID NO: 79 - protein, 90 aa, synthetic construct
MLPAPKNLVVSRVTEDSARLSWTAPDAAFDSFVIEYTERFWSGEAIVLTVPGSERSYDLTGLKPGTEYSVPIDGVKGGQCSTPLSAIFTT

SEQ ID NO: 80 - protein, 90 aa, synthetic construct
MLPAPKNLVVSRVTEDSARLSWTAPDAAFDSFWIDYEEEGVIGEAIYLHVPGSERSYDLTGLKPGTEYVVKIHGVKGGHPSHPLVAVFTT

SEQ ID NO: 81 - protein, 90 aa, synthetic construct
MLPAPKNLVVSRVTEDSARLSWQGVARAFDSFLITYVELRHLGEAIVLTVPGSERSYDLTGLKPGTEYPVWIQGVKGGSPSAPLSAEFTT

SEQ ID NO: 82 - protein, 90 aa, synthetic construct
MLPAPKNLVVSRVTEDSARLSWTAPDAAFDSFLIPYAETSPSGEAIVLTVPGSERSYDLTGLKPGTEYSVLIHGVKGGDYSSPLSAIFTT

SEQ ID NO: 83 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAISTT

SEQ ID NO: 84 - protein, 90 aa, synthetic construct
MLPAPKNLVVSRVTEDSARLSWQGVARAFDSFSILYLELTPKGEAIVLTVPGSERSYDLTGLKPGTEYPVWIQGVKGGSPSAPLSAEFTT

SEQ ID NO: 85 - protein, 90 aa, synthetic construct
MLPAPKNLVVSRVTEDSARLSWTAPDAAFDSFIIEYFEPIPIGEAIVLTVPGSERSYDLTGLKPGTEYAVNIYGVKGGYLSHPLSAIFTT

SEQ ID NO: 86 - protein, 90 aa, synthetic construct; identical to SEQ ID NO: 51

SEQ ID NO: 87 - protein, 90 aa, synthetic construct
MLPAPKNLVVSRVTEDSARLSWTAPDAAFDSFAIEYTEFLYSGEAIVLTVPGSERSYDLTGLKPGTEYGVPINGVKGGFVSPPLSAIVTT

SEQ ID NO: 88 - protein, 90 aa, synthetic construct
MLPAPKNLVVSRVTEDSARLSWTAPDAAFDSFPIKYREVLRCGEAIVLTVPGSERSYDLTGLKPGTEYTVPITGVKGGFGSSPLSAIFTT

SEQ ID NO: 89 - protein, 90 aa, synthetic construct
MLPAPENLVVSRVTEDSARLSWTAPDAAFDSFWIEYYEGVIQGEAIVLTVPGSERSYDLTGLKPGTEYFVAIWGVKGGKWSVPLSAIFTT

SEQ ID NO: 90 - protein, 86 aa, synthetic construct
MLPAPKNLVVSRVTEDSARLSWQGVARAFDSFLITYREQIFAGEVIVLTVPGSERSYDLTGLKPGTEYPVWIQGVKGGSPSAEFTT

SEQ ID NO: 91 - protein, 90 aa, synthetic construct
MLPAPKNLVVSRVTEDSARLSWTAPDAAFDSFQIHYWETQGFGEAIVLTVPGSERSYDLTGLKPGTEYPVLIPGVKGGPSSLPLSAIFTT

SEQ ID NO: 92 - protein, 90 aa, synthetic construct; identical to SEQ ID NO: 55

SEQ ID NO: 93 - protein, 90 aa, synthetic construct
MLPAPKNLVVSRVTEDSARLSWTAPDAAFDSFAIEYYEPVPAGEAIYLDVPGSERSYDLTGLKPGTEYDVTIYGVKGGYYSHPLFASFTT

SEQ ID NO: 94 - protein, 90 aa, synthetic construct
MLPAPKNLVVSEVTEDSARLSWQGVARAFDSFLITYREQIFAGEVIVLTVPGSERSYDLTGLKPGTEYPVWIQGVKGGSPSAPLSAIFTT

SEQ ID NO: 95 - protein, 90 aa, synthetic construct; identical to SEQ ID NO: 26

SEQ ID NO: 96 - protein, 91 aa, synthetic construct
MSLPAPKNLVVSRVTEDSARLSWTAPDAAFDSFPIAYLEVFYEGEAIVLTVPGSERSYDLTGLKPGTEYQVPIEGVKGGAMSLPLSAIFTT

SEQ ID NO: 97 - protein, 90 aa, synthetic construct
MLPAPKNLVVSRVTEDSARLSWTAPDAAFDSFHIWYEEETTIGEAIYLHVPGSERSYDLTGLKPGTEYEVHITGVKGGPYSRPLFANFTT

SEQ ID NO: 98 - protein, 90 aa, synthetic construct
MLPAPKNLVVSRVTEDSARLSWTAPDAAFDSFGIAYDEWPEFGEAIVLTVPGSERSYDLTGLKPDTEYIVEIYGVKGGWFSWPLSAIFTT

SEQ ID NO: 99 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGHLSDPLSVIFTT

SEQ ID NO: 100 - protein, 90 aa, synthetic construct
MLPAPKNLVVSRVTEDSARLSWTAPDAAFDSFYIWYEEVMYLGEAIVLTVPGSERSYDLTGLKPGTEYNVPIPGVKGGHSSPPLSAIFTT

SEQ ID NO: 101 - protein, 90 aa, synthetic construct
MLPAPKNLVVSRVTEDSARLSWTAPDAAFDSFHILYEELFLVGEAIVLTVPGSERSYDLTGLKPGTEYKVPISGVKGGPVSRPLSAIFTT

SEQ ID NO: 102 - protein, 90 aa, synthetic construct
MLPAPKNLVVSRVTEDSARLSWQGVARAFDSFLITYREQIFAGEVIVLTVPGSERSYDLTGLKPGTEYPVWIQGVKGGSPSAPLSAEFTT

SEQ ID NO: 103 - protein, 90 aa, synthetic construct
MLPAPKNLVVSRVTEDSARLSWTAPDAAFDSFHIVYHEPRPSGEAIWLHVPGSERSYDLTGLKPGTEYEVGIVSVKGGDLSVPLVAFFTT

SEQ ID NO: 104 - protein, 90 aa, synthetic construct
MLPAPKNLVVSRVTEDSARLSWTAPDAAFDSFYIAYAEPRPDGEAISLLVPGSERSYDLTGLKPGTEYSVLIHGVKGGLLSSPLYAVFTT

SEQ ID NO: 105 - protein, 90 aa, synthetic construct
MLPAPKNLVVSRVTEDSARLSWTAPDAAFDSFYIAYAEPRPDGEAIFLVVPGSERSYDLTGLKPGTEYSVLIHGVKGGLLSSPLHANFTT

SEQ ID NO: 106 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGLLSSPLYASFTT

SEQ ID NO: 107 - protein, 91 aa, synthetic construct
MSLPAPKNLVVSRVTEDSARLSWTAPDAAFDSFYIAYAEPRPDGEAISLYVPGSERSYDLTGLKPGTEYSVLIHGVKGGLLSSPLSAISTT

SEQ ID NO: 108 - protein, 90 aa, synthetic construct
MLPAPKNLVVSRVTEDSARLSWTAPDAAFDSFFIGYLEPQPPGEAIQLRVPGSERSYDLTGLKPGTEYNVTIQGVKGGFPSMPLSAIFTT

SEQ ID NO: 109 - protein, 90 aa, synthetic construct
MLPAPKNLVVSRVTEDSARLSWTAPDAAFDSFHISYEEDYTFGEAIYLRVPGSERSYDLTGLKPGTEYRVVIGGVKGGWFSEPLLAAFTT

SEQ ID NO: 110 - protein, 90 aa, synthetic construct
MLPAPKNLVVSRVTEDSARLSWTAPDAAFDSFFIGYLEPQPPGEAIYLTVPGSERSYDLTGLKPGTEYNVTIQGVKGGFPSYPLDASFTT

SEQ ID NO: 111 - protein, 93 aa, synthetic construct
MLPAPKNLVVSRVTEDSARLSWTAPDAAFDSFFIGYLEPQPPGEAIDLGVPGSERSYDLTGLKPGTEYNVTIQGVKGGFPSMPLDPLEAYFTT

SEQ ID NO: 112 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLSAIFTT

SEQ ID NO: 113 - protein, 90 aa, synthetic construct
MLPAPKNLVVSRVTEDSARLSWTAPDAAFDSFYIAYAEPRPDGEAINLQVPGSERSYDLTGLKPGTEYSVLIHGVKGGLLSSPLVAFFTT

SEQ ID NO: 114 - protein, 90 aa, synthetic construct
MLPAPKNLVVSRVTEDSARLSWTTPDAAFDSFFIGYLEPQPPGEAISLQVPGSERSYDLTGLKPGTEYNVTIQGVKGGFPSSPLFAVFTT

SEQ ID NO: 115 - protein, 87 aa, synthetic construct
MLPAPKNLVVSRVTEDSARLSWTAPDAAFDSFYIAYAEPRPDGEAIELHVPGSERSYDLTGLKPGTEYSVLIHGVKGGLLSSPLFTT

SEQ ID NO: 116 - protein, 90 aa, synthetic construct
MLPAPKNLVVSRVTEDSARLSWTAPDAAFDSFYIAYAEPRPDGEAIQLVVPGSERSYDLTGLKPGTEYSVLIHGVKGGLLSSPLSAIFTT

SEQ ID NO: 117 - protein, 90 aa, synthetic construct
MLPAPKNLVVSRVTEDSARLSWTAPDAAFDSFYIAYAEPRPDGEAITLDVPGSERSYDLTGLKPGTEYSVLIHGVKGGLLSSPLSAIFTT

SEQ ID NO: 118 - protein, 90 aa, synthetic construct
MLPAPKNLVVSRVTEDSARLSWTAPDAAFDSFYIAYAEPRPDGEAIWLVVPGSERSYDLTGLKPGTEYSVLIHGVKGGLLSSPLVASFTT

SEQ ID NO: 119 - protein, 90 aa, synthetic construct
MLPAPKNLVVSRVTEDSARLSWTAPDAAFDSFYIAYAEPRPDGEAINLDVPGSERSYDLTGLKPGTEYSVLIHGVKGGLLSSPLVAEFTT

SEQ ID NO: 120 - protein, 90 aa, synthetic construct
MLPAPKNLVVSRVTEDSARLSWTAPDAAFDSFYIAYAEPRPDGEAIHLSVPGSERSYDLTGLKPGTEYSVLIHGVKGGLLSSPLVAIFTT

SEQ ID NO: 121 - protein, 90 aa, synthetic construct
MLPAPKNLVVSRVTEDSARLSWTAPDAAFDSFFIGYLEPQPPGEAIALWVPGSERSYDLTGLKPGTEYNVTIQGVKGGFPSMPLSAIFTT

SEQ ID NO: 122 - protein, 90 aa, synthetic construct
MLPAPKNLVVSRVTEDSARLSWTAPDAAFDSFYIAYAEPRPDGEAIILVVPGSERSYDLTGLKPGTEYSVLIHGVKGGLLSSPLSAHFTT

SEQ ID NO: 123 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSHPLGAVFTT

SEQ ID NO: 124 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLLASFTT

SEQ ID NO: 125 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLVAFFTT

SEQ ID NO: 126 - protein, 90 aa, synthetic construct
MLPAPKNLVVSRVTEDSARLSWTAPDAAFDSFFIGYLEPQPPGEAIHLHVPGSERSYDLTGLKPGTEYNVTIQGVKGGFPSIPLHANFTT

SEQ ID NO: 127 - protein, 90 aa, synthetic construct
MLPAPKNLVVSRVTEDSARLSWTAPDAAFDSFYIAYAEPRPDGEAIFLGVPGSERSYDLTGLKPGTEYSVLIHGVKGGLLSSPLSAIFTT

SEQ ID NO: 128 - protein, 90 aa, synthetic construct
MLPAPKNLVVSRVTEDSARLSWTAPDAAFDSFYIAYAEPRPDGEAIVLRVPGSERSYDLTGLKPGTEYSVLIHGVKGGLLSSPLIASFTT

SEQ ID NO: 129 - protein, 90 aa, synthetic construct
MLPAPKNLVVSRVTEDSARLSWTAPDAAFDSFYIAYAEPRPDGEAINLWVPGSERSYDLTGLKPGTEYSVLIHGVKGGLLSSPLDASFTT

SEQ ID NO: 130 - protein, 90 aa, synthetic construct
MLPAPKNLVVSRVTEDSARLSWTAPDAAFDSFVIEYFEWTLNGEAIVLTVPGSERSYDLTGLKPGTEYSVQIYGVKGGCLSRPLSAIFTT

SEQ ID NO: 131 - protein, 90 aa, synthetic construct
MLPAPKNLVVSRVTEDSARLSWTAPDAAFDSFYIAYAEPRPDGEAIHLWVPGSERSYDLTGLKPGTEYSVLIHGVKGGLLSSPLIAHFTT

SEQ ID NO: 132 - protein, 90 aa, synthetic construct
MLPAPKNLVVSRVTEDSARLSWTAPDAAFDSFFIPYAEPSPTGEAIVLTVPGSERSYDLTGLKPGTEYSVLIHGVKGGLLSSPLSAHFTT

SEQ ID NO: 133 - protein, 90 aa, synthetic construct
MLPAPKNLVVSRVTEDSARLSWTAPDAAFDSFYIAYAEPRPDGEAIYLYVPGSERSYDLTGLKPGTEYSVLIHGVKGGLLSSPLDAFFTT

SEQ ID NO: 134 - protein, 90 aa, synthetic construct
MLPAPKNLVVSRVTEDSARLSWTAPDAAFDSFYIAYAEPRPDGEAIGLQVPGSERSYDLTGLKPGTEYSVLIHGVKGGLLSSPLSAIFTT

SEQ ID NO: 135 - protein, 90 aa, synthetic construct
MLPAPKNLVVSRVTEDSARLSWTAPDAAFDSFYIAYAEPRPDGEAIQLAVPGSERSYDLTGLKPGTEYSVLIHGVKGGLLSSPLHAFFTT

SEQ ID NO: 136 - protein, 90 aa, synthetic construct
MLPAPKNLVVSRVTEDSARLSWTAPDAAFDSFFIGYLEPQPPGEAIWLHVPGSERSYDLTGLKPGTEYNVTIQGVKGGFPSIPLIAIFTT

SEQ ID NO: 137 - protein, 90 aa, synthetic construct
MLPAPKNLVVSRVTEDSARLSWTAPDAAFDSFYIAYAEPRPDGEAIVLDVPGSERSYDLTGLKPGTEYSVLIHGVKGGLLSSPLVAEFTT

SEQ ID NO: 138 - protein, 90 aa, synthetic construct
MLPTPKNLVVSRVTEDSARLSWTAPDAAFDSFYIAYAEPRPDGEAIQLRVPGSERSYDLTGLKPGTEYSVLIHGVKGGLLSSPLHASFTT

SEQ ID NO: 139 - protein, 90 aa, synthetic construct
MLPAPKNLVVSRVTEDSARLSWTAPDAAFDSFFIGYLEPQPPGEAIQLGVPGSERSYDLTGLKPGTEYNVTIQGVKGGFPSHPLNANFTT

SEQ ID NO: 140 - protein, 90 aa, synthetic construct
MLPAPKNLVVSRVTEDSARLSWTAPDAAFDSFFIGYLEPQPPGEAIQLEVPGSERSYDLTGLKPGTEYNVTIQGVKGGFPSMPLSAIFTT

SEQ ID NO: 141 - protein, 90 aa, synthetic construct
MLPAPKNLVVSRVTEDSARLSWTAPDAAFDSFYIAYAEPRPDGEAIFLGVPGSERSYDLTGLKPGTEYSVLIHGVKGGLLSSPLIAFFTT

SEQ ID NO: 142 - protein, 90 aa, synthetic construct
MLPAPKNLVVSRVTEDSARLSWTAPDAAFDSFFIGYLEPQPPGEAIGLQVPGSERSYDLTGLKPGTEYNVTIQGVKGGFPSHPLKAQFTT

SEQ ID NO: 143 - protein, 90 aa, synthetic construct
MLPAPKNLVVSRVTEDSARLSWTAPDAAFDSFYIAYAEPRPDGEAILLFVPGSERSYDLTGLKPGTEYSVLIHGVKGGLLSSPLVAHFTT

SEQ ID NO: 144 - protein, 90 aa, synthetic construct
MLPAPKNLVVSRVTEDSARLSWTAPDAAFDSFYIAYAEPRPDGEAIGLYVPGSERSYDLTGLKPGTEYSVLIHGVKGGLLSSPLGAFFTT

SEQ ID NO: 145 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLTAIFTT

SEQ ID NO: 146 - protein, 90 aa, synthetic construct
MLPAPKNLVVSRVTEDSARLSWTAPDAAFDSFYIAYAEPRPDGEAITLHVPGSERSYDLTGLKPGTEYSVLIHGVKGGLLSSPLSAIFTT

SEQ ID NO: 147 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLDAHFTT

SEQ ID NO: 148 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLRAVFTT

SEQ ID NO: 149 - protein, 90 aa, synthetic construct
MLPAPKNLVVSRVTEDSARLSWTAPDAAFDSFYIAYAEPRPDGEAIQLWVPGSERSYDLTGLKPGTEYSVLIHGVKGGLLSSPLSAIFTT

SEQ ID NO: 150 - protein, 90 aa, synthetic construct
MLPAPKNLVVSRVTEDSARLSRTAPDAAFDSFYIAYAEPRPDGEAIVLIVPGSERSYDLTGLKPGTEYSVLIHGVKGGLLSSPLSAIFTT

SEQ ID NO: 151 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSRPLQAHFTT

SEQ ID NO: 152 - protein, 90 aa, synthetic construct
MLPAPKNLVVSRVTEDSARLSWTAPDAAFDSFYIAYAEPRPDGEAITLDVPGSERSYDLTGLKPGTEYSVLIHGVKGGLLSSPLYAFFTT

SEQ ID NO: 153 - protein, 90 aa, synthetic construct
MLPAPKNLVVSRVTEDSARLSWTAPDAAFDSFYIAYAEPRPDGEAIALHVPGSERSYDLTGLKPGTEYSVLIHGVKGGLLSSPLSAIFTT

SEQ ID NO: 154 - protein, 90 aa, synthetic construct
MLPAPKNLVVSRVTEDSARLSWTAPDAAFDSFYIAYAEPRPDGEAIGLWVPGSERSYDLTGLKPGTEYSVLIHGVKGGLLSSPLIAHFTT

SEQ ID NO: 155 - protein, 90 aa, synthetic construct
MLPAPKNLVVSRVTEDSARLSWTAPDAAFDSFYIAYAEPRPDGEAIWLVVPGSERSYDLTGLKPGTEYSVLIHGVKGGLLSSPLHARFTT

SEQ ID NO: 156 - protein, 90 aa, synthetic construct
MLPAPKNLVVSRVTEDSARLSWTAPDAAFDSFYIAYAEPRPDGEAIFLQVPGSERSYDLTGLKPGTEYSVLIHGVKGGLLSSPLAAVFTT

SEQ ID NO: 157 - protein, 90 aa, synthetic construct
MLPAPKNLVVSRVTEDSARLSWTAPDAAFDSFYIAYAEPRPDGEAILLHVPGSERSYDLTGLKPGTEYSVLIHGVKGGLLSSPLSAISTT

SEQ ID NO: 158 - protein, 90 aa, synthetic construct
MLPAPKNLVVSRVTEDSARLSWTAPDAAFDSFYIAYAEPRPDGEAIILQVPGSERSYDLTGLKPGTEYSVLIHGVKGGLLSSPLSAVFTT

SEQ ID NO: 159 - protein, 90 aa, synthetic construct
MLPAPKNLVVSRVTEDSARLSWTAPDAAFDSFYIAYAEPRPDGEAIYLKVPGSERSYDLTGLKPGTEYSVLIHGVKGGLLSSPLVAHFTT

SEQ ID NO: 160 - protein, 90 aa, synthetic construct
MLPAPKNLVVSRVTEDSARLSWTAPDAAFDSFYIAYAEPRPDGEAIVLTVPGSERSYDLTGLKPGTEYSVLIHGVKGGLLSSPLLAYFTT

SEQ ID NO: 161 - protein, 90 aa, synthetic construct
MLPAPKNLVVSRVTEDSARLSWTAPDAAFDSFYIAYAEPRPDGEAIILHVPGSERSYDLTGLKPGTEYSVLIHGVKGGLLSSPLEAKFTT

SEQ ID NO: 162 - protein, 89 aa, synthetic construct
MLPAPKNLVVSRVTEDSARLSWTAPDAAFDSFYIAYAEPRPDGEAIKLEVPGSERSYDLTGLKPGTEYSVLIHGVKGGLLSSPLAIFTT

SEQ ID NO: 163 - protein, 90 aa, synthetic construct
MLPAPKNLVVSRVTEDSARLSWTAPDAAFDSFFIGYLEPQPPGEAIYLEVPGSERSYDLTGLKPGTEYNVTIQGVKGGFPSFPLKAAFTT

SEQ ID NO: 164 - protein, 90 aa, synthetic construct
MLPAPKNLVVSRVTEDSARLSWTAPDAAFDSFYIAYAEPRPDGEAIILRVPGSERSYDLTGLKPGTEYSVLIHGVKGGLLSSPLVAIFTT

SEQ ID NO: 165 - protein, 90 aa, synthetic construct
MLPAPKNLVVSRVTEDSARLSWTAPDAAFDSFYIAYAEPRPDGEAIVLQVPGSERSYDLTGLKPGTEYSVLIHGVKGGLLSSPLAAWFTT

SEQ ID NO: 166 - protein, 90 aa, synthetic construct
MLPAPKNLVVSRVTEDSARLSWTAPDAAFDSFYIAYAEPRPDGEAIFLQVPGSERSYDLTGLKPGTEYSVLIHGVKGGLLSSPLNAFFTT

SEQ ID NO: 167 - protein, 90 aa, synthetic construct
MLPAPKNLVVSRVTEDSARLSWTAPDAAFDSFFIGYLEPQPPGEAIILGVPGSERSYDLTGLKPGTEYSVLIHGVKGGLLSSPLHAYSTT

SEQ ID NO: 168 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGLLSSPLSAIFTT

SEQ ID NO: 169 - protein, 90 aa, synthetic construct
MLPAPKNLVVSRVTEDSARLSWTAPDAAFDSFYIAYAEPRPDGEAILLLVPGSERSYDLTGLKPGTEYSVLIHGVKGGLLSSPLSAVFTT

SEQ ID NO: 170 - protein, 90 aa, synthetic construct
MLPAPKNLVVSRVTEDSARLSWTAPDAAFDSFYIAYAEPRPDGEAIHLLVPGSERSYDLTGLKPGTEYSVLIHGVKGGLLSSPLLAHFTT

SEQ ID NO: 171 - protein, 90 aa, synthetic construct
MLPAPKNLVVSRVTEDSARLSWTAPDAAFDSFYIAYAEPRPDGEAIQLWVPGSERSYDLTGLKPGTEYSVLIHGVKGGLLSSPLDAYFTT

SEQ ID NO: 172 - protein, 89 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLASFTT

SEQ ID NO: 173 - protein, 90 aa, synthetic construct
MLPAPKNLVVSRVTEDSARLSWTAPDAAFDSFHIVYHEPRPSGEAIHLQVPGSERSYDLTGLKPGTEYNVTIQGVKGGFPSYPLSAFFTT

SEQ ID NO: 174 - protein, 90 aa, synthetic construct
MLPAPKNLVVSRVTEDSARLSWTAPDAAFDSFFIGYLEPQPPGEAIQLWVPGSERSYDLTGLKPGTEYNVTIQGVKGGFPSMPLSAIFTT

SEQ ID NO: 175 - protein, 90 aa, synthetic construct
MLPAPKNLVVSRVTEDSARLSWTAPDAAFDSFRISYCETFYHGEAIVLTVPGSERSYDLTGLKPGTEYSVLIHGVKGGLLSSPLIAKFTT

SEQ ID NO: 176 - protein, 90 aa, synthetic construct
MLPAPKNLVVSRVTEDSARLSWTAPDAAFDSFYIAYAEPRPDGEAIWLKVPGSERSYDLTGLKPGTEYSVLIHGVKGGLLSSPLQANFTT

SEQ ID NO: 177 - protein, 90 aa, synthetic construct; identical to SEQ ID NO: 176

SEQ ID NO: 178 - protein, 90 aa, synthetic construct
MLPAPKNLVVSRVTEDSARLSWTAPDAAFDSFYIAYAEPRPDGEAIQLQVPGSERSYDLTGLKPGTEYSVLIHGVKGGLLSSPLSAIVTT

SEQ ID NO: 179 - protein, 90 aa, synthetic construct
MLPAPKNLVVSRVTEDSARLSWTAPDAAFDSFPIAYAEPRPDGEAIVLTVPGSERSYDLTGLKPGTEYSVLIHGVKGGLLSSPLSAFFTT

SEQ ID NO: 180 - protein, 90 aa, synthetic construct
MLPAPKNLVVSRVTEDSARLSWTAPDAAFDSFYIAYAEPRPDGEAIALLVPGSERSYDLTGLKPGTEYSVLIHGVKGGLLSSPLVAQFTT

SEQ ID NO: 181 - protein, 90 aa, synthetic construct; identical to SEQ ID NO: 161

SEQ ID NO: 182 - protein, 90 aa, synthetic construct
MLPAPKNLVVSRVTEDSARLSWTAPDAAFDSFYIAYAEPRPDGEAIDLHVPGSERSYDLTGLKPGTEYSVLIHGVKGGLLSSPLHALFTT

SEQ ID NO: 183 - protein, 90 aa, synthetic construct
MLPAPKNLVVSRVTEDSARLSWTAPDAAFDSFYIAYAEPRPDGEAILLDVPGSERSYDLTGLKPGTEYSVLIHGVKGGFPSMPLSAIFTT

SEQ ID NO: 184 - protein, 88 aa, synthetic construct
MLPAPKNLVVSRVTEDSARLSWTAPDAAFDSFFIGYLEPQPPGEAIDLAVPGSERSYDLTGLKPGTEYSVLIHGVKGGLLSSPLSFTT

SEQ ID NO: 185 - protein, 90 aa, synthetic construct
MLPAPKNLVVSRVTEDSARLSWTAPDAAFDSFYIAYAEPRPDGEAIYLGVPGSERSYDLTGLKPGTEYSVLIHGVKGGLLSSPLRAKFTT

SEQ ID NO: 186 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLSAIFTT

SEQ ID NO: 187 - protein, 88 aa, synthetic construct
MLPAPKNLVVSRVTEDSARLSWTAPDAAFDSFFIGYLEPQPPGEAISLLVPDSERSYDLTGLKPGTEYNVTIQGVKGGFPSMPLKFTT

SEQ ID NO: 188 - protein, 90 aa, synthetic construct
MLPAPKNLVVSRVTEDSARLSWTAPDAAFDSFYIAYAEPRPDGEAIGLGVPGSERSYDLTGLKPGTEYSVLIHGVKGGLLSSPLDASFTT

SEQ ID NO: 189 - protein, 90 aa, synthetic construct
MLPAPKNLVVSRVTEDSARLSWTAPDAAFDSFYIAYAEPRPDGEAIQLTVPGSERSYDLTGPKPGTEYWVLIQGVKGGGSSVPLVAYFTT

SEQ ID NO: 190 - protein, 90 aa, synthetic construct
MLPAPKNLVVSRVTEDSARLSWTAPDAAFDSFYIAYAEPRPDGEAISLDVPGSERSYDLTGLKPGTEYSVLIHGVKGGLLSSPLEASFTT

SEQ ID NO: 191 - protein, 90 aa, synthetic construct
MLPAPKNLVVSRVTEDSARLSWTAPDAAFDSFFIGYLEPQPPGEAIILAVPGSERSYDLTGLKPGTEYNVTIQGVKGGFPSLPLVASFTT

SEQ ID NO: 192 - protein, 90 aa, synthetic construct
MLPAPKNLVVSRVTEDSARLSWTAPDAAFDSFDIGYTEYGGYGEAIYLSVPGSERSYDLTGLKPGTEYWVLIQGVKGGGSSVPLSAIFTT

SEQ ID NO: 193 - protein, 90 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPLIANFTT

SEQ ID NO: 194 - protein, 90 aa, synthetic construct
MLPAPKNLVVSRVTEDSARLSWTAPDAAFDSFYIAYAEPRPDGEAIALLVPGSERSYDLTGLKPGTEYSVLIHGVKGGLLSSPLSAIVTT

SEQ ID NO: 195 - protein, 90 aa, synthetic construct
MLPAPKNLVVSRVTEDSARLSWTAPDAAFDSFYIAYAEPRPDGEAIILDVPGSERSYDLTGLKPGTEYSVLIHGVKGGLLSSPLSSIFTT

SEQ ID NO: 196 - protein, 90 aa, synthetic construct
MLPAPKNLVVSRVTEDSARLSWTAPDAAFDSFYIAYAEPRPDGEAIVLWVPGSERSYDLTGLKPGTEYSVLIHGVKGGLLSSPLRASFTT

SEQ ID NO: 197 - protein, 90 aa, synthetic construct
MLPAPKNLVVSRVTEDSARLSWTAPDAAFDSFYIAYAEPRPDGEAIKLDVPGSERSYDLTGLKPGTEYSVLIHGVKGGLLSSPLVAFFTT

SEQ ID NO: 198 - protein, 90 aa, synthetic construct
MLPAPKNLVVSRVTEDSARLSWTAPDAAFDSFYIAYAEPRPDGEAIILEVPGSERSYDLTGLKPGTEYSVLIHGVKGGLLSSPLVAYFTT

SEQ ID NO: 199 - protein, 90 aa, synthetic construct
MLPAPKNLVVSRVTEDSARLSWTAPDAAFDSFYIAYAEPRPDGEAIHLWVPGSERSYDLTGLKPGTEYSVLIHGVKGGLLSSPLHADFTT

SEQ ID NO: 200 - protein, 90 aa, synthetic construct
MLPAPKNLVVSRVTEDSARLSWTAPDAAFDSFYIAYAEPRPDGEAIWLEVPGSERSYDLTGLKPGTEYSVLIHGVKGGLLSSPLVADFTT

SEQ ID NO: 201 - protein, 90 aa, synthetic construct
MLPAPKNLVVSRVTEDSARLSWTAPDAAFDSFYIAYAEPRPDGEAISLWVPGSERSYDLTGLKPGTEYSVLIHGVKGGLLSSPLLAHFTT

SEQ ID NO: 202 - protein, 89 aa, synthetic construct
MLPAPKNLVVSRVTEDSARLSWTAPDAAFDSFDIGYTEYGGYGEAILHVPGSERSYDLTGLKPGTEYWVLIQGVKGGGSSVPLSAIFTT

SEQ ID NO: 203 - protein, 90 aa, synthetic construct
MLPAPKNLVVSRVTEDSARLSWTAPDAAFDSFFIGYLEPQPPGEAIVLLVPGSERSYDLTGLKPGTEYNVTIQGVKGGFPSVPLAAFFTT

SEQ ID NO: 204 - protein, 85 aa, synthetic construct
MLPAPKNLVVSRVTEDSARLSWTAPDAAFDSFFIGYLEPQPPGEAILLWVPGSERSYDLTGLKPGTEYNVTIQGVKGGFPSQFTT

SEQ ID NO: 205 - protein, 93 aa, synthetic construct
MLPAPKNLVVSRVTEDSARLSWTAPDAAFDSFFIGYLEPQPPGEAILLGVPGSERSYDLTGLKPGTEYNVTIQGVKGGFPSMPLHPLVALFTT

SEQ ID NO: 206 - protein, 90 aa, synthetic construct
MLPAPKNLVVSRVTEDSARLSWTAPDAAFDSFYIAYAEPRPDGEAIGLDVPGSERSYDLTGLKPGTEYSVLIHGVKGGLLSSPLSAIFTT

SEQ ID NO: 207 - protein, 90 aa, synthetic construct
MLPAPKNLVVSRVTEDSARLSWTAPDAAFDSFYIAYAEPRPDGEAIHLSVPGSERSYDLTGLKPGTEYSVLIHGVKGGLLSSPLAAYFTT

SEQ ID NO: 208 - protein, 90 aa, synthetic construct
MLPAPKNLVVSRVTEDSARLSWTAPDAAFDSFFIGYLEPQPPGEAIVLAVPGSERSYDLTGLKPGTEYNVTIQGVKGGFPSYPLVAAFTT

SEQ ID NO: 209 - protein, 90 aa, synthetic construct
MLPAPKNLVVSRVTEDSARLSWTAPDAAFDSFYIAYAEPRPDGEAILLQVPGSCRSYDLTGLKPGTEYSVLIHGVKGGLLSSPLTAIFTT

SEQ ID NO: 210 - protein, 90 aa, synthetic construct
MLPAPKNLVVSRVTEDSARLSWTAPDAAFDSFFIGYLEPQPPGEAINLQVPGSERSYDLTGLKPGTEYNVTIQGVKGGFPSFPLSAVFTT

SEQ ID NO: 211 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLVAIFTT

SEQ ID NO: 212 - protein, 90 aa, synthetic construct
MLPAPKNLVVSRVTEDSARLSWTAPDAAFDSFYIAYAEPRPDGEAIWLAVPGSERSYDLTGLKPGTEYSVLIHGVKGGLLSSPLHAQFTT

SEQ ID NO: 213 - protein, 87 aa, synthetic construct
MLPAPKNLVVSRVTEDSARLSWTAPDAAFDSFYIAYAEPRPDGEAILLGVPGSERSYDLTGLKPGTEYSVLIHGVKGGLLSSPLFTT

SEQ ID NO: 214 - protein, 90 aa, synthetic construct
MLPAPKNLVVSRVTEDSARLSWTAPDAAFDSFYIAYAEPRPDGEAIGLQVPGSERSYDLTGLKPGTEYSVLIHGVKGGLLSSPLCAEFTT

SEQ ID NO: 215 - protein, 90 aa, synthetic construct
MLPAPKNLVVSRVTEDSARLSWTAPDAAFDSFYIAYAEPRPDGEAIVLWVPGSERSYDLTGLKPGTEYSVLIHGVKGGLLSSPLIAEFTT

SEQ ID NO: 216 - protein, 88 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPPKFTT

SEQ ID NO: 217 - protein, 90 aa, synthetic construct
MLPAPKNLVVSRVTEDSARLSWTAPDAAFDSFYIAYAEPRPDGEAIILEVPGSERSYDLTGLKPGTEYSVLIHGVKGGLLSSPLRAVFTT

SEQ ID NO: 218 - protein, 90 aa, synthetic construct
MLPAPKNLVVSRVTEDSARLSWTAPDAAFDSFYIAYAEPRPDGEAIHLVVPGSERSYDLTGLKPGTEYSVLIHGVKGGLLSSPLSAIFTT

SEQ ID NO: 219 - protein, 90 aa, synthetic construct
MLPAPKNLVVSRVTEDSARLSWTAPDAAFDSFYIAYAEPRPDGEAISLKVPGSERSYDLTGLKPGTEYSVLIHGVKGGLLSSPLEAIFTT

SEQ ID NO: 220 - protein, 114 aa, synthetic construct
MLPAPKNPVVSRVTEDSARLSWTAPDAAFDSFYIAYAEPRPDGEAIHLLVPGSERSYDLTGLKPGTEYSVLIHGVKGGLLSSPLKRLSPPVVTITITMAVCRKPVAENLSQTLS

SEQ ID NO: 221 - protein, 89 aa, synthetic construct
MLPAPKNLVVSRVTEDSARLSWTAPDAAFDSFFIGYLEPQPPGEAIFLDVPGSERSYDLTGLKPGTEYNVTIQGVKGGFPSPLTAFFTT

SEQ ID NO: 222 - protein, 90 aa, synthetic construct
MLPAPKNLVVSRVTEDSARLSWTAPDAAFDSFFIGYLEPQPPGEAIVLDVPGSERSYDLTGLKPGTEYNVTIQGVKGGFPSHPLAAAFTT

SEQ ID NO: 223 - protein, 90 aa, synthetic construct
MLPAPKNLVVSRVTEDSARLSWTAPDAAFDSFFIGYLEPQPPGEAIGLAVPGSERSYDLTGLKPGTEYNVTIQGVKGGFPSVPLQANFTT

SEQ ID NO: 224 - protein, 90 aa, synthetic construct
MLPAPKNLVVSRVTEDSARLSWTAPDAAFDSFYIAYAEPRPDGEAILLRVPGSERSYDLTGLKPGTEYSVLIHGVKGGLLSSPLVAEFTT

SEQ ID NO: 225 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLSASFTT

SEQ ID NO: 226 - protein, 90 aa, synthetic construct
MLPAPKNLVVSRVTEDSARLSWTAPDAAFDSFYIAYAEPRPDGEAIGLHVPGSERSYDLTGLKPGTEYSVLIHGVKGGLLSSPLTASFTT

SEQ ID NO: 227 - protein, 90 aa, synthetic construct
MLPAPKNLVVSRVTEDSARLSWTAPDAAFDSFYIAYAEPRPDGEAIGLRVPGSERSYDLTGLKPGTEYSVLIHGVKGGLLSSPLSAIFTT

SEQ ID NO: 228 - protein, 90 aa, synthetic construct
MLPAPKNLVVSRVTEDSARLSWTAPDAAFDSFYIAYAEPRPDGEAIVLRVPGSERSYDLTGLKPGTEYSVLIHGVKGGLLSSPLAASFTT

SEQ ID NO: 229 - protein, 90 aa, synthetic construct
MLPAPKNLVVSRVTEDSARLSWTAPDAAFDSFYIAYAEPRPDGEAIQLLVPGSERSYDLTGLKPGTEYSVLIHGVKGGLLSSPLVAHFTT

SEQ ID NO: 230 - protein, 90 aa, synthetic construct
MLPAPKNLVVSRVTEDSARLSWTAPDAAFDSFYIAYAEPRPDGEAIWLLVPGSERSYDLTGLKPGTEYSVLIHGVKGGLLSSPLVAFFTT

SEQ ID NO: 231 - protein, 90 aa, synthetic construct
MLPAPKNLVVSRVTEDSARLSWTAPDAAFDSFFIGYLEPQPPGEAIHLYVPGSERSYDLTGLKPGTEYNVTIQGVKGGFPSDPLDAVFTT

SEQ ID NO: 232 - protein, 85 aa, synthetic construct
MLPAPKNLVVSRVTEDSARLSWTAPDAAFDSFFIGYLEPQPPGEAIYLDVPGSERSYDLTGLKPGTEYNVTIQGVKGGFPSTFTT

SEQ ID NO: 233 - protein, 90 aa, synthetic construct
MLPAPKNLVVSRVTEDSARLSWTAPDAAFDSFYIAYAEPRPDGEAIQLFVPGSERSYDLTGLKPGTEYSVLIHGVKGGLLSSPLKAYFTT

SEQ ID NO: 234 - protein, 90 aa, synthetic construct
MLPAPKNLVVSRVTEDSARLSWTAPDAAFDSFYIAYAEPRPDGEAIVLVVPGSERSYDLTGLKPGTEYSVLIHGVKGGLLSSPLSAIFTT

SEQ ID NO: 235 - protein, 90 aa, synthetic construct
MLPAPKNLVVSRVTEDSARLSWTAPDAAFDSFFIGYLEPQPPGEAIQLTVPGSERSYDLTGLKPGTEYNVTIQGVKGGFPSLPLSADFTT

SEQ ID NO: 236 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLDAEFTT

SEQ ID NO: 237 - protein, 90 aa, synthetic construct
MLPAPKNLVVSRVTEDSARLSWTAPDAAFDSFYIAYAEPRPDGEAIVLAVPGSERSYDLTGLKPGTEYSVLIHGVKGGLLSSPLYASFTT

SEQ ID NO: 238 - protein, 88 aa, synthetic construct
MLPAPKNLVVSRVTEDSARLSWTTPDAAFDSFYIAYAEPRPDGEAIRLQVPGSERSYDLTGLKPGTEYSVLIHGVKGGLLSSPLGFTT

SEQ ID NO: 239 - protein, 90 aa, synthetic construct
MLPAPKNLVVSRVTEDSARLSWTAPDAAFDSFYIAYAEPRPDGEAIQLVVPGSERSYDLTGLKPGTEYSVLIHGVKGGLLSSPLYAIFTT

SEQ ID NO: 240 - protein, 90 aa, synthetic construct
MLPAPKNLVVSRVTEDSARLSWTAPDAAFDSFYIAYAEPRPDGEAISLSVPGSERSYDLTGLKPGTEYSVLIHGVKGGLLSSPLHAKFTT

SEQ ID NO: 241 - protein, 90 aa, synthetic construct
MLPAPKNLVVSRVTEDSARLSWTAPDAAFDSFFIGYLEPQPPGEAIHLGVPGSERSYDLTGLKPGTEYNVTIQGVKGGFPSIPLFASFTT

SEQ ID NO: 242 - protein, 90 aa, synthetic construct
MLPAPKNLVVSRVTEDSARLSWTAPDAAFDSFYIAYAEPRPDGEAIQLLVPGSERSYDLTGLKPGTEYSVLIHGVKGGLLSSPLYAAFTT

SEQ ID NO: 243 - protein, 90 aa, synthetic construct
MLPAPKNLVVSRVTEDSARLSWTAPDAAFDSFFIGYLEPQPPGEAIHLAVPGSERSYDLTGLKPGTEYNVTIQGVKGGFPSVPLAAVFTT

SEQ ID NO: 244 - protein, 90 aa, synthetic construct
MLPAPKNLVVSRVTEDSARLSWTAPDAAFDSFYIAYAEPRPDGEAISLQVPGSERSYDLTGLKPGTEYSVLIHGVKGGLLSSPLGAHFTT

SEQ ID NO: 245 - protein, 90 aa, synthetic construct
MLPAPKNLVVSRVTEDSARLSWTAPDAAFDSFYIAYAEPRPDGEAIALWVPGSERSYDLTGLKPGTEYSVLIHGVKGGLLSSPLVASFTT

SEQ ID NO: 246 - protein, 90 aa, synthetic construct
MLPAPKNLVVSRVTEDSARLSWTAPDAAFDSFYIAYAEPRPDGEAIQLHVPGSERSYDLTGLKPGTEYSVLIHGVKGGLLSSPLYAFFTT

SEQ ID NO: 247 - protein, 90 aa, synthetic construct
MLPAPKNLVVSRVTEDSARLSWTAPDAAFDSFYIAYAEPRPDGEAILLHVPGSERSYDLTGLKPGTEYSVLIHGVKGGLLSSPLRASFTT

SEQ ID NO: 248 - protein, 90 aa, synthetic construct
MLPAPKNLVVSRVTEDSARLSWTAPDAAFDSFYIAYAEPRPDGEAIWLGVPGSERSYDLTGLKPGTEYSVLIHGVKGGLLSSPLHATFTT

SEQ ID NO: 249 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLHANFTT

SEQ ID NO: 250 - protein, 90 aa, synthetic construct
MLPAPKNLVVSRVTEDSARLSWTAPDAAFDSFYIAYAEPRPDGEAILLRVPGSERSYDLTGLKPGTEYSVLIHGVKGGLLSSPLYAKFTT

SEQ ID NO: 251 - protein, 90 aa, synthetic construct
MLPAPKNLVVSRVTEDSARLSWTAPDAAFDSFFIGYLEPQPPGEAIGLWVPGSERSYDLTGLKPGTEYNVTIQGVKGGFPSDPLQAVFTT

SEQ ID NO: 252 - protein, 90 aa, synthetic construct
MLPAPKNLVVSRVTEDSARLSWTAPDAAFDSFYIAYAEPRPDGEAIVLHVPGSERSYDLTGLKPGTEYSVLIHGVKGGLLSSPLDAFFTT

SEQ ID NO: 253 - protein, 90 aa, synthetic construct
MLPAPKNLVVSRVTEDSARLSWTAPDAAFDSFYIAYAEPRPDGEAIILHVPGSERSYDLTGLKPGTEYSVLIHGVKGGLLSSPLDAYFTT

SEQ ID NO: 254 - protein, 90 aa, synthetic construct
MLPAPKNLVVSRVTEDSARLSWTAPDAAFDSFYIAYAEPRPDGEAILLAVPGSERSYDLTGLKPGTEYSVLIHGVKGGLLSSPLSAKFTT

SEQ ID NO: 255 - protein, 90 aa, synthetic construct
MLPAPKNLVVSRVTEDSARLSWTAPDAAFDSFFIGYLEPQPPGEAILLFVPGSERSYDLTGLKPGTEYNVTIQGVKGGFPSTPLSASFTT

SEQ ID NO: 256 - protein, 90 aa, synthetic construct
MLPAPKNLVVSRVTEDSARLSWTAPDAAFDSFYIAYAEPRPDGEAIQLTVPGSERSYDLTGLKPGTEYSVLIHGVKGGLLSSPLHAYFTT

SEQ ID NO: 257 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLRAYFTT

SEQ ID NO: 258 - protein, 90 aa, synthetic construct
MLPAPKNLVVSRVTEDSARLSWTAPDAAFDSFYIAYAEPRPDGEAILLEVPGSERSYDLTGLKPGTEYSVLIHGVKGGLLSSPLVAFFTT

SEQ ID NO: 259 - protein, 90 aa, synthetic construct
MLPAPKNLVVSRVTEDSARLSWTAPDAAFDSFYIAYAEPRPDGEAIQLGVPGSERSYDLTGLKPGTEYSVLIHGVKGGLLSSPLLAVFTT

SEQ ID NO: 260 - protein, 90 aa, synthetic construct
MLPAPKNLVVSRVTEDSARLSWTAPDAAFDSFYIAYAEPRPDGEAIHLRVPGSERSYDLTGLKPGTEYNVTIQGVKGGFPSMPLSAIFTT

SEQ ID NO: 261 - protein, 90 aa, synthetic construct
MLPAPKNLVVSRVTEDSARLSWTAPDAAFDSFYIAYAEPRPDGEAILLQVPGSERSYDLTGLKPGTEYSVLIHGVKGGLLSSPLIAKFTT

SEQ ID NO: 262 - protein, 90 aa, synthetic construct
MLPAPKNLVVSRVTEDSARLSWTAPDAAFDSFYIAYAEPRPDGEAIVLHVPGSERSYDLTGLKPGTEYSVLIHGVKGGLLSSPLQAIFTT

SEQ ID NO: 263 - protein, 90 aa, synthetic construct
MLPAPKNLVVSRVTEDSARLSWTAPDAAFDSFYIAYAEPRPDGEAIALVVPGSERSYDLTGLKPGTEYSVLIHGVKGGLLSSPLAANFTT

SEQ ID NO: 264 - protein, 90 aa, synthetic construct
MLPAPKNLVVSRVTEDSARLSWTAPDAAFDSFYIAYAEPRPDGEAINLSVPGSERSYDLTGLKPGTEYSVLIHGVKGGLLSSPLDAYFTT

SEQ ID NO: 265 - protein, 90 aa, synthetic construct
MLPAPKNLVVSRVTEDSARLSWTAPDAAFDSFYIAYAEPRPDGEAIVLEVPGSERSYDLTGLKPGTEYSVLIHGVKGGLLSSPLTASFTT

SEQ ID NO: 266 - protein, 90 aa, synthetic construct
MLPAPKNLVVSRVTEDSARLSWTAPDAAFDSFYIAYAEPRPDGEAIRLQVPGSERSYDLTGLKPGTEYSVLIHGVKGGLLSSPLGASFTT

SEQ ID NO: 267 - protein, 90 aa, synthetic construct
MLPAPKNLVVSRVTEDSARLSWTAPDAAFDSFYIAYAEPRPDGEAIGLWVPGSERSYDLTGLKPGTEYSVLIHGVKGGLLSSPLVAYFTT

SEQ ID NO: 268 - protein, 87 aa, synthetic construct
MLPAPKNLVVSRVTEDSARLSWTAPDAAFDSFYIAYAEPRPDGEAIYLEVPGSERSYDLTGLKPGTEYSVLIHGVKGGLLSSPLFTT

SEQ ID NO: 269 - protein, 90 aa, synthetic construct
MLPAPKNLVVSRVTEDSARLSWTAPDAAFDSFYIAYAEPRPDGEAIWLDVPGSERSYDLTGLKPGTEYSVLIHGVKGGLLSSPLDAYFTT

SEQ ID NO: 270 - protein, 90 aa, synthetic construct
MLPAPKNLVVSRVTEDSARLSWTAPDAAFDSFAIEYCETKMCGEAIVLTVPGSERSYDLTGLKPGTEYRVPIPGVKGGTASLPLSAIFTT

SEQ ID NO: 271 - protein, 90 aa, synthetic construct
MLPAPKNLVVSRVTEDSARLSWTAPDAAFDSFFIYYIESYPAGEAIVLTVPGSERSYDLTGLKPGTEYWVGIDGVKGGRWSTPLSAIFTT

SEQ ID NO: 272 - protein, 90 aa, synthetic construct
MLPAPKNLVVSRVTEDSARLSWTAPDAAFDSFFIYYIESYPAGEAIVLTVPGSCRSYDLTGLKPGTEYWVGIDGVKGGRWSTPLSAIFTT

SEQ ID NO: 273 - protein, 90 aa, synthetic construct
  VARIANT 33: note = D, F, Y, or H
  VARIANT 35: note = Y, G, A, or V
  VARIANT 37: note = I, T, L, A, or H
  VARIANT 39: note = S, Y or P
  VARIANT 40: note = Y, G, Q, or R
  VARIANT 41: note = G or P
  VARIANT 42: note = A, Y, P, D, or S
  VARIANT 47: note = A, C, D, E, F, G, H, I, K, L, N, Q, R, S, T, V, W, or Y
  VARIANT 49: note = A, C, D, E, F, G, H, I, K, L, N, Q, R, S, T, V, W, or Y
  VARIANT 69: note = W, N, S, or E
  VARIANT 71: note = L, Y, or G
  VARIANT 73: note = D, Q, H, or V
  VARIANT 74: note = G or S
  VARIANT 79: note = R, G, F, L, or D
  VARIANT 80: note = W, S, P, or L
  VARIANT 82: note = T, V, M, or S
  VARIANT 85: note = A, C, D, E, F, G, H, I, K, L, N, Q, R, S, T, V, W, or Y
  VARIANT 87: note = A, C, D, E, F, G, H, I, K, L, N, Q, R, S, T, V, W, or Y
MLPAPKNLVVSRVTEDSARLSWTAPDAAFDSFXIXYXEXXXXGEAIXLXVPGSERSYDLTGLKPGTEYXVXIXXVKGGXXSXPLXAXFTT

SEQ ID NO: 274 - protein, 291 aa, Homo sapiens
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 275 - protein, 230 aa, Homo sapiens
QNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 276 - protein, 89 aa, synthetic construct
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 277 - protein, 89 aa, synthetic construct
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 278 - protein, 4 aa, synthetic construct
GSGS

SEQ ID NO: 279 - protein, 8 aa, synthetic construct
GGGSGGGS

SEQ ID NO: 280 - protein, 25 aa, synthetic construct
  VARIANT 1..25: note = This sequence may encompass 1-5 "Gly Gly Gly Gly Ser" repeating units
GGGGSGGGGSGGGGSGGGGSGGGGS

SEQ ID NO: 281 - protein, 4 aa, synthetic construct
APAP

SEQ ID NO: 282 - protein, 10 aa, synthetic construct
APAPAPAPAP

SEQ ID NO: 283 - protein, 20 aa, synthetic construct
APAPAPAPAPAPAPAPAPAP

SEQ ID NO: 284 - protein, 40 aa, synthetic construct
APAPAPAPAPAPAPAPAPAPAPAPAPAPAPAPAPAPAPAP

SEQ ID NO: 285 - protein, 29 aa, synthetic construct
AEAAAKEAAAKEAAAKEAAAKEAAAKAAA

SEQ ID NO: 286 - RNA, 19 nt, synthetic construct
  source 1..19: mol_type = other RNA
tctcgtggccttaatgaaa

SEQ ID NO: 287 - RNA, 20 nt, synthetic construct
  source 1..20: mol_type = other RNA
ttcattaaggccacgagatt

SEQ ID NO: 288 - protein, 90 aa, synthetic construct
MLPAPKNLVVSRVTEDSARLSWTAPDAAFDSFYIAYAEPRPDGEAIVLTVPGSERSYDLTGLKPGTEYSVLIHGVKGGLLSSPLSAIFTT

SEQ ID NO: 289 - protein, 90 aa, synthetic construct
MLPAPKNLVVSRVTEDSARLSWTAPDAAFDSFFIGYLEPQPPGEAIVLTVPGSERSYDLTGLKPGTEYNVTIQGVKGGFPSMPLSAIFTT

SEQ ID NO: 290 - protein, 90 aa, synthetic construct
MLPAPKNLVVSRVTEDSARLSWTAPDAAFDSFHIVYHEPRPSGEAIVLTVPGSERSYDLTGLKPGTEYEVGIVSVKGGDLSVPLSAIFTT

SEQ ID NO: 291 - protein, 90 aa, synthetic construct
MLPAPKNLVVSRVTEDSARLSWTAPDAAFDSFDIGYTEYGGYGEAIVLTVPGSERSYDLTGLKPGTEYWVLIQGVKGGGSSVPLSAIFTT

SEQ ID NO: 292 - protein, 189 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTAPAPAPAPAPLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 293 - protein, 199 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTGGGGSGGGGSGGGGSGGGGSLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 294 - protein, 199 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTEAAAKEAAAKEAAAKEAAAKLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 295 - protein, 184 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTTEAAAKLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 296 - protein, 189 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTAPAPAPAPAPLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 297 - protein, 199 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTGGGGSGGGGSGGGGSGGGGSLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 298 - protein, 199 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTEAAAKEAAAKEAAAKEAAAKLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 299 - protein, 184 aa, synthetic construct
MLPAPKNLVVSRVTEDSARLSWTAPDAAFDSFKIEYFEYVGYGEAIVLTVPGSCRSYDLTGLKPGTEYYVAIYGVKGGWYSRPLSAIFTTEAAAKLPAPKNLVVSRVTEDSARLSWTAPDAAFDSFKIEYFEYVGYGEAIVLTVPGSERSYDLTGLKPGTEYYVAIYGVKGGWYSRPLSAIFTT

SEQ ID NO: 300 - protein, 20 aa, synthetic construct
EAAAKEAAAKEAAAKEAAAK

SEQ ID NO: 301 - protein, 20 aa, synthetic construct
GGGGSGGGGSGGGGSGGGGS

SEQ ID NO: 302 - protein, 10 aa, synthetic construct
APAPAPAPAP

SEQ ID NO: 303 - protein, 5 aa, synthetic construct
EAAAK

SEQ ID NO: 304 - protein, 90 aa, synthetic construct
MLPAPKNLVVSRVTEDSARLSWTAPDAAFDSFAIVYHEPRPDGEAIVLTVPGSCRSYDLTGLKPGTEYEVVILGVKGGVHSYPLSAIFTT

SEQ ID NO: 305 - protein, 90 aa, synthetic construct
MLPAPKNLVVSRVTEDSARLSWTAPDAAFDSFAIVYHEPRPDGEAIVLTVPGSERSYDLTGLKPGTEYEVVILGVKGGVHSYPLSAIFTT

SEQ ID NO: 306 - protein, 90 aa, synthetic construct
MLPAPKNLVVSRVTEDSARLSWTAPDAAFDSFFIGYLEPQPPGEAIHLGVPGSCRSYDLTGLKPGTEYNVTIQGVKGGFPSIPLFASFTT

SEQ ID NO: 307 - protein, 25 aa, synthetic construct
  VARIANT 1..25: note = This sequence may encompass 1-5 "Glu Ala Ala Ala Lys" repeating units
EAAAKEAAAKEAAAKEAAAKEAAAK

SEQ ID NO: 308 - protein, 5 aa, synthetic construct
GGGGS

SEQ ID NO: 309 - protein, 6 aa, synthetic construct
HHHHHH

SEQ ID NO: 310 - skipped

SEQ ID NO: 311 - protein, 40 aa, synthetic construct; identical to SEQ ID NO: 284
  VARIANT 1..40: note = This sequence may encompass 1-20 "Ala Pro" repeating units